(19) United States
(12) Patent Application Publication
(10) Pub. No.: US 2026/0071222 A1
(43) Pub. Date: Mar. 12, 2026

(54) SPLICE-SWITCHING OLIGONUCLEOTIDES TARGETING IL-4RA

(71) Applicants: AGENCY FOR SCIENCE, TECHNOLOGY AND RESEARCH (A*STAR), Singapore (SG); NATIONAL SKIN CENTRE (SINGAPORE) PTE LTD, Singapore (SG)

(72) Inventors: Dwi Sarengat PRAMONO, Singapore (SG); Keng Boon Dave WEE, Singapore (SG); Hong Liang TEY, Singapore (SG)

(21) Appl. No.: 19/106,447

(22) PCT Filed: Aug. 22, 2023
(86) PCT No.: PCT/SG2023/050576

§ 371 Date: Feb. 25, 2025

(30) Foreign Application Priority Data
Aug. 25, 2022 (SG) 10202250819E

(51) Int. Cl. C12N 15/113; A61K 31/712; A61K 31/7125; A61P 29/00

(52) U.S. Cl. CPC C12N 15/1138; A61K 31/712; A61K 31/7125; A61P 29/00; C12N 2310/11; C12N 2310/315; C12N 2310/321; C12N 2320/33

(57) ABSTRACT

The present invention relates generally to the field of RNA splicing. In particular, the invention relates to splice-switching oligonucleotides (SSOs) configured to alter the splicing of a IL-4Rα pre-mRNA. The invention also relates to a method of exon-skipping wherein the binding of the SSO to a IL-4Rα pre-mRNA induces the exclusion of an exon during splicing of the IL-4Rα pre-mRNA to a IL-4Rα mature mRNA. The invention also relates to the use of SSOs as therapeutic candidates for treating Th2-mediated inflammatory diseases.

### FIELD OF THE INVENTION

The present invention relates generally to the field of RNA splicing. In particular, the invention relates to splice-switching oligonucleotides (SSOs) configured to alter the splicing of a IL-4Rα pre-mRNA. The invention also relates to the use of SSOs as therapeutic candidates for atopic dermatitis.

### BACKGROUND OF THE INVENTION

Atopic dermatitis (AD) is a chronic debilitating disease which negatively impacts many aspects of the lives of patients and their family members. The disease affects 10-20% of Singapore's population. It is characterized by dry skin, itch and inflammation. Skin moisturizing is the first line management in AD. While simple moisturizing helps with the abovementioned symptoms, it is not anti-inflammatory and patients have to resort to immunosuppressants which cause prominent side effects.

Recently, a biologic drug, Dupilumab has been approved for treating AD in Singapore and it has shown good clinical efficacy in patients suffering from severe AD. Dupilumab is a human monoclonal antibody designed to target IL-4Rα and disrupts IL-4 and IL-13 signalling that drive the persistent inflammation in AD. With tolerable side effects such as conjunctivitis and herpes virus reactivation, Dupilumab has given significant relief to many patients who have suffered the debilitating disease for many years. However, Dupilumab is required to be administered systemically by injection, does not cater to the vast majority of AD patients (75%) who have mild to moderate disease, and is currently not suitable for children under 6 years old. Given that AD is a chronic disease, long-term systemic administration of anti-IL-4Rα may result in long-term memory loss and dementia, and Kawasaki disease.

There is therefore a need to develop a safe, local and anti-inflammatory agent to prevent escalation to the severe condition. Furthermore, other desirable features and characteristics will become apparent from the subsequent detailed description and the appended claims, taken in conjunction with the accompanying drawings and this background of the disclosure.

### SUMMARY

In one aspect, there is provided a splice-switching oligonucleotide (SSO) that binds to a IL-4Ra pre-mRNA, wherein the SSO comprises a sequence selected from the group consisting of SEQ ID NOs 1 to 8, and wherein binding of the SSO induces the exclusion of an exon during splicing of the IL-4Rα pre-mRNA to a IL-4Rα mature mRNA.

In one embodiment, at least one of the nucleotides of the SSO as described herein is chemically modified and wherein the chemical modification is 2′-O-methyl RNA modification, 2′-O-methoxyethyl RNA modification or phosphorothioate linkage.

In one embodiment, each nucleotide of the SSO as described herein comprises either a 2′-O-methyl RNA modification or a 2′-O-methoxyethyl RNA modification.

In one embodiment, the SSO as described herein comprises phosphorothioate linkages between all nucleotides of the SSO.

In another aspect, there is provided an SSO as described herein for use as a medicament or in therapy.

In one embodiment, there is provided the SSO as described herein for use in the treatment of a Th2-mediated inflammatory disease.

In one embodiment, the Th2-mediated inflammatory disease is selected from the group consisting of atopic dermatitis, asthma, allergic rhinitis, allergic conjunctivitis and ulcerative colitis.

In another aspect, there is provided a use of an SSO as described herein in the manufacture of a medicament for treating a Th2-mediated inflammatory disease.

In one embodiment, the Th2-mediated inflammatory disease is selected from the group consisting of atopic dermatitis, asthma, allergic rhinitis, allergic conjunctivitis and ulcerative colitis.

In one aspect, there is provided a method of treating a Th2-mediated inflammatory disease comprising administering to a subject a composition comprising an SSO as described herein.

In one embodiment, the Th2-mediated inflammatory disease is selected from the group consisting of atopic dermatitis, asthma, allergic rhinitis, allergic conjunctivitis, and ulcerative colitis.

In another aspect, there is provided a pharmaceutical composition comprising (a) a therapeutically effective amount of an SSO as described herein and (b) one or more pharmaceutically acceptable carriers and/or diluents.

In one aspect, there is provided a method of exon-skipping comprising providing an SSO having a sequence selected from the group consisting of SEQ ID NOs 1 to 8, wherein the binding of the SSO to a IL-4Rα pre-mRNA induces the exclusion of an exon during splicing of the IL-4Rα pre-mRNA to a IL-4Rα mature mRNA.

In one embodiment, the exclusion of the exon causes a reduction in the levels of the IL-4Rα mature mRNA or functional protein.

In one embodiment, the method as described herein comprises providing an SSO with at least one of the nucleotides of the SSO being chemically modified and wherein the chemical modification is 2′-O-methyl RNA modification, 2′-O-methoxyethyl RNA modification or phosphorothioate linkage.

In one embodiment, the method as described herein comprises providing an SSO in which each nucleotide of the SSO comprises either a 2′-O-methyl RNA modification or a 2′-O-methoxyethyl RNA modification.

In one embodiment, the method as described herein comprises providing an SSO comprising phosphorothioate linkages between all nucleotides of the SSO.

### BRIEF DESCRIPTION OF THE DRAWINGS

The invention will be better understood with reference to the detailed description when considered in conjunction with the non-limiting examples and the accompanying drawings, in which:

FIG. 1 and Table 1 show the annotated isoforms of IL-4Rα gene according to Ensembl and NCBI. The size of exon, in bp, indicated at the bottom.

FIG. 2 shows the expression of IL-4Rα isoforms in HaCaT cells by Genescan. Genescan PCRs were performed using primer sets IL4R-3F (forward primer sitting on exon 3) and IL4R-8R (reverse primer sitting on exon 8) to analyse for relative abundance of exons 4, 5, 6 and 7 in untreated HaCat cells and HaCat cells treated with scrambled SSO without the presence of CHX (FIG. 2A) and with CHX (FIG. 2B). FIG. 2C shows Genescan PCRs were performed using primer sets IL4R-6F (forward primer sitting on exon 6) and IL4R-10/11R (reverse primer sitting on exons 10 and 11 junction) to analyse for relative abundance of exons 7, 8 and 9 in untreated HaCat cells and HaCat cells treated with scrambled SSO in the absence of CHX.

FIG. 3 shows SSO efficacy in inducing target exon skipping by gel electrophoresis after PCR. The SSOs and target exons are listed in Table 3 below. Each SSO was transfected at 50 nM in the presence of CHX on HaCaT cells. The image is a gel electrophoresis of Reverse Transcriptase PCR products of specific IL-4Rα transcript regions amplified from total RNA isolated from cells treated with indicated SSO. PCRs were performed with primers encompassing the exon targeted by the SSO, with 50 ng cDNA. Dark and light arrows indicate the amplicons of native transcripts without exon skipping/alteration of IL-4Rα and GAPDH, respectively. PCR of IL-4Rα transcripts from cells treated with SSOs 1872 and 1871 targeting exon 2 and SSO 1789 targeting exon 3 used primers IL4R-1F and IL4R-5R. Those treated with SSOs 1571 and 1790 targeting exon 4 and SSO 1572 targeting exon 5 used primers IL4R-3F and IL4R-7R. Those treated with SSO 1791 targeting exon 6 and SSOs 1792, 1793, 1794 targeting exon 7 used primers IL4R-5F and IL4R-8R. Those treated with SSOs 1961 and 1962 targeting exon 8 used primers IL4R-7F and IL4R-10/11R. PCR of GAPDH transcripts using primers GAPDH-F and GAPDH-R were performed as control. The lanes marked with x are the four SSO that do not show to be efficacious in inducing skipping of their target exon. The primer set used is indicated on each of the gel electrophoresis panel and listed in Table 2.

FIG. 4 shows SSO efficiency in inducing target exon skipping measured by genescan analysis. The genescan data are presented as percentage spliced in (PSI) i.e. percentage of transcripts that still retain the target exon, thus, the lower the PSI the more efficient the SSO is. Each SSO was validated at concentration of 50 nM except for SSOs 1793 and 1961 were at 100 nM. Genescan analyses were performed with primer set, IL4R-3F (in Exon 3) and IL4R-8R (in exon 8) to measure the efficiency of SSOs 1571, 1790, 1572, 1791 and 1792, and primer set, IL4R-6F (in Exon 6) and IL4R-10/11R (exons 10 & 11 junction) to measure for the efficiency of SSOs 1793, 1961 and 1962. PSI of each target exon by each SSO are presented as a bar chart. A) Exon 4 PSI before and after treated with SSO 1571 or 1790. B) Exon 5 PSI before and after treated with SSO 1572, and exon 6 PSI before and after treated with SSO 1791. C) Exon 7 PSI before and after treated with SSO 1792 or 1793. D) Exon 8 PSI before and after treated with SSO 1961 or 1962.

FIG. 5 shows SSO efficiency in downregulation of total IL-4Rα transcripts abundance measured by qPCR. HaCaT cells were treated with 50 nM of SSO unless otherwise specified, without CHX. qPCR was performed using primer set IL4R-8F (qPCR) and IL4R-9R (qPCR) to analyse the efficiency of SSOs 1571, 1572, 1789, 1790, 1791, 1792, 1793 and 1794 (top chart). A separate qPCR was performed using primer set IL4R-11F (qPCR) and IL4R-11R (qPCR) to analyse SSOs 1571, 1572, 1790, 1792, 1794, 1961 and 1962 (bottom chart).

FIG. 6 shows dose response of SSO 1962 in inducing exon 8 skipping analysed by Genescan, and plotted as exon 8 PSI, using primer set IL4R-7F and IL4R-11R. To obtain these data the transfections were done in the presence of CHX. Cells were treated with SSO at concentrations 12.5 nM, 25 nM, 50 nM and 100 nM. NC2, a scrambled-sequence SSO was used as a negative control at a concentration of 100 nM.

FIG. 7 shows the efficiency of exon 8 skipping (exon 8 PSI) induced by SSO 1962 modified entirely with either 2′-MOE- or 2′-OMe, left panel and right panel, respectively. Cells were transfected with SSO 1962 at concentrations 12.5 nM, 25 nM, 50 nM and 100 nM in the presence of cycloheximide (CHX). Genescan PCR was performed using primer set IL4R-7F and IL4R-11R.

FIG. 8 shows dose response of 2′-OMe modified SSOs 1961 and 1962 in downregulating total IL-4Rα transcripts abundance measured by qPCR. HaCaT cells were treated with SSO at concentrations 25 nM, 50 nM, 100 nM and 200 nM for 24 hours without CHX. qPCR was performed using primer set IL4R-11F (qPCR) and IL4R-11R (qPCR). HPRT1 gene was used as internal control.

FIG. 9 shows the efficacy of SSOs 1961 and 1962 in downregulating IL-4Rα analysed by Western Blot. Cells were treated with SSO 2′-OMe modified SSO 1961 (lane 3), 2′-MOE modified SSO 1961 (lane 4), 2′-OMe modified SSO 1962 (lane 5) and 2′-MOE modified SSO 1962 (lane 6) with concentration of 50 nM, without CHX, for 48 hours. NC2, SSO with scramble sequence, was used as control.

FIG. 10 shows the start and end positions of the binding site of each SSO (listed in Tables 4 and 5) relative to its target exon splice sites. Start position upstream of the acceptor splice site is given as the “negative” number of bases from the acceptor splice site. Conversely, end position downstream of the donor splice site is given as the number of bases from the donor splice site prefixed with “+”.

The invention is described in greater detail below.

### DETAILED DESCRIPTION OF THE PRESENT INVENTION

In one aspect of the present invention, there is provided a splice-switching oligonucleotide (SSO) that binds to a IL-4Rα pre-mRNA, wherein the SSO comprises a sequence selected from the group consisting of SEQ ID NOs 1 to 8.

Table 1 shows the isoforms of IL-4Rα gene according to Ensembl. The gene has 19 transcripts (splice variants).

TABLE 1

| Transcript ID | Name | bp | Protein | Biotype | CCDS | UniProt | Flags |
|---|---|---|---|---|---|---|---|
| ENST00000395762.7 | IL4R- | 3624 | 825aa | Protein | CCDS10629 | P24394-1 | MANE Select, |
| | 202 | | | coding | | | Ensembl |
| | | | | | | | Canonical, |
| | | | | | | | GENCODE |
| | | | | | | | basic, APPRIS |
| | | | | | | | P1, TSL: 1, |
| ENST00000543915.6 | IL4R- | 3539 | 825aa | Protein | CCDS10629 | P24394-1 | GENCODE |
| | 203 | | | coding | | | basic, APPRIS |
| | | | | | | | P1, TSL: 1, |
| ENST00000170630.6 | IL4R- | 3380 | 810aa | Protein | CCDS58441 | P24394-3 | GENCODE |
| | 201 | | | coding | | | basic, TSL: 5, |
| ENST00000565179.5 | IL4R- | 579 | 193aa | Protein | — | I3L4W9 | TSL: 3, CDS 5′ |
| | 212 | | | coding | | | and 3′ |
| | | | | | | | incomplete, |
| ENST00000563002.5 | IL4R- | 541 | 120aa | Protein | — | J9JII2 | TSL: 5, CDS 3′ |
| | 208 | | | coding | | | incomplete, |
| ENST00000562968.1 | IL4R- | 531 | 49aa | Protein | — | H3BSE7 | TSL: 3, CDS 5′ |
| | 207 | | | coding | | | incomplete, |
| ENST00000566117.5 | IL4R- | 529 | 120aa | Protein | — | J9JII2 | TSL: 5, CDS 3′ |
| | 216 | | | coding | | | incomplete, |
| ENST00000562142.5 | IL4R- | 518 | 63aa | Protein | — | H3BTD9 | TSL: 3, CDS 5′ |
| | 206 | | | coding | | | incomplete, |
| ENST00000565352.1 | IL4R- | 424 | 133aa | Protein | — | I3L4H7 | TSL: 5, CDS 3′ |
| | 213 | | | coding | | | incomplete, |
| ENST00000561742.5 | IL4R- | 379 | 10aa | Protein | — | A0A0G2JLA1 | TSL: 3, CDS 5′ |
| | 204 | | | coding | | | incomplete, |
| ENST00000628578.1 | IL4R- | 201 | 66aa | Protein | — | Q5FC08 | GENCODE |
| | 219 | | | coding | | | basic, TSL: 1, |
| ENST00000568746.5 | IL4R- | 2900 | 66aa | Nonsense | — | Q5FC08 | TSL: 2, |
| | 218 | | | mediated | | | |
| | | | | decay | | | |
| ENST00000566318.5 | IL4R- | 688 | 124aa | Nonsense | — | H3BRH7 | TSL: 3, |
| | 217 | | | mediated | | | |
| | | | | decay | | | |
| ENST00000563926.5 | IL4R- | 594 | 66aa | Nonsense | — | Q5FC08 | TSL: 4, |
| | 211 | | | mediated | | | |
| | | | | decay | | | |
| ENST00000565915.5 | IL4R- | 541 | No | Protein | — | — | TSL: 4, |
| | 215 | | protein | coding | | | |
| | | | | CDS not | | | |
| | | | | defined | | | |
| ENST00000563886.1 | IL4R- | 734 | No | Retained | — | — | TSL: 2, |
| | 210 | | protein | intron | | | |
| ENST00000563787.1 | IL4R- | 701 | No | Retained | — | — | TSL: 3, |
| | 209 | | protein | intron | | | |
| ENST00000561946.5 | IL4R- | 611 | No | Retained | — | — | TSL: 3, |
| | 205 | | protein | intron | | | |
| ENST00000565696.1 | IL4R- | 533 | No | Retained | — | — | TSL: 3, |
| | 214 | | protein | intron | | | |

In one embodiment, the SSO as described herein is between 18 and 32 nucleotides in length.

By “oligonucleotide”, it is meant to refer to any polynucleotide. A “polynucleotide” is an oligomer comprised of nucleotides. A polynucleotide may be comprised of DNA, RNA modified forms thereof, or a combination thereof. The term “nucleotide” or its plural as used herein is interchangeable with modified forms as discussed herein and otherwise known in the art. In certain instances, the art uses the term “nucleobase” which embraces naturally occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C [3]-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al, U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al), in Chapter 15 by Sanghvi, in Antisense Research and Application, Ed. S. T. Crooke and B. Lebleu, CRC Press, 1993, in Englisch et al, 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which is hereby incorporated by reference in its entirety). In various embodiments, polynucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles {e.g., 5-nitroindole), and optionally substituted hypoxanthine. Other desirable universal bases include pyrrole, and diazole or triazole derivatives, including those universal bases known in the art.

Polynucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include, without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (IH-pyrimido[5,4-b][1,4]benzoxazin-2 (3H)-one), phenothiazine cytidine (IH-pyrimido[5,4-b][I,4]benzothiazin-2 (3H)-one), G-clamps such as a substituted phenoxazine cytidine (e.g. 9-(2-aminoethoxy)-H-pyrimido[5,4-b][1,4]benzox-azin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al, 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity of the polynucleotide and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2. deg. C. and are, in certain embodiments, combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Modified polynucleotides are contemplated for use wherein both one or more sugar and/or one or more internucleotide linkage of the nucleotide units in the polynucleotide is replaced with “non-naturally occurring” sugars (i.e., sugars other than ribose or deoxyribose) or internucleotide linkages, respectively. In one embodiment, this disclosure contemplates a peptide nucleic acid (PNA). In PNA compounds, the sugar-backbone of a polynucleotide is replaced with an amide-containing (e.g., peptide bonds between N-(2-aminoethyl)-glycine units) backbone. See, for example U.S. Pat. Nos. 5,539,082; 5,714,331; and 5,719,262, and Nielsen et al, Science, 1991, 254, 1497-1500, the disclosures of which are herein incorporated by reference. Modified polynucleotides may also contain one or more substituted sugar groups. In one embodiment, a modification of the sugar includes Locked Nucleic Acids (LNAs) in which the 2′-hydroxyl group is linked to the 3′ or 4′ carbon atom of the sugar ring, thereby forming a bicyclic sugar group. The linkage is in certain embodiments a methylene (—CH_{[2]}—)_{[n]} group bridging the 2′ oxygen atom and the 4′ carbon atom wherein n is 1 or 2. LNAs and preparation thereof are described in WO 98/39352 and WO 99/14226, the disclosures of which are incorporated herein by reference. In the present invention, preferably, the antisense oligonucleotide comprises a modified polynucleotide backbone. The modified polynucleotide backbone may comprise a modified moiety substituted for the sugar of at least one of the polynucleotides. The modified moiety may be selected from the group comprising of phosphorodiamidate morpholino oligomer (PMO), peptide-conjugated phosphorodiamidate morpholino oligomer (PPMO), and non-peptide dendrimeric octaguanidine moiety-tagged morpholino oligomer.

In various embodiments, the modified polynucleotide backbone comprises at least one modified internucleotide linkage. The modified internucleotide linkage comprises a modified phosphate. The modified phosphate may be any one selected from the group comprising of a non-bridging oxygen atom substituting a sulfur atom, a phosphonate, a phosphorothioate, a phosphodiester, a phosphoromorpholidate, a phosphoropiperazidate and a phosphoroamidate.

In various embodiments of the invention, the SSO comprises a backbone selected from the group comprising of ribonucleic acid, deoxyribonucleic acid, DNA phosphorothioate, RNA phosphorothioate, 2′-O-methyl-oligoribonucleotide and 2′-O-methyl-oligodeoxyribonucleotide, 2′-O-hydrocarbyl ribonucleic acid, 2′-O-hydrocarbyl DNA, 2′-O-hydrocarbyl RNA phosphorothioate, 2′-O-hydrocarbyl DNA phosphorothioate, 2′-F-phosphorothioate, 2′-F-phosphodiester, 2′-methoxyethyl phosphorothioate, 2-methoxyethyl phosphodiester, deoxy methylene (methylimino) (deoxy MMI), 2′-O-hydrocarby MMI, deoxy-methylphos-phonate, 2′-O-hydrocarbyl methylphosphonate, morpholino, 4′-thio DNA, 4′-thio RNA, peptide nucleic acid, 3′-amidate, deoxy 3′-amidate, 2′-O-hydrocarbyl 3′-amidate, locked nucleic acid, cyclohexane nucleic acid, tricycle-DNA, 2′fluoro-arabino nucleic acid, N3′-P5′ phosphoroamidate, carbamate linked, phosphotriester linked, a nylon backbone modification and mixtures of the aforementioned backbones.

Preferably, the oligonucleotide is chemically linked to one or more conjugates that enhance the activity, tissue/organ distribution, or cellular uptake of the SSO.

As used herein, the term “splice switching oligonucleotides” (SSOs) or “splice switching oligomers” is meant to include synthetic antisense nucleic acids that base-pair with a pre-mRNA and disrupt the splicing process by sterically blocking the RNA-RNA base-pairing or protein-RNA binding interactions that occur between components of the splicing machinery and the pre-mRNA. The SSOs may also be known as “antisense nucleotides” which can modulate splicing. SSOs may modulate splicing via steric blocking. In some embodiments, SSOs may be mixmers. The term “mixmer” includes an oligomer on which different types of chemical modifications are applied on its sugar moieties, or on its backbone linkages, or both. Examples of chemical modifications include phosphorothioate linkages, 2′-O-methyl RNA modifications, 2′-O-methoxyethyl RNA modifications and locked nucleic acid substitutions. The terms “phosphorothioate bond” and “phosphorothioate linkage” are used interchangeably. The chemical modifications may increase the efficacy, selectivity and stability while manifesting superior toxicity profile of SSOs.

The term “splicing” refers to an RNA processing mechanism in which a pre-mRNA is made into a mature mRNA. During splicing, introns are removed and exons are connected. Splicing is catalysed by the spliceosome complex. As used herein, the term “alternative splicing” is meant to include a process by which a gene can encode for multiple mRNA and protein products by differentially selecting which exons are to be included in a mature mRNA transcript. For example, alternative splicing can take the form of one or more skipped exons, variable position of intron splicing, or intron retention.

As used herein, the term “intron” refers to a segment of non-coding nucleic acid sequence that is transcribed and is present in the pre-mRNA but is excised by the splicing machinery and therefore not present in the mature mRNA transcript.

As used herein, the term “exon” refers to a segment of a nucleic acid sequence that is transcribed into mRNA and that is present in mature mRNA after splicing. The term “exon skipping” is meant to include the process by which an entire exon, or a portion thereof, is removed from a given pre-mRNA and is thereby excluded from being present in the mature mRNA. For example, the portion of the protein that is otherwise encoded by the skipped exon is not present in the expressed form of the protein.

As used herein, the term “splice site” is meant to include specific nucleic acid sequences that can be recognized by the splicing machinery as being suitable for excision and/or ligation with the corresponding splice site. The splice site defines the precise exon-intron boundary that allows the excision of introns present in pre-mRNA transcripts. As used herein, the term “5′ splice site” (also known as donor splice site) refers to a nucleic acid sequence surrounding the exon-intron boundary at the 5′ end of an intron that marks the start of the intron and its boundary with the preceding exon sequence. The term “3′ splice site” (also known as acceptor splice site) as used herein refers to a nucleic acid sequence surrounding the intron-exon boundary at the 3′ end of an intron that marks the end of the intron and its boundary with the following exon sequence.

As used herein, the term “pre-mRNA” or “precursor mRNA” refers to a strand of messenger ribonucleic acid (mRNA), synthesized from a DNA template by transcription. Pre-mRNA is composed of exons, introns and untranslated sequences (before the first and after the last exons respectively). Generally, eukaryotic pre-mRNA exists only briefly before it is fully processed into mature mRNA.

The term “binding” as used in the context of an SSO is meant to include the hybridization of the SSO to a site on a pre-mRNA or mature mRNA transcript. The term “hybridize” or “hybridization” may include the binding of a single-stranded nucleic acid or a locally single-stranded region of a double-stranded nucleic acid to another single-stranded nucleic acid or a locally single-stranded region of a double-stranded nucleic acid having a complementary sequence through the pairing of complementary nucleic acids. It is generally known to a person skilled in the art that binding or hybridization of one sequence to another does not require total complementarity of the sequences. For example, the sequence of the SSO may be completely complementary or partially complementary to the site to which it binds.

Advantageously, the SSOs of the present embodiments are able to bind to the respective binding sites on the IL-4Rα pre-mRNA competitively due to favourable binding thermodynamics and extent of locally single-stranded binding site identified. Selection of the binding site involves considering the presence of RNA-binding protein motifs on the binding site. The SSOs of the present embodiments are able to induce the desired splicing modulation by blocking the appropriate RNA-binding protein(s).

In another embodiment, at least one of the nucleotides of the SSO as described herein is chemically modified. The chemical modification may be a 2′-O-methyl RNA modification, 2′-O-methoxyethyl RNA modification or phosphorothioate linkage.

In one embodiment, each nucleotide of the SSO as described herein comprises either a 2′-O-methyl RNA modification or a 2′-O-methoxyethyl RNA modification.

In another embodiment, the SSO as described herein comprises phosphorothioate linkages between all nucleotides of the SSO.

In another aspect of the invention, there is provided the SSOs of this invention for use as a medicament or in therapy. In various embodiments, there is provided an SSO as described herein for use in the treatment of a Th2-mediated inflammatory disease. In view of the role of IL-4Rα in IL-4 and IL-13 signalling, which are key mediators of the Th2 response, it would be generally understood by a person skilled in the art that a reduction in IL-4Rα expression would lead to a reduction in the Th2 immune response, thereby alleviating Th2-mediated inflammatory diseases. Examples of Th2-mediated inflammatory diseases are atopic diseases that include but are not limited to atopic dermatitis (also known as eczema), asthma, allergic rhinitis (also known as hay fever) and allergic conjunctivitis, as well as ulcerative colitis, a form of inflammatory bowel disease that is caused by Th2 immune response. Atopic dermatitis, asthma and allergic rhinitis together are called atopic triad as an individual with atopy often experience all three conditions. It is usually started with atopic dermatitis in the infancy followed by asthma and allergic rhinitis later in the childhood. Allergic conjunctivitis is comprised of chronic allergic conjunctivitis (CAC) that includes atopic kerato-conjunctivitis (AKC), perennial allergic conjunctivitis (PAC) and vernal kerato-conjunctivitis (VKC), as well as seasonal acute allergic conjunctivitis (SAC). Allergic conjunctivitis may appear as SAC or CAC. AKC, the majority form of CAC, usually has onset in the 20th year to the 30th year of life. Among AKC cases 95% have AD history while asthma and allergic rhinitis each is linked to 65-87% of AKC. Atopy is a genetic predisposition with tendency of excessive response immunologically to otherwise harmless substance, via CD4+ Th2 differentiation and overproduction of immunoglobulin E (IgE).dermatitis. In another aspect, there is provided a use of an SSO as described herein in the manufacture of a medicament for treating a Th2-mediated inflammatory disease. In one embodiment, the Th2-mediated inflammatory diseases selected from the group consisting of atopic dermatitis, asthma, allergic rhinitis, allergic conjunctivitis and ulcerative colitis. In yet another aspect, there is provided a method of treating a Th2-mediated inflammatory disease comprising administering to a subject a composition comprising an SSO as described herein. In one embodiment, the Th2-mediated inflammatory disease is selected from the group consisting of atopic dermatitis, asthma, allergic rhinitis, allergic conjunctivitis and ulcerative colitis.

As used herein, the term “treat” or “treating” in the context of treating a disease such as a atopic dermatitis is meant to include improving clinical condition of patients having the disease. This includes reducing the severity and preventing or slowing the progression of the disease.

These SSOs may be used in compositions that can be used for treatment, e.g. as a pharmaceutical composition comprising the SSO of the invention and a pharmaceutically acceptable carrier. The composition is suitable for parenteral administration either naked or complexed with a delivery agent to a patient. The carrier is selected from the group consisting of a nanoparticle, such as a polymeric nanoparticle; a liposome, such as pH-sensitive liposome, an antibody conjugated liposome; a viral vector, a cationic lipid, a polymer, a UsnRNA, such as U7 snRNA and a cell penetrating peptide. The SSO is administered topically, or orally, or rectal, or transmucosal, or intestinal, or intramuscular, or subcutaneous, or intramedullary, or intrathecal, or direct intraventricular, or intravenous, or intravitreal, or intraperitoneal, or intranasal, or intraocular.

In another aspect of the present invention, there is provided a pharmaceutical composition comprising (a) a therapeutically effective amount of an SSO as described herein and (b) one or more pharmaceutically acceptable carriers and/or diluents. In one embodiment, the pharmaceutical composition may further comprise one or more moisturising ingredients or anti-itch ingredients. In one embodiment, the pharmaceutical composition is applied to affected skin once or twice daily.

The term “therapeutically effective amount” refers to the amount of the SSO as described herein that is required to confer the intended therapeutic effect in the subject, which amount will vary depending on the route of administration, status of disease, body weight and possible inclusion of other therapeutics or excipients. A “therapeutically effective amount” refers to an amount effective, at dosages and for periods of time necessary, to achieve a desired therapeutic result. A therapeutically effective amount may vary according to factors such as the disease state, width of the organ, e.g. affected skin area, age, sex, and weight of the individual, and the ability of the therapeutic agent to elicit a desired response in the individual.

A “therapeutically effective amount” for therapy may also be measured by its ability to stabilize the progression of disease. A therapeutically effective amount of a therapeutic agent may reduce or ameliorate symptoms in a subject. One of ordinary skill in the art would be able to determine such amounts based on such factors as the subject's size, the severity of the subject's symptoms, and the particular composition or route of administration selected.

In the methods of the invention, therapy is used to provide a positive therapeutic response with respect to a disease or condition. The term “positive therapeutic response” is intended to include an improvement in the disease or condition, and/or an improvement in the symptoms associated with the disease or condition, and/or prevent the worsening of symptoms associated with the disease or condition. Positive therapeutic responses in any given disease or condition can be determined by standardized response criteria specific to that disease or condition. In addition to these positive therapeutic responses, the subject undergoing therapy may experience the beneficial effect of an improvement in the symptoms associated with the disease.

A pharmaceutically acceptable carrier refers, generally, to materials that are suitable for administration to a subject wherein the carrier is not biologically harmful, or otherwise, causes undesirable effects. Such carriers are typically inert ingredients of a medicament. Typically a carrier is administered to a subject along with an active ingredient without causing any undesirable biological effects or interacting in a deleterious manner with any of the other components of a pharmaceutical composition in which it is contained. Suitable pharmaceutical carriers are described in Martin, Remington's Pharmaceutical Sciences, 18th Ed., Mack Publishing Co., Easton, Pa., (1990), incorporated by reference herein in its entirety.

In a more specific form of the disclosure there are provided pharmaceutical compositions comprising therapeutically effective amounts of an SSO together with pharmaceutically acceptable diluents, preservatives, solubilizers, emulsifiers, adjuvants and/or carriers. Such compositions include diluents of various buffer content (e.g., phosphate, Tris-HCl, acetate), pH and ionic strength and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as, for example and without limitation, polylactic acid or polyglycolic acid, or into liposomes. Hyaluronic acid (HA) may also be used. Such compositions may influence the physical state, stability, rate of in vivo release, and rate of in vivo clearance of the disclosed compositions.

The compositions may be prepared in liquid form, or may be in dried powder, such as lyophilized form.

It will be appreciated that pharmaceutical compositions provided according to the disclosure may be administered by any means known in the art. Preferably, the pharmaceutical compositions for administration are administered by injection, topically, or orally, or by the pulmonary, or nasal route. The antisense polynucleotides are, in various embodiments, delivered by intravenous, intra-arterial, intraperitoneal, intramuscular, transdermal, or subcutaneous routes of administration.

In one embodiment, the pharmaceutical compositions comprising an SSO as described herein may be delivered via microneedle that include self-dissolving microneedle patch and microneedle. Self-dissolving microneedle patch is made of HA that is casted into crystal in the form of microneedle. SSO can be embedded by mixing it with HA solution before to be casted into microneedle patch. Once the microneedles puncture through the skin barrier, they will naturally melt releasing the SSO for maximum uptake by the cells in the epidermis and dermis while the HA is being absorbed by the skin tissue. Microneedle devices which are made of plastics or metal or other polymer can also be used to deliver substances through the skin to the body. A microneedle device may be used to deliver a drug directly to the epidermis and dermis. The microneedles puncture the epidermal barrier and deliver the drug directly to the epidermis, dermis and even further diffuse to the bloodstream and absorption by the body's active tissues. The microneedle patch will be attached to a reservoir that will contain the composition to be delivered, and this reservoir will itself be attached to contain an apparatus to encourage flow of the composition contained in the reservoir through the microneedles and into the skin. The above delivery methods have advantage over oral delivery because it allows the composition to be absorbed into the body without coming into contact with powerful digestive enzymes of the stomach, over injectable delivery because it is comparatively painless, and over topical delivery because it allows for much greater absorption of the delivered composition.

The pharmaceutical compositions comprising an SSO as described herein may also be delivered via lipid nanoparticles. Lipid nanoparticles as used herein may refer to carrier systems in the nanometer size comprising a continuous aqueous phase and at least one dispersed oily phase, in which the oily phase comprises at least one amphiphilic lipid such as phospholipids and at least one solubilizing lipid with a monolayer around an amorphous core. Lipid nanoparticles are known for their high degree of biocompatibility, controlled release, efficient targeting, stability, natural biodegradability and high therapeutic index to their payload. Lipid nanoparticles may be assembled as solid lipid nanoparticles (SLN), nanostructured lipid carriers (NLC), and NanoSpheres (NS). The lipids used in the process of synthesizing the lipid nanoparticle compositions may include fatty acids, triglycerides, triacylglycerols, acylglycerols, fats, waxes, cholesterol, sphingolipids, glycerides, sterides, cerides, glycolipids, sulfolipids, lipoproteins, chylomicrons and the derivatives of these lipids. Surfactants used in the assembly of lipid nanoparticles may include biocompatible and biodegradable surfactants such as lecithins, polysorbates, monoglycerides, diglycerides, triglycerides, glyceryl oleate, polaxamers and other non-toxic, non-ionic surfactants that are known in the art.

The oligonucleotides of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

For polynucleotides, preferred examples of pharmaceutically acceptable salts include, but are not limited to, (a) salts formed with cations such as sodium, potassium, ammonium, magnesium, calcium, polyamines such as spermine and spermidine; (b) acid addition salts formed with inorganic acids, for example hydrochloric acid, hydrobromic acid, sulfuric acid, phosphoric acid, nitric acid; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid; and (d) salts formed from elemental anions such as chlorine, bromine, and iodine. The pharmaceutical compositions of the disclosure may be administered in a number of ways depending upon whether local or systemic treatment is desired and upon the area to be treated. Administration may be topical (e.g. lotion/cream/microneedle based, ophthalmic or to mucous membranes including rectal delivery), pulmonary, e.g., by inhalation of powders or aerosols (including by nebulizer, intratracheal, intranasal), oral or parenteral. Parenteral administration includes intravenous, intra-arterial, subcutaneous, transdermal, intraperitoneal or intramuscular injection or infusion; or intracranial, e.g., intrathecal or intraventricular, administration.

The pharmaceutical formulations of the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly bringing into association the active ingredients with liquid carriers or finely divided solid carriers or both, and then, if necessary, shaping the product.

Combination therapy with an additional therapeutic agent may also be contemplated by the disclosure.

In some embodiments, the present invention may be used in gene therapy such as, e.g. using a vector (e.g., an expression vector) comprising a polynucleotide of the invention to direct expression of the polynucleotide in a suitable host cell. Such vectors are useful, e.g., for amplifying the polynucleotides in host cells to create useful quantities thereof. In some embodiments, the vector is an expression vector wherein a polynucleotide of the invention is operatively linked to a polynucleotide comprising an expression control sequence.

In an aspect of the present invention, there is provided a method of exon-skipping comprising providing an SSO having a sequence selected from the group consisting of SEQ ID NOs 1 to 8, wherein the binding of the SSO to a IL-4Rα pre-mRNA induces the exclusion of an exon during splicing of the IL-4Rα pre-mRNA to a IL-4Rα mature mRNA.

“IL-4Rα” as used herein refers to the subunit of interleukin-4 and interleukin-13 receptors.

The exon to be excluded may also be known as “target exon”.

In one embodiment, the SSO has a binding site that lies within the target exon. In another embodiment, the SSO has a binding site that overlaps with the acceptor splice site of the target exon and with or a part of the target exon. In yet another embodiment, the SSO has a binding site that overlaps with or a part of the target exon and with the donor splice site of the target exon.

In one embodiment, the exclusion of the exon in the method as described herein causes a reduction in the levels of the IL-4Rα mature mRNA or functional protein. This reduction in the levels of the IL-4Rα mature mRNA may result in a loss of IL-4Rα protein expression.

In the method as described herein, the exon to be excluded comprises a number of nucleotides not divisible by 3. The exclusion of this exon may result in a shift in the codon reading frame. Whether a decrease in the levels of IL-4Rα mRNA transcript is observed would depend on the efficiency of degradation of these mRNA transcripts via the nonsense-mediated decay (NMD) pathway. It is generally known in the art that the propensity and efficiency of NMD can vary among different genes and among transcripts expressed from a gene, and among cell types and tissues. Nevertheless, since the method of exon-skipping of the present invention results in a shift in codon reading frame, the resultant mRNA transcripts would likely manifest multiple premature termination codons downstream of the skipped exon. These transcripts, if ever translated, will produce non-functional truncated proteins. Even if premature termination codons are not presented downstream of the skipped exon, the resultant peptide sequence would vary significantly from the wild-type and therefore the resultant protein structure would be misfolded and hence non-functional.

In the method as described herein, the exon to be excluded is selected from the group consisting of exon 2, exon 3, exon 4, exon 5, exon 6, exon 7 and exon 8 of IL-4Rα pre-mRNA (Genbank Gene ID: 3566; Ensembl: ENSG00000077238) (SEQ ID NO: 38).

For example, the exclusion of exon 8, which comprises 100 nucleotides and encodes a transmembrane domain of IL-4Rα protein, may result in one or more of the following consequences. Firstly, the exclusion of exon 8 may cause the disruption of IL-4Rα mRNA reading frame, followed by premature stop codons that may result in nonsense-mediated decay of the mRNA and thus a reduction in IL-4Rα protein expression. Secondly, the exclusion of exon 8 may cause the transmembrane domain to be absent from the IL-4Rα protein. For example, the skipping of exon 8 may be accompanied by other alternative splicing events such as the skipping of other exons or retention of partial introns that would allow a transcript without exon 8 to still maintain the correct reading frame. This transcript may code for a protein, but the absence of the transmembrane domain from the protein will render the protein incapable of localising itself as a transmembrane protein. Such a protein cannot play a role in the signalling pathway that is crucial for the development and exacerbation of AD.

In another embodiment, the method as described herein comprises providing an SSO with at least one of the nucleotides of the SSO being chemically modified and wherein the chemical modification is 2′-O-methyl RNA modification, 2′-O-methoxyethyl RNA modification or phosphorothioate linkage.

In another embodiment, the method as described herein comprises providing an SSO in which each nucleotide of the SSO comprises either a 2′-O-methyl RNA modification or a 2′-O-methoxyethyl RNA modification.

In another embodiment, the method as described herein comprises providing an SSO comprising phosphorothioate linkages between all nucleotides of the SSO.

The listing or discussion of an apparently prior-published document in this specification should not necessarily be taken as an acknowledgement that the document is part of the state of the art or is common general knowledge.

Any document referred to herein is hereby incorporated by reference in its entirety.

In order that the present invention may be fully understood and readily put into practical effect, there shall now be described by way of non-limitative examples only preferred embodiments of the present invention, the description being with reference to the accompanying illustrative figures.

### MATERIALS AND METHODS

### Cells

HaCaT cells (RRID:CVCL_0038) was used in the experiments.

Transfection of SSO into HaCaT Cells

Each SSO was tested and validated in HaCaT cells for their efficacy and efficiency. Transfections were performed on 12-well cell culture plates with cell confluency between 50% to 75%. In separate tubes, SSO with indicated concentration and an indicated amount of Lipofectamine RNAiMAX, hereafter called lipofectamine, were diluted in serum-free antibiotic-free Opti MEM medium so that the volume of each tube was 25 ul. After 5 minutes, the two diluted compounds were to be mixed in one tube and left at room temperature for 15 minutes to form SSO-lipofectamine complexes. After 15 minutes, 950 ul growth medium without antibiotics was added into the 50 ul SSO-Lipofectamine complex, after which the transfection mixture is ready to be used. Growing medium on the cells was removed and replaced with the transfection mixture and the cells were left in CO₂ incubator for 24 hours to be ready for analyses. Depending on the experiments, five hours after the application of transfection mixture, cycloheximide (CHX) of 10 μg/ml might be added. A scrambled-sequence SSO-Lipofectamine complex and a mixture containing diluted lipofectamine only were used as controls.

### Analyses of the Efficacy and Efficiency of the SSO

Four analyses were performed to analyse the efficacy and efficiency of the SSO. Firstly, to select efficacious SSOs, after transfection, the cells were subjected to RNA isolation, Reverse-transcriptase PCR (RT-PCR), agarose gel electrophoresis and the Sanger sequencing to verify the skipping of target exon specifically. Secondly, for the SSOs shown to be efficacious, further analysis was performed by transfection followed by RT-PCR Genescan to semi-quantitate their efficiency in inducing specific exon skipping. Thirdly, RT-qPCR was also performed to measure the effect of each SSO in reducing the IL-4Rα abundance in the treated cells. Fourthly, The SSOs showed to be efficient in inducing specific exon skipping as well as IL-4Rα transcript abundance were re-synthesised in 2-Ome and 2-Moe and further analysed for their dose-response efficiency that was analysed by RT-PCR genescan as well as qPCR. Lastly, these SSOs were also analysed for their ability to downregulate IL-4Rα protein expression by Western blot.

RNA Isolation and First Strand cDNA Synthesis (Reverse Transcription)

Total RNA was isolated from the HaCaT cells using Trizol (Invitrogen, Carlsbad, CA). At indicated time, the medium containing transfection reagent was removed and the cells were washed gently using pre-warmed phosphate buffered saline (PBS). Cells were harvested by pouring Trizol buffer (500 ul for each well of 12-well plate) directly onto the cells, 10 minutes lysing in room temperature followed by homogenization using micropipette. The cells' lysate then to be collected in 1.5 ml Eppendorf tube and subjected to further total RNA isolation protocol provided by the Trizol manufacturer. First-strand cDNA was synthesized using oligo (dT) primer and Maxima First Strand cDNA Synthesis Kit (Thermo Scientific). The cDNA (RT product) then to be used for further analyses i.e. PCR followed by agarose gel electrophoresis and Sanger sequencing, PCR genescan, and qPCR.

### PCR

PCRs were carried out using DreamTaq Green PCR Master Mix (2×) (Thermo Fisher Scientific) with cDNA (of equal to 100 ng total RNA) in 25 ul reaction volume, and 10 pM of different primer sets according to the SSO (primers to encompass a region harbouring the exon and at least one exon upstream and one exon downstream targeted by the analysed SSO. PCRs were performed in Thermal Cycler C1000 Touch (Biorad) with total of 35 cycles comprised of denaturing temperature at 95° C. for 20 seconds, annealing temperature of 58° C. to 60° C. for 20 seconds and elongation temperature at 72° C. for 1 minute. The PCR products were then electrophoresed in 1% agarose gel and the amplicons to be visualised on UV table.

### Sequencing

The bands observed in the agarose gel electrophoresis were cut, separately purified using QIAquick Gel Extraction Kit (Qiagen) and subjected to Sanger sequencing. The Sanger sequencing was done by BioBasic Asia Pacific, Singapore.

### Genescan

PCRs were carried out using DreamTaq Green PCR Master Mix (2×) (Thermo Fisher Scientific) with cDNA (of equal to 125 ng total RNA) in 25 ul reaction volume, and 10 pM of indicated primer set with the forward primer to be FAM-labelled (IDT). PCR was performed in Thermal Cycler C1000 Touch (Biorad) with total of 35 cycles comprised of denaturing temperature at 95° C. for 20 seconds, annealing temperature at 58° C. for 20 seconds and elongation temperature at 72° C. for 1 minute. 1.5 ul of the PCR products was added to 10 ul HiDi Formamide (Thermo Fisher Scientific) with 1 ul size marker (GeneScan™ 500 LIZ™ Size Standard or GeneScan™ 1200 LIZ™ dye Size Standard, Thermo Fisher Scientific, depending on the expected size of the PCR products), and analyzed in the 3730xl DNA Analyzer (Thermo Fisher Scientific). The data generated were analyzed using GeneMapper™ Software (Thermo Fisher Scientific). The data were presented as percentage of spliced (PSI) i.e. percentage of transcripts that still retain the target exon, thus, the lower the PSI means the more efficient the SSO is.

Quantitative PCRs (qPCRs)

qPCRs were performed using the DNA Master SYBR green I kit (Roche Diagnostics, Basel, Switzerland) with cDNA of equal to 35 ng total RNA in 25 ul reaction volume with indicated primer set. To control the RNA integrity and for differences across tissues or differences attributable to errors in experimental manipulation from tube to tube, Hypoxanthine Guanine Phosphoribosyltransferase 1 (HPRT1) was used as endogenous control with primers HPRT1-F and HPRT1-R. The reactions were performed in Light Cycler CFX96 Real-Time System (Biorad) and the data were evaluated using corresponding software (Biorad) and presented as the relative ratio of the IL-4Rα/HPRT.

### Western Blot

HaCaT cells were grown in 6-well plate in DMEM supplemented with 10% fetal bovine serum. Cells in each well were transfected with an indicated SSO at concentration of 50 nM with Lipofectamine RNAiMAX at concentration of 2.5 μl/ml medium, without CHX. After 48 hours the cells were harvested and subjected to protein extraction and Western Blot analysis with standard protocol using antibody anti-IL-4Rα (Santa Cruz Biotechnology, Inc. Dallas, TX, USA) and anti B-actin (Santa Cruz Biotechnology, Inc. Dallas, TX, USA). 10 μg of protein was loaded in each well.

TABLE 2
List of primers used

| SEQ | | |
|---|---|---|
| ID | | |
| NO | Primer Name | Sequence |
| 13 | IL4R-1F | GGCGCGCAGATAATTAAAGA |
| 14 | IL4R-5R | AGCCCACAGGTCCAGTGTAT |
| 15 | IL4R-3F | ATGGGGTGGCTTTGCTCT |
| 16 | IL4R-7R | CGCGGGCCAGGGAAGGGCCA |
| 17 | IL4R-8R | CTCCACTCACTCCAGGTGGT |
| 18 | IL4R-5F | CTCATGGATGACGTGGTCAG |
| 19 | IL4R-6F | GACACTCTGCTGCTGACCTG |
| 20 | IL4R-10/11R | TTCTTCCAGTGTGGGCACTT |
| 21 | IL4R-7F | ACCACCTGGAGTGAGTGGAG |
| 22 | IL4R-11R | CTGGAAAGGCATCTCTTTGG |
| 23 | IL4R-8F(qPCR) | TTCCTGCATTGTCATCCTG |
| 24 | IL4R-9R(qPCR) | CTGGGTTGGGAATCTGATC |
| 25 | IL4R-11F(qPCR) | CAAGCTCTTGCCCTGTTTTC |
| 26 | IL4R-11R(qPCR) | TGCACAGAAGCTCCCTTTTT |
| 27 | HPRT1-F | TGCTGAGGATTTGGAAAGGG |
| 28 | HPRT1-R | ACAGAGGGCTACAATGTGATG |
| 29 | GAPDH-F | GCAAATTCCATGGCACCGT |
| 30 | GAPDH-R | GCCCCACTTGATTTTGGAGG |

### EXAMPLES

### Example 1

The strategy employed for the present invention was to develop SSOs to mediate the suppression of interleukin-4 receptor alpha (IL-4Rα), a component of both IL-4R type 1 (IL-4Ra/gc; IL-4 specific) and type 2 (IL-4Ra-IL-13Ra1; IL-4 and IL-13 specific). IL-4Rα has been shown to be effected by Dupilumab, a clinically validated therapeutic target in severe AD. By formulating with an appropriate lotion, one of the aims was to develop a topical therapy for a localized disease.

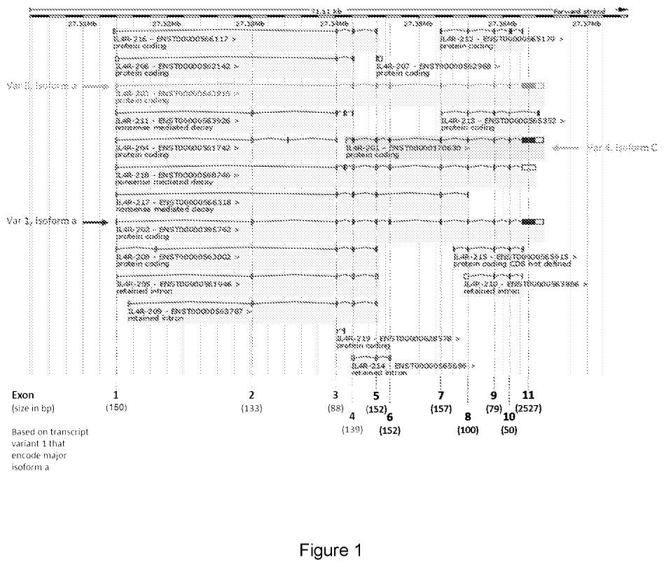

As depicted in FIG. 1, the IL-4Rα gene expresses many isoforms, which are resulted from the usage of alternative start codon, alternative splicing of exons 6 and/or 10, or alternative polyadenylation usage. As a strategy to suppress the expression of the gene, SSOs were rationally designed to induce specific exon skipping during pre-mRNA splicing, with the aim to disrupt the mRNA reading frame. As the resultant frameshifted transcripts harbor multiple premature termination codons, they will either be degraded via the nonsense-mediated decay (NMD) pathway or resulted in the failure to code for functional IL-4Rα protein. Exons whose lengths are not divisible by three are potential targets, as their individual exclusion will result in codon reading frameshift.

### Example 2

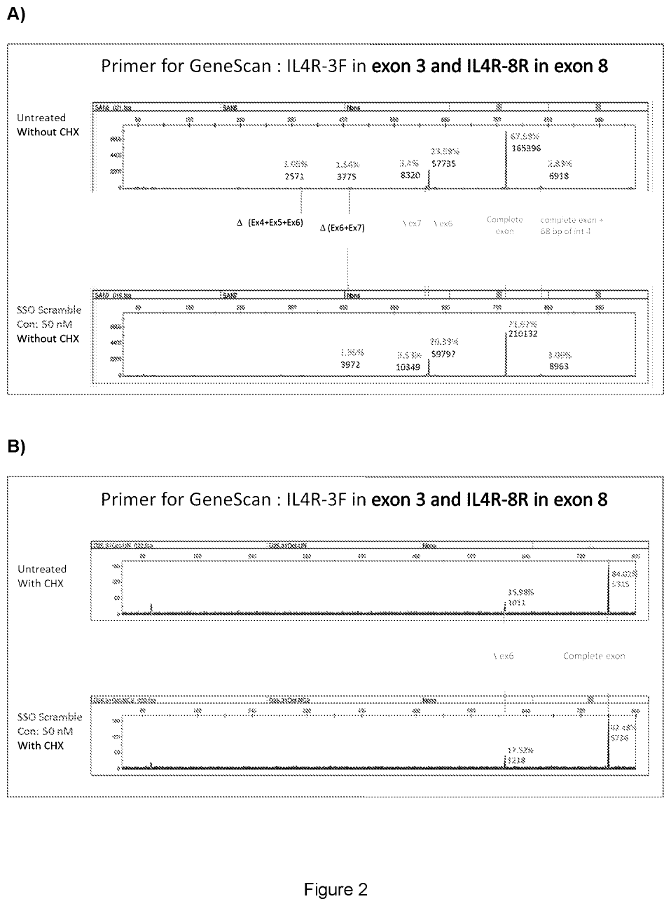

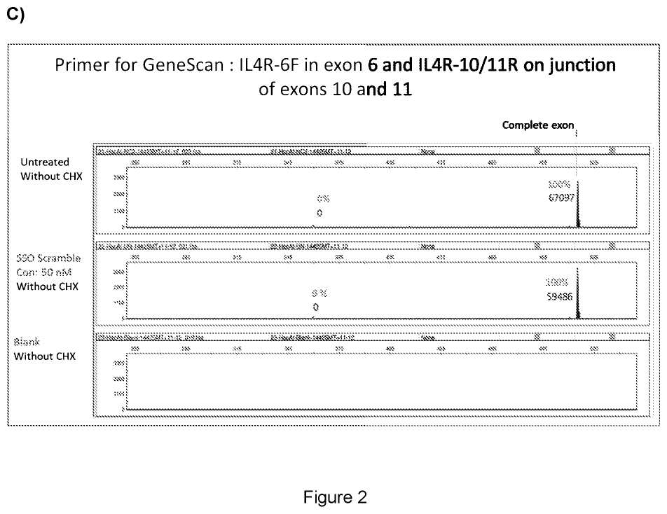

The relative abundances of the possible isoforms were characterized in the in-vitro model HaCaT cells, with or without treatments of cycloheximide (CHX) and a scrambled SSO (negative control); CHX is an inhibitor of protein translation that is essential for the activation of NMD. As shown in the data from Genescan given in FIG. 2, the major isoform includes all the exons.

### Example 3

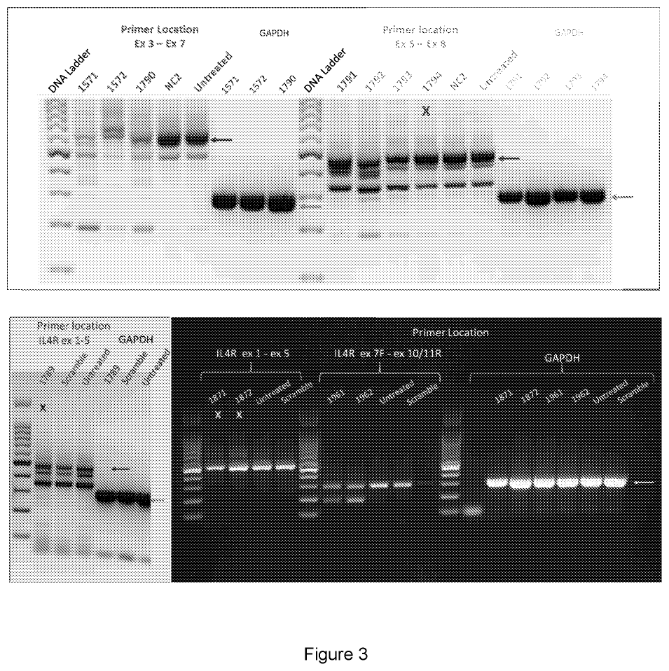

12 SSOs were rationally designed each to induce exclusion of one of the 7 possible target exons, as tabulated in Table 3. Each of the SSO was validated for its efficacy in inducing skipping of its target exon. The validation was performed by transfection of each SSO into HaCat cells and analyzed by reversed-transcriptase PCR, followed by agarose gel electrophoresis as described in the Method above. Upon agarose gel electrophoresis and sequencing verification, all but SSOs 1871, 1872, 1789 and 1794 showed bands/amplicons that were of smaller molecular weight, or reduced intensity of the band corresponding to the unskipped transcripts when compared to those untreated or Scramble SSO treated lanes. They were thus deemed to be efficacious in inducing specific target exon skipping. By contrast, SSOs 1871, 1872, 1789 and 1794 showed bands/amplicons of the same sizes and density with those untreated or Scramble SSO treated lanes, and were non-efficacious and are marked with an X. The results of the efficacy analysis are shown in FIG. 3. The eight SSOs that showed efficacy are 1571, 1790, 1572, 1791, 1792, 1793, 1961 and 1962.

TABLE 3
SSOs designed and their target exons

| SSO ID | Target Exon | Targeted exon size (bp) |
|---|---|---|
| 1871/1872 | Exon 2 | 133 |
| 1789 | Exon 3 | 88 |
| 1571/1790 | Exon 4 | 139 |
| 1572 | Exon 5 | 152 |
| 1791 | Exon 6 | 152 |
| 1792/1793/1794 | Exon 7 | 157 |
| 1961/1962 | Exon 8 | 100 |

### Example 4

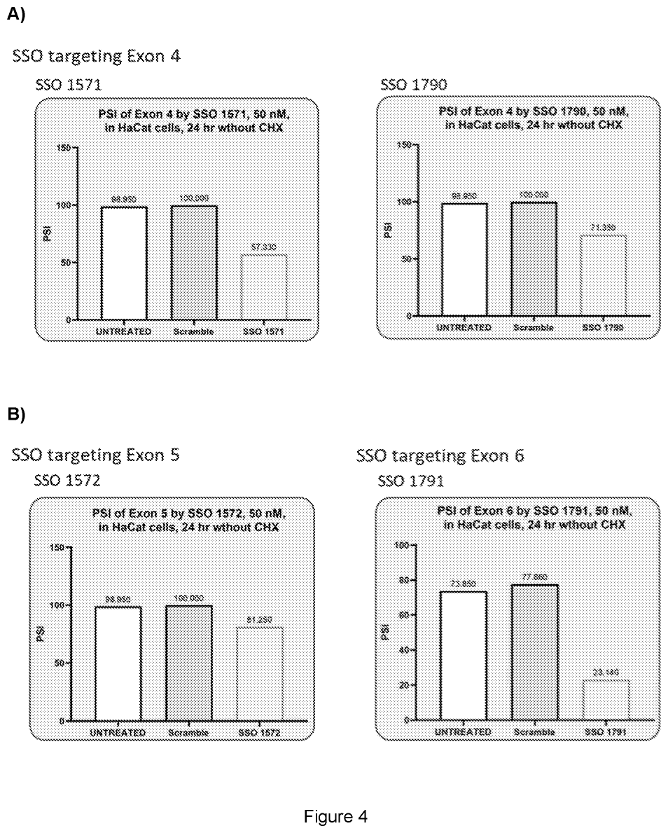

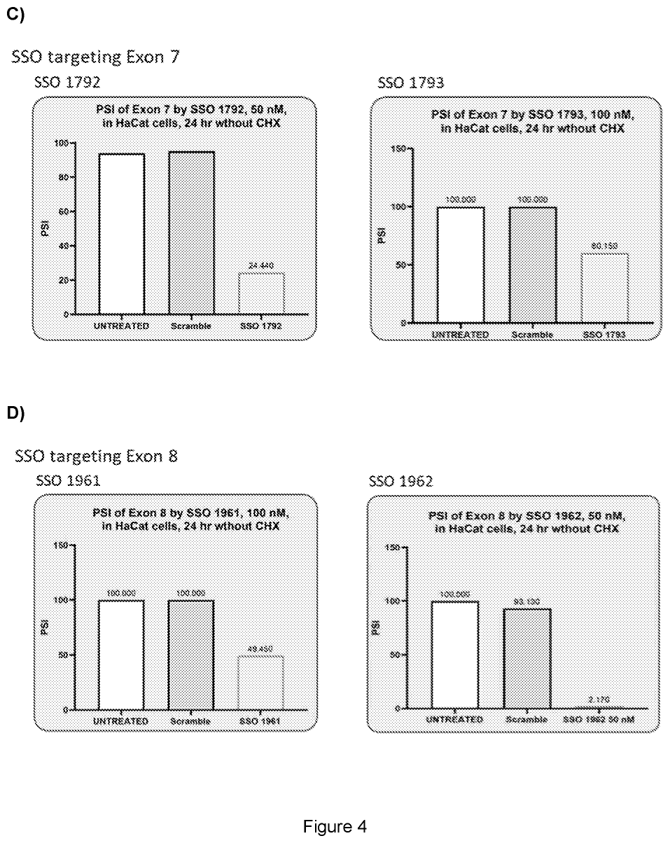

One way of quantifying the efficiency of the 8 effective SSOs is to measure the PSI for the respective target exon using Genescan. The PSI of each targeted exon from the untreated cells, cells treated with SSO of scrambled sequence as negative control and cells treated with specific SSO are presented in the bar charts (FIG. 4). The PSI score reversely reflexes the efficiency of the treatment. Four out of the eight SSOs, namely SSO 1791, 1792, 1961 and 1962, were able to achieve PSI<0.5 at 50 nM, except SSO 1961 which was able to achieve PSI<0.5 at 100 nM (FIG. 4).

### Example 5

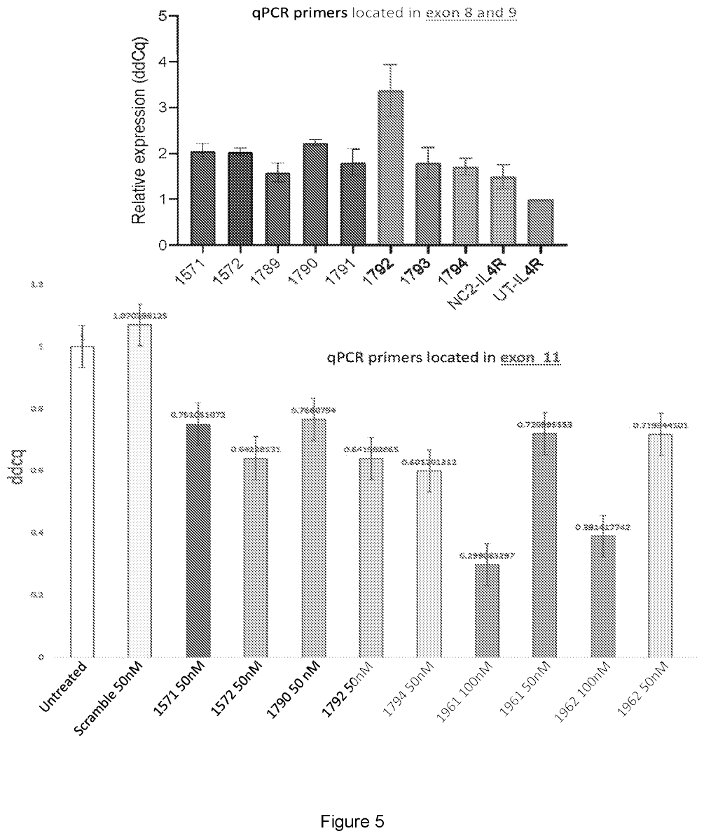

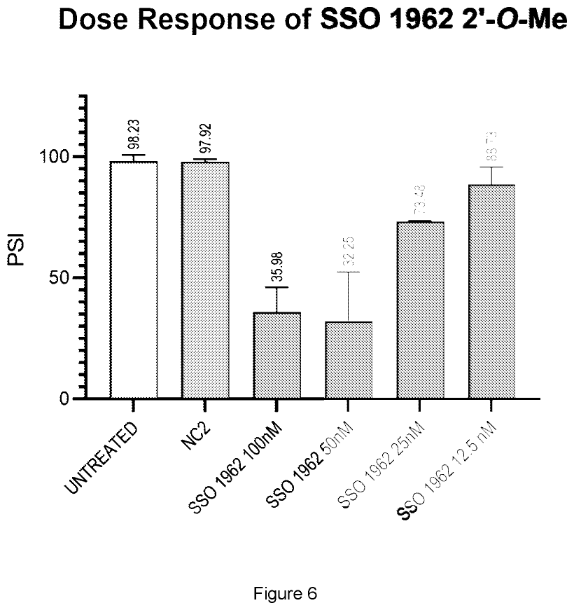

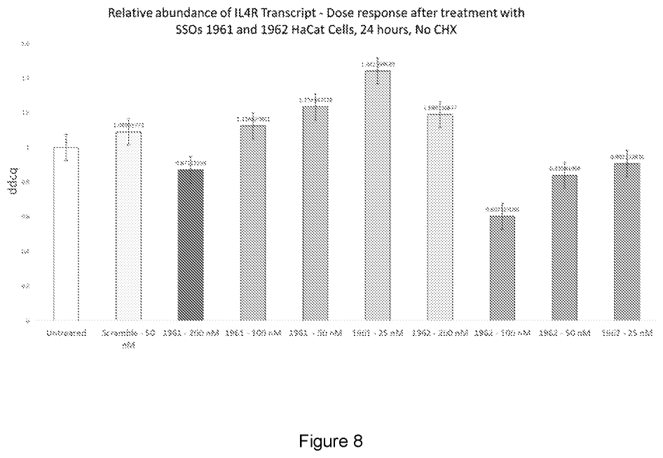

Downregulation of total IL-4Rα transcripts by each of the 8 SSOs was investigated along with Scramble SSO as negative control. Out of the 8 SSOs only SSOs 1961 and 1962 were observed to reduce the abundance of total IL-4Rα transcripts (FIG. 5). Although demonstrated to be efficacious in inducing targeted exon skipping, all but SSOs 1691 and 1692 failed to reduce the abundance of total IL-4Rα transcripts, suggesting that NMD is most active on resultant transcripts without exon 8. Of note, exon 8 which is the target of SSOs 1961 and 1962 encode encodes the transmembrane domain while exons 4, 5, 6 and 7 which are the targets of SSOs 1571, 1790, 1572, 1791, 1792 and 1793 encode the extracellular domain of IL-4Rα protein. From the dose responses of SSOs 1961 and 1962 in downregulation of total IL-4Rα transcripts (FIG. 8), SSO 1962 was observed to be the more efficient lead in downregulating total IL-4Rα transcripts. Subsequently, the dose responses of SSO 1962 in inducing exon 8 skipping and downregulation of total IL-4Rα transcripts were determined (FIG. 6).

### Example 6

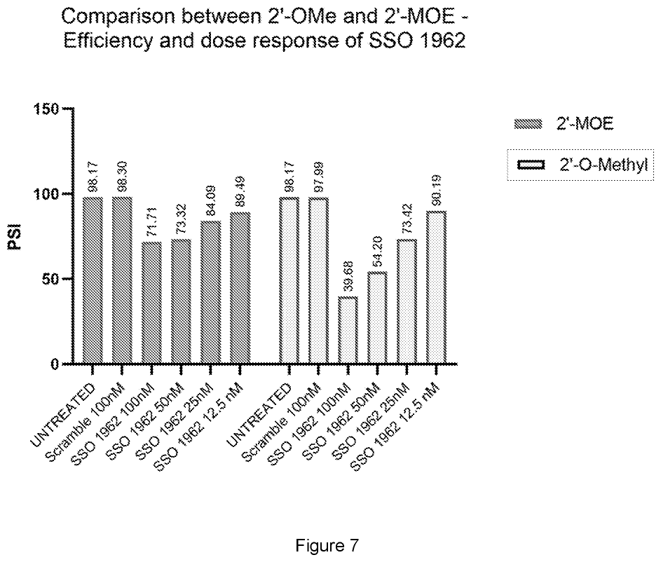

The efficiency of SSO 1962 in inducing target exon skipping when fully modified with 2′-O-methyl (2′-OMe) was compared with SSO 1962 when fully modified with 2′-O-methoxyethyl (2′-MOE). As shown in FIG. 7, 2′-OMe-modified SSO 1962 is more efficient than the 2′-MOE-modified counterpart at 50 nM concentration onwards.

### Example 7

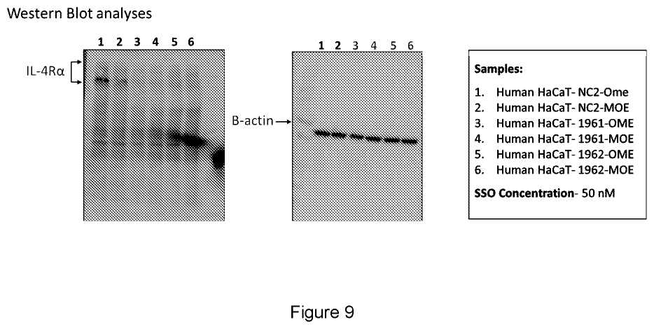

Western blot was performed to measure the efficacy of SSOs 1961 and 1962 in downregulating IL-4Rα at protein level. Notably, at 50 nM SSO concentration, the extent of downregulation of IL-4Rα protein abundance (FIG. 9) is significantly greater than at the corresponding mRNA transcript abundance (FIG. 8). The extent of IL-4Rα protein knockdown is similar among 2′-OMe and 2′-MOE modified SSOs. In fact, the protein knockdown efficiency corroborates with the efficiency in inducing exon 8 skipping (FIG. 4D, FIG. 6 and FIG. 7) more than mRNA knockdown efficiency (FIG. 8), which suggests that NMD is not degrading all resultant mRNA transcripts efficiently.

### Example 8

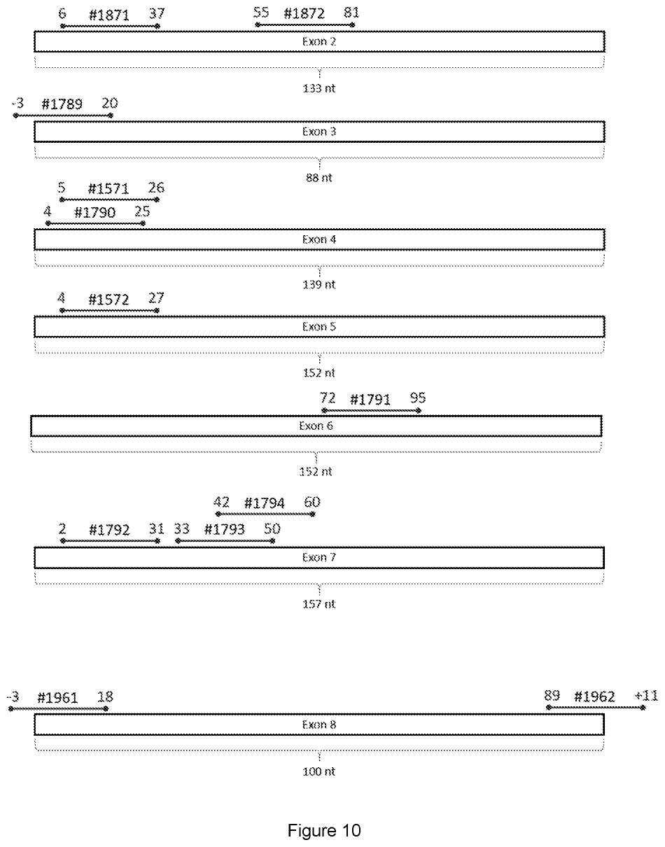

Current state of art in identifying effective SSOs uses a semi-empirical approach where hundreds to thousands of SSOs, each walking around and along the target exon are synthesized and screened, which requires considerable resources and trial-and-error. Not all antisense oligonucleotides can function to induce exon-skipping. For example, Table 4 lists the sequences of SSOs that are effective in inducing exon-skipping. Table 5 lists examples of SSOs that are not effective in inducing exon-skipping. Only specific positions or segments within the pre-mRNA sequence can mediate exon skipping when targeted by the SSO (FIG. 10)

In various embodiments, every sugar moiety in a SSO is modified with 2′-O-methyl (2′-OMe) or 2′-O-methoxyethyl (2′-MOE).

TABLE 4
Sequences of effective SSOs

| SEQ | | |
|---|---|---|
| ID | | |
| NO | SSO | Sequence (5′ to 3′) |
| 1 | 1571 | GGG CUC CUG CAA GAC CUU CAU G |
| 2 | 1572 | UUG UUC UCA GGG AUA CAC GUG UGG |
| 3 | 1790 | GGC UCC UGC AAG ACC UUC AUG U |
| 4 | 1791 | GUA AUU GUC AGG GGG AUA CGG GUU |
| 5 | 1792 | GUU CUA GGU AGG UCA CGU UAU AGA |
| | | UUC UGA |
| 6 | 1793 | GCU GCG AUG CGG AGG GAG |
| 7 | 1961 | CGA AGG GCU CCC UGU AGG CUG |
| 8 | 1962 | CCA GGA CUC ACU UGG UGA UGC UG |

TABLE 5
Sequences of non-effective SSOs

| SEQ | | |
|---|---|---|
| ID | | |
| NO | SSO | Sequence (5′ to 3′) |
| 9 | 1789 | AUU GGG AGA UGC CAA GGC ACC |
| | | UG |
| 10 | 1794 | CAG GGU GCU GGC UGC GAU G |
| 11 | 1871 | UAUGAUUUCUUCCAGCUGUGUGUAAAU |
| | | CUUUA |
| 12 | 1872 | AAAGUGCUGGGAUUAUAGGCAUGAGCC |

### Example 9

Table 6 lists the sequences of exons 2 to 8 of IL-4Rα, including parts of the introns flanking each exon (50 nucleotides of intronic sequences at the start and end of each exon).

TABLE 6
Sequences of exons of IL-4Ra

| SEQ | Description of | |
|---|---|---|
| ID NO | sequence | Sequence |
| 31 | IL-4Rα exon 2, with | gggaattggctcttttcttcattttcttaaattaaactttttattttcagataattaaagatt |
| | the first 50 nt and last | tacacacagctggaagaaatcatagagaagccgggcgtggtggctcatgcctataatcccagc |
| | 50 nt being intronic | acttttggaggctgaggcgggcagatcacttgagatcaggagttcgagaccagcctggt |
| | sequences flanking | cagtatgatgaaaccccatctctactaaaaatacaaaaattagttggg |
| | exon 2 | |
| 32 | IL-4Rα exon 3, with | ctctgctggaagcacaggtcagcactaacctgcttcctctctctctgcaggtgccttggcat |
| | the first 50 nt and last | ctcccaatggggggctttgctctgggctcctgttccctgtgagctgcctggtcctgctgcag |
| | 50 nt being intronic | gtggcaagctctggtaagtcaccacttctcaatcattcatttgttggctattaatggcgtgc |
| | sequences flanking | c |
| | exon 3 | |
| 33 | IL-4Rα exon 4, with | ctcacgcattgagttcctgggccgctcaggctgctcctgtgtctccccagggaacatgaag |
| | the first 50 nt and last | gtcttgcaggagcccacctgcgtctccgactacatgagcatctctacttgcgagtggaaga |
| | 50 nt being intronic | tgaatggtcccaccaattgcagcaccgagctccgcctgttgtaccagctggtttttctgct |
| | sequences flanking | ctccgagtaagcctgcgctggagctggaggtttggggaggttgtgcccaaagggtt |
| | exon 4 | |
| 34 | IL-4Rα exon 5, with | gcccagccagcctacaggtgaccagcctaacccagcccctgtgtctgcagagcccaca |
| | the first 50 nt and last | cgtgtatccctgagaacaacggaggcgcggggtgcgtgtgccacctgctcatggatgac |
| | 50 nt being intronic | gtggtcagtgcggataactatacactggacctgtgggctgggcagcagctgctgtggaa |
| | sequences flanking | gggctccttcaagcccagcgagcatggtgagcagggcggagtgcggcaggggtggct |
| | exon 5 | gggtgtgttcccacagctg |
| 35 | IL-4Rα exon 6, with | atgggggagtcactgcatagatcctcacatagaggccgcttctcccgcagtgaaaccca |
| | the first 50 nt and last | gggccccaggaaacctgacagttcacaccaatgtctccgacactctgctgctgacctgg |
| | 50 nt being intronic | agcaacccgtatccccctgacaattacctgtataatcatctcacctatgcagtcaacat |
| | sequences flanking | ttggagtgaaaacgacccggcagatgtgagtgggcatgctttgacgtttttctgtgacc |
| | exon 6 | tctggggaacagggtg |
| 36 | IL-4Rα exon 7, with | gtgcccctcgcccccggctggtgccctaacatctcccttttctctaccagttcagaatc |
| | the first 50 nt and last | tataacgtgacctacctagaaccctccctccgcatcgcagccagcaccctgaagtctgg |
| | 50 nt being intronic | gatttcctacagggcacgggtgagggcctgggctcagtgctataacaccacctggagtg |
| | sequences flanking | agtggagccccagcaccaagtggcacaactgtgagtatcaagaggcctaagcaatggta |
| | exon 7 | atctccactctccattcttcc |
| 37 | IL-4Rα exon 8, with | aggctgtaccatggctgacctcagctcatggcttcccctcccacttccagcctacaggga |
| | the first 50 nt and last | gcccttcgagcagcacctcctgctgggcgtcagcgtttcctgcattgtcatcctggccgt |
| | 50 nt being intronic | ctgcctgttgtgctatgtcagcatcaccaagtgagtcctgggcccagtgctgccgagca |
| | sequences flanking | gtccctctggagtgcagggtg |
| | exon 8 | |

The DNA sequence of IL-4Rα (Genbank Gene ID: 3566; Ensembl: ENSG00000077238) is as follows (SEQ ID NO: 38):

gcagagttctccgctgggcgtgacctcgggctacggcgtgggaggaagcgcgcggcaagacaccca
gcgaggtgctggggtcgcccccaggagaggacggcggctcggactgtccggcggcggcggcgggg
acagcgacaggggcgcgaggtggccgggacccgggccgggcgcgccgggcggggcggcgcatgca
aatctgccgggcgccggggggggagcaggaagccggggcgggctgggtctccgcgcccaggaaag
ccccgcgcggcgcgggccagggaagggccacccaggggtcccccacttcccgcttgggcgcccgg
acggcgaatggagcaggggcgcgcaggtaggatccggggcccgcgcgcggatcgggttgcgaagg
tatcgcccgggcacgccggctgagggcgttcgggaagggctcggccgccggcggggaccacgggg
accaccccgactccgagcggggcccgagcccgcgactctcggtgcgcgcggagcagcgcccggtt
ccgtccttgccgccgaacggcagcggaggcgcgaggcccggggtacgtggacacccagcgctccc
caaagccggtgctggcagtgagacctccgccgggacggcctgcgggggtggggggcttggggtta
ggctgtcggaggccacgcagcccctcttctccgggcagtggcgcccagccctgcgctcaggaagt
cagtgaggacttcggagagagaaagggtggggaaagttctgagaactgtaaatttgagtagtagg
tcagtgaatcggggcggtctccgcctccgaatatcagatggacccaataatcgcgatcattgact
gggacttgttttactgaaaggatgccaagtgaaacctcccactaacttctctgtggccggtgctg
tgcgtcatggacattgtacagatgaggtcaccaagaccccagccgctttagtaacttgccctaag
gtgccgcccacttgggagtccgtggggcagcaggactagagcccaggcaatcccacgccagagcc
agctgctctccatctctcaatcacgtttgaggagcccccacaataaccagaatctccgggagatt
tgcttaaaatgcagattcccaggtccttgccctgagattcagattcagtaaacccaggaatctga
tctttattttatttttaaaaatttattttataaagatggtgtctcactatgttgtccaggctggc
ctcaagtgatcctcccacctcggcctcccaaaatgctgggatgacaggtgtgagccactgagccc
tgattttttttttaaacaaaatctggatttcttagacttaagcaagataggggaccactagtttg
tctgcttctggggttcaaggaatgcccccaggccagcgctaaccctgctccagttctagtctctc
cccttccccagccctctgggaggcatgacccactgcacttccctgcccccagtagcctatgagga
gcccaaggcatttgctgggacctcagtgcctttatcaaaatcccaaagtccaatggggaaacagg
ctcaggtgtaggtttggccagggtgtcctgcacattagggcattccagggtcctgcaggggtttc
ttagaactcggctagcccagcccatgaatgggaggtgacctgctaagccactcattcactcacta
gcactcattcagctcctgcgaggccctgggtggtgttctgggcactggtgcttgcctcaaggagc
accccctctgcaggaagacagacgtgagggctgggagggtgcggatgcaccagcggctgcaggac
cacaagccagaggggatcagctcttcaaaggtcactgggccaggcttcccagcacccagggaccc
tgagctagccttgtgagtagagcaggggagtctggcaggtagaggatgggaggtaaagggaggca
cagagtgggcggaggctgggaagtgtgaaagggcactgtgtacagaacctagggagtaagcagaa
gccagagatggatggaaagttgggctgggctgcattgtggaaagccttgaatgccatgccaagga
acttgggccagtgtattcaccttggcttgagttgtaaaagcaacaatgcagattcctgggcccca
ccctgggcctctggatctgaatctggagggagacgctggggaggcagcagggaggaaaacctgca
gtcactcaccccatttacaacacatgcagttaccggcaggtccgagataacctaagggtttccaa
atgctaggaagtgactcaatgactgatatgtttaatacaatacaatcccggttgcaaaatgccca
gggcaaggggggggtgttcagttttctaatgctcccagttcaaaaaggagctgaaatgaattgtc
aaagaagtgacaagctgggcgcagtgactcacacctgtaatcccagcactttgggaggctgagag
aggatcgcttgaacccaggagtttgagaccagcctgggcaacatagtgagatcctgttcctacaa
aaaaaatttaaaaacttagccaggcgtggtagcaggtgcctctagccccagctatttgggaggct
gaggtgggaggatggcttgagcccaggtggtcaaggctgcagtgaactatgattgtgccactgca
ctgtagcctgggtgacagagcgagactctgtctcaaaataaacaaatacataaatacatgagaag
agagaaccttttctacctcccccatctccctgccctcttccacccaccccatctgggacgatgga
ggagagcgtttgggattagatgccccacctctcgactgccctcactggtacctgggagtgaggcc
aggacagaagccgcctaagtactgttcttcctcacatttgtaaaagtgaactttcttgagggttg
atgacatgttttataagctacttcacacaatgtccaacacccagtgggtccacaatcaatgtata
tgtctgttgttacctgtcaaaacgtatagctggggagtgacagtatggacttgagccagatcacc
cagtgcaaatgctggctttgctgcttactgtgtaactgacttcccagagcctcagtttctgcatt
tatgagattatagtgactatgccattgtagtgatgactaaatgagttaatatatgtcaagcatac
aaaacagtacctggtatgtagtacaaactatgtatttgttaaatgaatacctttttttttttttt
agagccatggtctctctctatttcccaggctggagcacagtggcacaatcatagctcactgtagc
atccaatacctgggttcaagtaatccttccacctcagcctcctgagtagctgggactacaggtgt
gtgccaccacggctggctaattgtttttatatttttagagacaggatcttgctatgttgcctagg
ctagtcttgaactgctggtctcaagcagtcctcctgcctcagcctcctaaagtgttggatttata
gatgcgagcaaacgtgcctggcctaaatgaataaatctttactgttgcctcctttgagctttatt
ggttgggatttagtccactcattttgcaggtgagaactgagacccggagatgaatgcagtagcct
tgcctcaggtccacatctgagggacagactccatcctacttttcttcccatgtattctttcctgc
ttgtaccagttcactggggtgaccaagaaagacatttctgagttttcctcctggctggcacagtg
gagacatgcccagccacgtttagctagacttaccatggctgggctagaagagaggccaggagctt
gtctggaggttgcacagacctgcccttggtgaatgtggatcggagcgccccggcagaggcttgag
gtgctacagaggctgggggatccactgtcagaccgggtgcatcaccactgttttatgatatccag
caaattgctctgcgtctctgagcttaaggcagagagaagggcctgagcttgaagaggggatccca
ggtttaaatcggagctggctacttcccagctctccactgtgtaatctggaggaagttgtttaacc
tctctgagcctccctttgttttgtcatgggtgtttgcagggagagcattcatgagatagtttgta
tagacattttgcccttggaaggcgctcagtgattgctgtttttctctgcattttggaccgagtcc
cttctgcgtatccagcacctggcttcctcctgcatttcttcctcaggaatttattgagcacctat
atgtgccagagcatcggaccaagtcctttgctctcctggggtttagattctagttgagcatacag
gcaatgtacaaccaatacatatgttcagatcggcagctttatctgttattctgaaatccacgatt
tctgaaaaccgaaagtttttcataactcattttgaagctaaacttgaggctttttagagtcttca
tcctcttcggtgtgatgctcagatgtctttctgcagagatattagtgcaattgatctttgggtgc
tttcccgccctgcatgatgggcatgtcggtgggtattttatacaaggtgacctttctacagcctg
agtcatcctgaattctgaactatgtctggcccccaggattttacaggagagaacatggacctgca
atatcctatcaggtggagactgtgctaggaaggaaagcaaagctgggtgcaagtggaatcaggga
gggcttccctgaggaggtgacatttgagagggatgaggaagagagggccaggtggagggactcac
agaggacggaagactccacttcctgactggcctgtgcttacccctcattctgcatgcacagttct
gtctaaatgcgtccgttcttgcctttccaagaagtgctgatgtctcttacgtttcaagtctagct
ctgactgactcattctctctttaggtgcccccacaggcctcctcacctcttctcgtttgcttaag
aaatctgaggtctgcgctaaagccagcctgggtgctgtaacctctcggccgaggatggtgcccag
ctgggcttgcatctggagttggaggaagtgggtcccgggaggggaagaggagccgtggggccttc
caagggtttggtggaattgagttcctggtggggaggggctgtgttctgggtcttagagcctctcg
cctgcaaagcaaagttgaacgtaacacgcgctcacatatctcgagtgccagcccggggctaggtg
cttaacgcttgcttagccaaagtgccaggggctggggacacgggaatgagtgacccagaaatgag
ggccctcatggagctgaaagcattctgggcttggtgactttgaggaagtggcttttgaactgaga
cctgaaataccaaggcatgtggatatctggggaaagagtgatcctggcagtgggaacagagagtg
caaaggcccagaggcatgaatgagcttggtgtgtttgacccacagcaaggaagctggtgtggttg
gatggaaccaaacaagaaagaggtctaggagcagaggtcagggagggagctggggccagatacct
gcattgtctcatttaacttgtgtaataaccctaccagataggagctactctcatgcaaatgttac
agatgaagtaacagaggcacagagaggttcagtgacttgtccagggtcacacacctgaaggtggc
agagcagggctgtgaacctagaagctccagagtcctcagtcctcaccatggtgtttaaacacaat
aaaatctatgcctgagtgctgaagacacacctgccatttagatcgatccacttttttacattgtc
ggtgtactccctgccatccatctgcccagaaatactgtgaatgtgggcagcgcaggactggttcc
tccaagccccattttacagacagtccaatcattttacatattattttatgaatgagcaccttcct
cagtgtctcagaccaggggtccccacacagcagggccacacagcaggaggtgagtggcgggtgag
tgagcgaagttttatctgtatttacagctgctccccatcgctcgcattaccacccaagctcggcc
acctgtcaggtcagctgaggcattagatgattctcacaggagcacaaacccttttgtgaactgca
catgtgagggatctgggttgcgtactttttcttttttttttaagacggagtttcgctcttgtcgc
ccagactggagtgcaatggtgcgatcttggctcactgcaatctccacctccccatgttcaagtga
ttctcctgcctcagcctggtggctgggattacaggcctgtgctatcacacccagctaatttttgt
atttttagtagagacagggtttcactgtgttggccaggctggtgtcgaactcctggcctcaggtg
atccacctgcctcagtctcccaaagtgctgggattacaggcatgagccactgcacgcggccctgg
gtgcgtgcttcttatgagactctaatgcctgatgatctgtcactgtctcccatcgcccttggatg
ggaccatctaattgcaggaaaataagctcagggctcccactgattctgcattatggtgagttgta
taattatttcattatatgttaccatataataataatagaaataaagtacgtaatacatgtaatgt
gcttgaatcatcccgaagccatggccccaaccacggcctctggcagtggaggctggagtccacat
cacgctctgtgctgggtgcaggaatggagatgagtgggtgtcctgctcatttccctgaggaggaa
cctgaggctcaggctagagacttacctgggtgacacggctcctaagtggcagagtccagattcaa
acccagggctacagacccagaagcctgtgcatttaaccacggttctgagctatccagcccaaaga
ggagagtttaaaaggattacatttcttcgttggaaatcagtgagcatagtcgcccagaaccattt
ttgaaaaactcagagaaggcctatgttttcctgaagcggccagggaagtattctgcctttttaaa
agggaagactgaattctagggaggagaggcaccagctctctgggctgggcaggtctccaagctgg
cagggccacagcctcccgacggtcttgcctctagcatatttcttccagaagccacatttccacag
gtgggcagctctaactgggagaaaatcttcctgagatttagccaaattagctttctttctttttt
ttttttttttgagatggagtctcactctgctgcccaggctggagtgcagtggcgcaatctcagct
cactgcaatctctgcctcccgggttcaagtgattctcctgcctcaacctcccgagtagctgggat
ttcaggcacacaccaccacgcctggctaatttttgtatttttattagcaatggggtttcaccatg
ctggccagactggtctcaaactcctgacctagggtgatccgcccacctcagcctcccaaagtgct
ggtattacaggtgtgagccaccgtgcccagcccaaattagctttctaggagcttccactttctga
gtctgtttctgcctcttggaggtagtgaccatctccctccaaactcggatacttctcttattcag
actacacaactgtagttatttcaggtattctccctgactcaggaaattataagtttggcctgcct
tggcacatgagtcaactggttggagtcccacgcaaagactgtttgtttggtcaaagaggaaggag
actattacctccttggatctagagcttatacttctgttgatgcaacctggaatcatactgtgtag
cagccatatcccacttggagtgcatattgggcttgtcagctaaaatctccatgtttctcttctat
atgagctgctgttaatccaagtttcttctattctgagagtccttgtctaaagctaagaggttcca
gtttttcatgctctctaataaatctacttcaggctaacatgctatcattttttctttgagtactg
ctttggtttctttccacaacttgttacacaatgttattcactgcccagtttagaatttccctttt
ggttcccattttaagacttatttaattatatgctttttaaatttttgagacagatagaatttttt
aagttatctttttattgttgatttctaaatttggtcatcatggttgttgtggtcaatatatgtgg
ttcctataatatcagtggtagagaatttgctgagatctttataggattttctggtcatttttttc
ctcttgtgtttgaaaagaatgtatattttctgttgggtataaggttctagaaatgtatctatttg
atcaagctagctattatattatctatatttgttatgtcactatgtggtttttttttttttacctg
atttacctcccaccgtaatacaattacaattcaccataatagaggctttgttaagttctcctttt
gagtctgtcagttttttgctttatttttgaagctatgttgttaagtgcatagccatatgagtaga
ataagggttcaggatttttatacctttttggtgaattgttgcattttcccgagtagctgggacta
cagatgtgcactaccacgcctagctgatgtttgtgttttttgtagagatgggatttcgctatgtt
tcccgggccagtctcaaactcctaggttcaagcagtctgcccacctcagcctcccaaagtgctgg
gattacaggcatgagccaccgcagctggctgaattgttcctttaatcagtataaaagagccaact
tccagcctgggcagcatagtgagaccccatctctaccaaaaactaaaaattagctgggtgtggtg
gcgtgcacctgtagttccagctacttgggagggcgaggtgggagaatcacttgagaccaggaatt
caaggccgcagtgagctatgattgctccactgcactccagtctaggtgacagagtgagacattct
caaaaaaacaaaaacaaaaaaaccatccaccttctctcttctaatacttactcaagacatcattg
tcatgaattctattttattgatgtcagtactcctgtactaataagccagatgagttaaaaacaac
agcagccttgcaaccagccatgaagcaagtgatcttattctcatttcacagatgaggagactgag
gctctgagtggactccagcctgccacggtctcccaccgagtcaggttggagcctgcacttaggcc
cagagttatgcttctgtgtctcagatgcttgggggtccctgcccacaaaggtggcccccatgtct
gtgtgtccccacccatccaggaatggtatggggtgagacaacaggaagagctccatgggtagtcc
ccacccactgccttccatatgccagtctgcaagcagaaaatgaacgggaagctcttacctgtgcc
catgtgttgtcctggctttgggtttgcccagggggcaaagtcctggtcattgttgagacaagttc
actgctgctgagggccgagcaaatggtgacaaaggccagattctaaattcctacctggttacttc
ctgctccgtcaccatcacctcgggcaaattctgaacctccctcagcctcattcctccatctgtgc
ttgcagggagaatattttactgtatcttccatagtgagctttccaggagaagcaaaaaagagtgg
tgtataaaaaagcaagtttatgactgcttctgtgccaggcactgtgctgtgcactccagagccaa
aggcctgggttcaaatcccagcccactcactagctgtgcatccttaggcaggttcctgaccctct
ctgggcctcggcttctccatctctgcaatggagatgatattaattgtatctgcaatgctccttgg
attgggtccatggttaagtgagtgagtgaggctcaagggcttctttttttttttttagagatagg
gtctcactctgttgcccaggctggagtgcagtggcatgatcacagttcactgcagctgcgacctc
gtgggctcaagccatcctcctgcctcatctcccttagtagctgggactacaggcatgtgccacca
tgcctggcaatttttttttgtatttttggtagagacggggtcttgccatgttacccaggctggtc
ttgaactcctggactcaagtgatctacctgcctcagcctcccaaatggctgtgattacaggtagg
agccaccgcgcccagccttcaagtgcttttttttgttagtgagactggtacaaccaatgccaagg
ctggtgagcaaagttgcacccagttggggaggggcgaaaccaagggcagaagggcccagggcatg
gcctctcatggctgaaggaagaggggacacgtggccagtcacagtgacctctgcagacctcaaga
aggaggccagctaggagccatagctgtcactaagacagtcaaaatctaatcagcaccctggcgcc
aagaggccattgtccgctgctcaggacagtgagtgatagtcgataaccagctctctggaggtcag
ggccccgaggtggctgagatctgtgcctgcattaggacatttaaggggactccaagccaggattt
gctagtgtttgcccagcagggagaggtggaaaattggggatgaccaaccaggcctccctgctcac
agccaggggaattcatcctgtcgttcttccattcagcaaatattcatggagcacctgctgtgtgc
caggcaccgtgctaggctctggggagatggctgtgagcgggatcgacactggccttgtgtgagct
ggccttgcagtaggggatatgagcaccaaacacataaggcccgagtgtgcaatatgccacaaaga
ttgcagagaaataaagcaagataacaggttgcagaaagatggaggccaggctggctgctgttcct
gggtaggtggcttctggacacagtttcctcaggctctgtttcctcctttgggcctctggaatctt
ctgaagacaggcctggcttccaggatcccggatggagttgcctgttgactgtgtggactcaaagt
gtccacagtgcgccggagcgcgtggtcttccccagcaaatctgcttctcttctcggctctccatc
gtgggaagggtccctgggatcctccccaccaccccagcctgaggccactttccctctgtccccgc
tctccccagcctgctttccccaccaagcgccaccaagtccctgctgcctcctcctcccaaacagg
ctcaaacctgccccctcttcgccagctctcagctcagcccgctcctctcctgcttaagagccttc
actggctccccactgccctagagacagagtttgagtctactgccatggcttggcagacggtgttg
tccccggcctgccaaccttccagctgccatcccagccagtccttctcccccttgctccagccaag
ggccccgcttgtagggtgggaagtgggccgtgccctccctcctgcctgcctattcgctgggtctt
ccctcattccctccctcaccccgccctctcataagaaccttcagaatctgccagacgcagtggct
cacgcctgtaatcccagcactttgggaggctgaggtgggtggatcacctgaggtcaggagtttga
gaccagcttggccaacatggcaaaaccctgtgtctgctaaaaatacaaaaaaattagctgggcgt
tgttgtgggcacctgtagtcccagctactcgggagactgaggcaggagaactacttgaacctggg
agacagaggttgcagtgaactgagatcccaccattgcactccagcctggactacacagtgagact
ctgtctcaaaaaaaaaaaaagaaccttcagaatcataaaatgggtttaataattatacttatctc
atagggagggcttaaagcacctaggccagtgtctggcaccaaggaaacactgtatacctgttaaa
ataagaataaaataaaatacacatgctcacttattgagtgcctgctgtatgtcgggcccctcccc
gagagaactctggtgcatccccgataaagaaacctgaccctctaggatgagggagggtcttccct
gagcgcctgctaacagcctcctctgctaatctgaagtcctgcagcttgcccagcaggctggcttg
tcacacgggctacccccacaggccgccatcttggaaaaggccaatggctgtggaatcattcttga
ctcctattagccttcctgttctctctcccgccccacagcatccatcctgtcagcaaatcctgtgg
gctctgtcttcaacacagatctagaattcagccccttctcaacctccccatctccgcagcctggt
gtgagccctggcacctctctctggggccagtgtggtagcctcctcactgctacccacttctaccc
gtgcccccaccacccaagcaagagaggtcttttttaaagtctcatcatgtcacttctttgctcaa
aacgctctggcaacttcttccttgctcaaatatttgttaaaagaagaaaggaatgaatgaatgaa
tggcctgtttgcatctgtaatagccagcacataggcagtacttggaaaacacccacagcaagaat
aaatgaggagatggctggacacggcagctcatgcctgtagcatgttggaaggccgaggcaggagg
tttgcttgaggccaggagtttgaggttgcagtgagctatgattgtgccatagcactccagcttgg
gccacagagagagatccccatctctacaaaaatttaaaaattattcgagcatggtggtgtgtgcc
tgtagtcacaactacttgggaggctaaggcaggaggatcacttgagcccaggagttcaaggctgc
actgagccgtgacggtgcgactgcattctagccacagaatgagaccctgtctctaaagcaaaaaa
gagaaggagaataagtgaggtacttgagcaagtgggtgaggatggttgagccacagcaggcccac
agggaggctggtgggtgctgttggtggagggggccgatttgtgttcctggtggctgcagagccag
tctgttgcctcccctccctcccactggctgggctggggacggagccaaccaggcagatcccgggc
cattctggggccctggcctggcctgccttactatcccagtggggccaccgacgtggctgccattt
ctggaaaccccagttctaggtaggttactgtcccaggaagaagtgcatgcctagcttcctgccag
cccagcccagctgtcccagaagccagaggaagctctgggctggtttcttataagtagcatgacta
actgattgcgccacaggagctgaggtttcaggccagtttctgtcgtggagtttcaggggtcagcg
gctccaaagccccagcccccacccgaggcccctgctgtccctggattcctctccacatgccacct
gtcacagaggtgcttgcatcctgtctgcagaaccagcccctcctccaagccaggtttcctgccca
gagggaagaagagcagaaggcctgtaccgccccctgtgcgcccccagtcctccgccccctgcccc
cctgccacccgctgtgggttcgggaagagtcctgctgggtcgcttccaggctgctgctggcttct
gacatttcaaggcttaatcacttaatctcccccgagttttttaaaaaaagtgagtcaagtgtgtt
ctcctggtggcgggcgtgttttcccaggcccatgcttctgctcatttcctggctgcggccttcac
tgactttgctgcttcctaactctgtgaccttggccttgtcacttgctgtctctgggtctcaggcc
aatgggtctcaggccagtggggctccccggacaaagttccagggttttggggactgcatttcaag
ggtcagcaaccacagtccttaaacacgtggattttggagtgacaatgcctaggttatggttctgt
gaccaccagcaagtgctctagcctttttgtgcctcagtcttctcatcagtaaaatgggaatgtta
atggcacctacctcatagggtcgttgggagaaccaaattgattaatatattgtaagtacttagaa
cagggccaggcacaaaataagtactgcatatacagttaactatggacaaaagcttgggacctggt
tcatgtttaaaaatgattattgaggccaggtgcggtagctcatgcctgtaatcccagcactatgg
gaggctgaggcaggcggatcaggaggtcaagagatcgagaccatcctggccaacatggtgaaatc
ccgtttctactaaaaatacaaaaattagccaggtgtgatggcgtgctcctgtagtcccagctact
caggaggctgaggcaggagaatcacttgaacccacgcggaggttgcagtgagctgatatcgtgcc
actgcactccagactggtgacaaagctaggctccatctcaaaaaaaaaaaaaaaaaagattgttg
aatttattattgctgaatttttttttttttgagacagaactttgctctattgcccgggctggagt
gcaatggtgtgatctcaactcattgcagcctctgcctcttgggttcaagcgattctcctgcctca
gcctctcagcctcccgagtagctgggaatacaggcacccaccatcatgcccggctaatttttgta
tttttgtagagatgtggtttcaccatgttggtgaactcctgacctcaggtgatccacctgcctcg
gcctcccaaagtgctgggattacaggcgtgagccactgtgcccagcctattattgctgactttta
aaccattcattcatttaatatatgtttattgtacacatgctatgcctgtgtcaggcactgtttca
ggcactggggataaagcagtgagcacagcagacataaagctctgccctcatggggcttgtggtct
ctcagagggaggcaaacaatgcatttatagacaaatacataatttaaatttgaggttacctatta
tactcccaacttgaaaaactagcagtatattgaggtggctatttcacatctgatggaggaatatg
tcttttgtgaaccatttatgttctttgcctatttttcttaaagggatgtttgtcttattgatttg
taagaactcttcatatattaaggccattaaccgttcataatgtagattgctacctttttctagtc
ttttgcttgctgatatagttgggatgtcatcctctctaaatctcttgttgaaatgtgatccccag
tgttggaggtgggtcctggtgggagatgttgggtcatggggacgggtccctcatggtttggttct
gtccttgccatagtgagtgagttctcacaagatctggttgtttagaagtgtgcggcacctccctc
ctctctcttgccctggctgtcaccatgtacgcctgctcccctcttgccttccccgtgaataaaag
ctccctgaggcctccccagaagccaagtagatacgggtgctatgcttcctgtacagcctgcagag
ccataagccaattaaacctcttttctttgtaaattactcagtctcagatatttctttatagcaat
gcaaaaatggactaacacacttgagttctgtctttttcttatggcatattttaatctaggccaga
cacagtggctcatgcctgtaatcccagcactttggaggccaaggcgggtggatcacttgaggtca
ggagtttaagaccagcctggccaacatggggaaaccctgtctctactaaaaacacaaaaaattag
ttgggtgtggtggcacacacctgtaattccagctactcgagaggctgagacacaagaatcgcttg
aacccgggaggtggaggttgcagtgagccgacatcacgccactgctctccagcttggggaacaga
gcaaacctccgtctcaaaaaaaaaaaaaaaaatctagggaagttgaaaacattttgcagtcaaag
ctatctttgctatctgcatcttgaaataaaataaacattcatacatgtaacctcattaatctgat
tagagatagtgataagtactgggacagacataaacagggtgctgtgataagaagtcacagggagg
gcccgcattagttgaggggggtcagagaaggcttctgaagtgtttctggtgcaaatgcttggaaa
agggagagctgggagtgtatcccagggctgaggccaatggaggattgcagaagagggagagggga
taggaggggaatgttagagggataagcaggggaattggctcttttcttcattttcttaaattaaa
ctttttattttcagataattaaagatttacacacagctggaagaaatcatagagaagccgggcgt
ggtggctcatgcctataatcccagcacttttggaggctgaggcgggcagatcacttgagatcagg
agttcgagaccagcctggtcagtatgatgaaaccccatctctactaaaaatacaaaaattagttg
ggcatggtggcttgcctgtagtcccagctactggggaggctgaggcaggagaatcacttgagcct
aggaggtagaggttgcagtgagccgagatcacaccactgcactccagcctgggcaacagagtgag
actccatctcaaaaaaaaaaaaaagaaaagaaaaagaaaaagaaatcacagagagacccctgctt
ctatgctgttccccccgagggcaagattgtagaaatctgtaatacagtatcacagctgggatact
gacactgatataagctactgaccttattgggatttcccgatttacttgtactcatttttgtgttt
gcgtacgcatgtgtggtttagtgctatgcaattttatcacggcataggtttgtgtatgcagccag
gatccagcacagttctatcatcacaaggatggctcaaattctccttttatagccacatccaactc
ccttcctgacccaccccctcagcccctggcacccactgatgtgttttccatttccatggttatgt
gatttcaagtatgttacataaatggagtcacggggtgtgtactctgggaactggcttttttcact
ctagggcttggattttattctaagtcagagatgcttctctaatgagctgtgaaccccctggggat
ctgtggcccctgtgaagaagccctttaggagccagggggcattggatgtgtgagctggccctggt
ctcatttctgattttaaaatggctctggctgtgatgtggagaagaggcagggtgagcctcatctg
cagcaactgctaactgcccacatccccccagacttctcaaaaatttgccactcccctgctcaaag
accctcaaaagctgcccactgcttagaggacaggcctttgacctgaggtcctcacttgggcttca
gggattctgtgaacccctaaaatggaatgcagaattaagtgtatgcaccttgccagaaacagggt
ttggggtttacattagatgctcagataggtcattgatccccaaaacactaaggtctctgacttct
agacagatagtagttaagaaccaaagctttggagcttgagtgctggatttaagacccatgactgt
cacagctgtgcaacctcagacaaatcactcagcctttctgtgccttggtttcctcatctgaaaat
ggagaaaataatattacctatttcatagattattaaatgcattcttgtatgtaaaagatgtaaaa
gaaaacttggcatgtggtaaactctgtgtgtttaagtgaatggtgaggctgcccagtataggagg
gcagagctttccaggtaaagactttctaggcctcctgggattatcaggccctctctgtgttggtt
gttgtcatacatgacagcagtattccttccaaagggatgccaagttgtggttgcacactgcactt
agatccagttgattgacagcactgttcaagtcaactgtgtccttactgatttctgcctgctggat
ctgtcgattaccaaaagagttgaagtaatagtaataatactattattatccaataatagtattat
tcagaataatcacacagatttctctatttctccttgctgctctcagtttttgccttacatatttt
gaccctttgttgttagttgcctacacattaaggattgttatttacatcttcttggagaattaact
cccgaatcattatatagtgccacttttttttaatttaaaaattttattttttattttttgagaca
ggatctttctctgttgcccaggctggagttcagtggcatgaacacagcccactgcagccttaacc
tcctgggctcaagtgatcctcctgcctcagccttcccagtagctggggctacaggcatgtgccac
ctctaataacttttctttaatttacaatagtattagtttgttctcatgctgctctaaggacatgc
ccaagactgggtaatttataaaggaaagaggtttgactcacagttctgcagggctggggaggctt
caggaaacatattatcatggcggaaagggaagcaaagacatcctttttcacatgacgtcaggaag
gagacacgccaaggaaagggggggaaagccctttataaaaccatcagatctcaggagaactcact
cactgtcacgaaaacagcatgggggaactgctcccgtaatctaatcacctcccatgaggtccttc
ccccaacacgtggggattacaattcatattacaattcaaggtgagatttgggtggggacacagag
ccaggccatatcacattattattaattagtgccttctctctcatttttttgattcgcctaggtag
agatttatcaatttcattgatcttttcgaagaaccaatttttgatttcattgattttcctcgatt
aattttctatttttaatttcattgatgtatgctccaattttgtatttttttcttctgcttgtttt
agacatatattgcttttctgtttctagtactctagatggaaacagattattgattttagaacttt
ctttctaagatatacattcaataatataaatttccctctaagcactgcttttgctacgtctcacc
gattttgataggctatattttcatttttatttagttaaaaatatttttagtttctcttgtgattt
cttctttgatccatatgttattcggaagtgtgtttttaaatctccaaatacttggaaattttctg
gctttcttcctgctagtgacttctaatttgaatccattgtggcctaagagcatatgttgtgtgat
ttctaatgttttcaatctcttaaggttgttttatgtcccagaatgtggtctgtcttggtgattat
tccacatgaacctgaggagaatgtatattctgctgttgtgagatgtaacagtctataaatggata
cctctgttaatatgacctcgggtgactctttttttttttttaagttgctgcttcaccattggctt
ttggagattttgcagatgataaaatcctcaggacttttctgctgctcagtcagattttcttatcc
tacacttgcggaactgttgtttgaaattgacaagcaggatggccaactctgcccggctcatttta
tttcatcttattgggttcagcctgcagttttaggagatgagattgaaggaactggtatactggaa
tccacggtgggctcggaagcaaacaaacgtgggttctgattctagctctgaccttttaccagctg
tgggacctggtggatgggttatttggcctctctggcctcagtttccttagctagacagttgggat
aacagcctacctttcaggattcttgtgtggattcggtgaggtaatacttctaaagcacctagcaa
agtgtcaagtacaaggtaagtgctgggcaagcagggattttgttcacacaaagccttgactcaac
ataaggaggctgtttccaatggctctgcttaactgagacctgagagtgattgctccatctgtggt
ggcatatgggctgaaactgcctagcagcatgtcagaagtggggtgttcgcaggacatccttgtcc
aggttggcagttgagcttggcaggtgaccgctgagaccccttccaactccaccacttcaggaatc
tcttttttttttttctgagacagtcttgctctgtcacccaggctggagtgcagtggctcaatctt
ggctcactgcaagctccgcctcccgggttcaagccattctcctgcctcagcctcctgagtagctg
ggactacaggcacccgccaccacgcccagctaattttttgtatttttagtagagactgagtttca
ccatgttagccaggatggtctcgatctcctgacctcatgatctgcctgcctcagcctcccgaagt
gctgggattacaggcgtgagccaccgcgcccagccaggagtctcttgatttagaatgggaacttg
aaatcatctcctcttgccgtgtacttgaacgcgataacctggcacttatctttttaattctttta
ctcacctgcatctttgttctttttgcctctgatgttcagggaagcctcttctcccactgcatctg
tgatctctcctgaaatgtagcacccacacatccctgagaagctagctgcggcacgtgtgtgtgca
catgggcatgcgtaagtctgggcgtgccttccacaacagattaaggatatcaatcctgacgccat
gctgggatgttttagtgaatcttgacagggttgttgactcccctgtttccctggaggactttacc
atgaaagtcttctgctgtgtgactttggacaggcgtcttggcctctctgtttctctttattcata
attgaggctaaagtgacacctttgccatctctgccacaggatactgtgggcatctgtgtagttgt
gagagtaaaagttactaggacttgccaaggaaatcagtgtcaggaacccaccaccccattcccgc
ccctcctttacttgagactttacttttgtaactctcagcaaggaagacgagttcaacctttgagc
cgatacagagccttcccctagagagagctttgcagcctcatgctgagccagttaggggccccaca
tcatgtccaaaatgaatttaatcaacaaacagttactgaacacgtcctatgtgccaggcaccgtt
gtagatactggggactcggcagtgaatgggacagaccctggctgtcaggatgacagacaatttaa
aaagggacagatgtcccacgctggcttgtcctttgcacccctggcacttcgtgaggagcagcggc
cagttcttgtatatagactggcccttggtgtacatttgtctgacgtttccaaatgatgtatgcat
ccttggcaggaacccaggaaaagacatgctgtgctcttctcaggaattgtttcagggaggtgatg
ttggggatttgtcccattcttgttggtgagtttagatcacttggtgaaggtggtgtctgccaggt
ttctgtattgtaacgttcattgatatgtattttcactcattagttaatatacaatacaagttttg
cagggagttactttgaggctgtcatagtaaaatagagctttcccttcttccctgtttatttattt
attagagatgaagtctgactctgttgcccaggctggagtgcggtggtgcgatcatagctcactgc
agcctccaactcccaggctcaagccatcctcctgcctcagcctcccaagaagttgggattacagg
cacacaccacactgcacccggcctcctccatttattgatctatgtcaatacagactcctggactt
taactccaagggttacaagtggataccataatattcattttgatgctcaaaatgtgcctgttttg
tccaggtcaggctagctgctgagtacttccttattgtctggcacaagaattccaagcctatctgg
tacttttccttctcctggaatcagctatttctctaaggcgctctggttcctttcagtacagaata
gtatttagaagccaagacctggtcactaggaaggcatgataatcaaatgtgatgaatgatcttgg
attgcatcctggacgtataaagggcattattgaggcaattagcaaaacataaataggggccgtgg
atgagatagtggtgatgcatcactgttaatttcctgatttcgatggttgtattgtgattatgcag
agaatgtccttgtttttctgacatgcacactgaggtattggggtaatttattcattatggagata
tgtgagtgtgtttatgtgtctcatacacagatgtttcttaatgatggggttttgtccccataaac
tcatcataagttgaaaatattattaagtcagaaatgtacttcatactcctaacctactgaacgtt
atagcttaacttagctcggaacacttgcattagcctacagttgggcaaagtcatctaacacaaag
cctgttttgttataaagtgttgaatagcttgtgtagtttatggaatactgagagtgaaaaacaga
atggtcgtatgggtgctccaagtactgcttctactgaacacccattgattttgcaccatcataaa
ttaggggaagagaggaaaagcacgcgtaagggaggaagggccgggtacagtggctcacgcctgta
atcccagcactttgggagaccaaggtgggcagatcgcttgaactcaggagttcgagaccagcctg
ggcaacataatgagaccccatctctatcaaaaatacaaacacttagccaggcctcatggtgcaca
tctgtggtcccagctactcaggaggctgaggtgggaagattgcttgagcccgggaggtggaggtt
gcagggagtcgaggttgtgccactacactctagcttgggtaacagagtaaactctgtctcaaaaa
aaaaaaaaaaaaaaaggaaagggaggaagggaaggtccctccaggtggaggggacagagagagaa
aaggcatggaggtgggactgagtttggtgtgtttaagaagtgcaggtggcagccgggctcagtgg
ctcacgcctgtaatcccagcactttgggaggctgaggcaggcggatcacctggggtcgggagttc
aagaccagcctgaccagcatggagaaaccctgtctctactaaaaatacaaaattagttgggtgtg
tggcccatgcctgtaatcccagctactcagaaggctgaggcaggagaatcgcttgaacctgggag
gcggaggttgcagtgagccgagatcgcgccattgcactccagcctgggcaacaagagtgaaactc
tgtctcaaaaaaataaaatgaaataaaataaaataaaatgttaaaaaacaaaaaagaagaagtgt
aggtggcaggcgtaggatgcatgggtgctgggcccaggtggagagggaggtggagccctgctggg
gggagggccttcaagtgtgcccaagtcactcggagagcatccagcaggggcctagagctcctgag
gtggccttaagttcttatgatgccccagacactgcacacagtgcttcagaggcaacacctccgtt
cctgtttgctgcaagcctgtgaggttagccttggaaccattcctgtgccacagaaacacagccag
agcaccaaaagagaaatgctgttaatggcaaagaactactcactagcagaacaaagcctgggagc
tgcttttttgaataagatcccaggagagagacccaagcgggaagtgaaggatgggcacttgaaat
gttgcagggaatataacccagcgcgtgctgttttcttttttttttttttttttttttttttctct
ttttgcaacagggtctcactgtgtcgcccagcctagagtgcagtggtgccatctcggctcaccac
aacctccacctcctaggctcaagcgattttcctgcctcagcctcctgagtagctgggactacagg
cacgcgccactaccgcccggctaatttttttttgtatttttggtagagacaggtttcaccatgtt
ggccaggctggttgtgaactcctaaagtgctgggattacagatgtgagccaccacgcccggcccc
agtacacactttttggagggtatttgacgttatgtaccaggagtgctggggaaaagtacacactt
gactccgtgagattctgcttcttagcatttttttcttttcctttttttttttttttgagacagag
tctcactctgtcacccaggctggagtgcaatggcatggtctcggctcgctgcaacctccgcctcc
tgggttcaagcaattctcccacctcggcctcccgtgtagctgggactacaggtgtgcgccaccac
acttggctaatttttgtatttttagtagagacagggtttcactatgttggccaggctggtctcaa
actcctgacctcgtgatccgcccacctcagcctcccaaactttctgttttttttcttttcttttt
tttttatgacagggtcttgctggagtgcagtggtacggtcttggcttactgcagccttgacatcc
caggctcaaacgatcttcccacctcaacctcccaagtagctaggattacaggcatgcaccaccgt
gcctggctgattttgtttaatttttgtagagataaggtctcactctgttgcccaggctggtcttg
aactcctgggctcaagccatcctccctccttagcctcccaaagtgctgggattacaggtgtgagc
caccacacctggcttctgaacatttttttcttaatgataatagctggtgctatgggctgaatgtg
ttcccccaaattcatatgttgaaacttcatcccattgtagtcatattaagagatggggcctttgg
agaagtgattaagtcatgagggctctgtggttggtcatgaatggggttagtgccctcatgaacgg
gctgaaggaagtgtgtttgccccttctgtcatatgaagacacagcagcatggcatcgtccgtgag
gaatgggctctcaccagatctggatctcctagtgtcgtgatcttggacttcccagtctccagaac
tatgcattagaaaatttatttatttattatttatttattttgagatggagtcttgctcttgtcgc
ccaggctggagtacaatgacgcgatctcgactcactgcaacctctgcctaccccagttcaagcga
ttctcctgcctcagcctccagagtagctggaattacaggtgcccgccaccacactcggctagttt
ttgtatttttagtacagacggggtttctccatgttggccaggctgatctcgaactcctgacctca
ggtgatccacccaccttggctccccaaagtgctggaaatacaggtgtgagctactgtgtctggcc
tgaataataaaatttaaaacaatttttcaaaaattcaccatgaggtctcactatattccctaggc
tggtctcaaacccctggactccaagtgatccaccccaccttcccgagtagctgggactagagatg
cacaccattgcacccaatagagcaatacgtttctgttctttgtaaattacctgctctaaggtatt
tttgttatagcagcctatatggactaagctgacttgtaacgttacttgagactttaaagtgttcc
ggtcactgttggagggctctgtctgtgttagctcatttaatccccacaacacctcaatcagatgg
ggctattcttagtcccactttatagataaggaaactgaggcatggaagcacagcttgctcaaggt
tcacatctagtcagtgacagagcaggtatttaaacctcaggaaataatcagagaaacatgtgtag
agggttgtccaaggaaggccacatccagaagcatctcccaggacagttgttgtgtagctcaccct
ctggactttgtgggtctgggtgttgtttcatgattatagagagagctctgtgaacgtggaggacc
tgttgtcggcagagacacaaatggccagggcatggctgggcagccgcagtggctcaggcctgtaa
tcccagcacttcgagaagaccagaggggcagatcatgaggtcagaagttcaagaccagcctggcc
aacatggtgaaaccccgtctctactaaaaatacaaaaattagccaggtgtggtggtgggcacctg
taatcccagctactcgggaggctgaggcagaagaatcgcttgaacccgggaggtggaggttgcag
tgagctgagattgcaccactgcactccagccttggagacagagcgagactctgtctcggaaaaac
aaacaaacaagcaaacaaacaaacaaataaatggccagggcaggggagggttgcatattgaataa
gatgagctctgctggaagcacaggtcagcactaacctgcttcctctctctctgcaggtgccttgg
catctcccaatggggggctttgctctgggctcctgttccctgtgagctgcctggtcctgctgcag
gtggcaagctctggtaagtcaccacttctcaatcattcatttgttggctattaatggcgtgccag
ggtcctgcagtatgtcacctggccttatggagattacactgcagtgggaggggacagccaatgac
aagtggccctgattatcagtaaattctaaagattgttagaaagtgatgggagccgggtgcagtgg
ctcacacctgtaatcccagcacttcaggaggccgaggcaggaggatcgcttgagcccaggagttc
gaggtcagcttgggcaacatagggagaccttgtctctacaaataataaaatattagccaggtgtg
gcagtgcacgcctgtagccccagctactcaggaggccgaggtgggaggatcccttgaactcagga
ggtcaaggctgcagtgaactgtgatcgcgccactccactccagcctgcgtgagaaagtgagaccc
tgtcaaaaaaaaagagaaggtgatggggaaagaacacagaacagcataagagggggttggggaag
ctgggtggagtgggggggattgcagttgaaagtagggaagtcagggaaggcctcattgagctgac
ttggaggaagcgggaaccgtgcagatgtctggggaaggctcattcttggcagagaggccctgcac
tgagcctggcgggagggttgagcacaggagggaatgtggtggaggagagtgagcagcaggaggga
gcagtgaaggtcagcaaggtgacagagtggctgaatcaaaaaagaccttgcagtgtttgagcaga
ggatccatatcatccattatgttccaaaggactcttcaggatgccgtgtggagaaaggaagaggg
tggaagccaggaggtctggagggaggtctggagtggaggagatgagaggctccggatccctctgg
gaggtagatttgaggacagattggaattgaggtgaaagacagagaaagagaagtggccaggatga
ctccaagatttctgacctaaactactgggaaggacgcggttgtcatttctgaaatgcagaaggat
gccagaagagaaggtactttggggaggggcgggaatcaggagttagttttggacatgagataagc
ttggaatatttatttgctatctaagacagctccttaacatggtaagcccttatgcaagttgttgt
cagctgagatgggcgtggcactgagcatgggagcatggaggcgcctgagtggtctcatgctcagg
tggtttagcaaactcagtgtacatcctgccaattccagtcctgccatggccactgacaagctagg
agggcgctgaaaggagaaggaccccgatgtctcctccagcccatccatctcctctctcccattgg
ccaaacccaaccggaaactaaaggccaagggtacccggtgatgaagactgtggtatcagcctcct
gagcacagagagggcagaaaggggtggagacaaagaggggcgcagatagtgggcaaatggggaag
tggcacttcccctagctcgagggcagaggcttggtgtgatggaatggcactccttaaactgctac
atattttccctttaatttggccaagaacaagttgtcaagtttgtgtgagataaaggtgcacttgg
ttcgttcttgtctaatggcccccgcacccatgggtatttcttcagcttccacagtcatcccgaca
ctagctgggaagctccagcagccctggtcctggccccagctctgtgggcgctggccctcaacttt
gcctgcactgtgcttttgtgctattccccttggtcctgtttgggtgcaagtccccctcacgcatt
gagttcctgggccgctcaggctgctcctgtgtctccccagggaacatgaaggtcttgcaggagcc
cacctgcgtctccgactacatgagcatctctacttgcgagtggaagatgaatggtcccaccaatt
gcagcaccgagctccgcctgttgtaccagctggtttttctgctctccgagtaagcctgcgctgga
gctggaggtttggggaggttgtgcccaaagggtttgccccaagagtgagctgggtccaggtggtg
cgctggagtgcaggatgctgagtatggtttgctgctgtttatatggtgttagaggggaggtccca
tctccagggacatgttatgtaagatacagtggagcgcatggtgggagtgttggtccacgtggcac
atggatacggctggaatactggactagaccagcagttctcacactttttggtctcaggacccttt
ttcacacttaaaaatgagtgaggacccaaagggctttggtgtaggtaacacatcattctatgttt
acctaattagaacttgcaatgaagaaatggtgtaatttttaaaaaattaaaacaattaaaaattt
tttttcttactgaaatggaggtctcactgtgttgcccaggctgctctcaaactcctgggctccag
tgatcctcctgcctccgcctcccaaagtgctgggattacaagcgtgagccgctgtatccggccca
aaatggagaaattttaagtcccaacaacatgcaagcccgcattcaacaaatcttcagatcaatta
catgatcacaggtcatgtagcctctagaaaattccactgtacgccagtgagagagagtgaaaagg
caaataacgtccctgtattatgatgaaaagagttttacctggtgggcccagaccacactttgaga
accactggactagacccttgattgaggagtacggtgttgagagtggagtcctctgtgatggtgga
tggaccaggacacatggcataggagtcaggtggttccctgggctactccatggtgcacaggatgc
ttcgttacactggtgcccaggacataatcacgtacacaagacacacagttacggggcagactggg
gatatacggcacaccagcatgcagcgttcaccagtaaaggtggtattccatgattattctaaggt
agatgggctgtgctttgtttccattggcttagtccagggattggcaaactatggcccgtgagcca
aatccggcccactgcttgtttttgtaaataaagttttattggaacacactggctgctgtagttgt
aacagaaactgcatggccctcctttatgttttttgtttgtttgtttgtttgtttgttttctttga
gacagagtttcgctcttgttgcccaggctggagtgcagtggcacaatctcggctcactgcaacct
ctgcctcccgggttcaagcgattctcctgtctcagcctcccgagtagttgggattaatggtgcct
gccaccacacccggctaatttttcgtatttttagtagagaccggttttcatcatgttggccaagc
tggtctcgaactcctgaactcaggtgatccacccgcctcagcgtcccaaagtgctgggattacag
gcatgagccactgagcccggcctcctcctttatcttaattgaaataattcagaaatggaaagtca
aatactgcatgttctcacttataagtaagagttaaataatgtgtacacatgggcattattccatg
taccatggaataacagacattgaagacttgggagggtgggagaggggtgaaggaagagaagttac
ttaatgggcatagtgtacaccatttgggtgacggacccaccagaaccccagacttcaccactagg
cagcatatccagtgagaacagatctgaggcttgccatcaaaattgcacttgtaaggccgggcact
gtggtggctcgcggctgtaatcccagccctttgggaggccgaggtgggcagatcacttgaggtca
ggagttcgagaccggcctggccaacatggtgaagctccatctctactaaaaatacaacaattaac
tgggtgtagtggcgcacacctgtaatcccagctactagggaggctgaggcgggagaattgcttga
gcccaggaggtggaggttgcagtgagccgagatcacatcactgtactctagcctgggtgacagtg
agactttgtctcaggaaaaaaaaacaaaaacaaaaaacaaaaaactcgtaccccctaaatttata
caaataaccaaaaaaaaaaaaaaaaaaggaaattgtgtggcctttgaagtccaaaatattaacta
tctggcctgttacagaaaaagtttgcagacccctggcctagcccgtgagatgtgggttggctgtt
aaggtggaacattggaattatcttacgatggccaaactgtgcgatgcagagcttatgttgttcta
aattaattagtgccaccggttcttccctttcatgggctttcaggaacaagctaagtcccaggacc
agggccggcagctaggcaggtgtgaggagcatccttggtgcatgtggtaagaggctgtggccagc
aagagaggcaaccctagtcggctgccccagcacaccctggccgctcccaagcccccagatctgtc
ctcacatccgtgatcgggaagctggaagagtctgatgcggttcctggaggcatgtcccggacaca
gctgtggggcccagccagcctacaggtgaccagcctaacccagcccctgtgtctgcagagcccac
acgtgtatccctgagaacaacggaggcgcggggtgcgtgtgccacctgctcatggatgacgtggt
cagtgcggataactatacactggacctgtgggctgggcagcagctgctgtggaagggctccttca
agcccagcgagcatggtgagcagggcggagtgcggcaggggtggctgggtgtgttcccacagctg
cctgggctgaggggggggggcaggggaggaggtggggtcatagcaacagcaggaggaagccgcct
gtattttcccaaatctgatgggattcctgcccctgcctgggcctcagtcctcccacctttgaaac
ggagctggtcgcagtagaccaccaagcccccttcagcccagctgtttccacccctgaacttaagt
gcccaggaaggcgtattgagatgaggtgtgcttgctggaaggcatgcctgctgctgattgaaaac
cgaactgggaacattccttccattctgtgtccactggtcagctgctgcggctttggatggtcttg
accgtggaaggctgaccttcttctggtacccggagtccctgcaggaatcccccttgagcttgctg
ggctgtggtgacaggagtttaaaacatgcgttgtattccagtgatgcatgatatgacatgcatca
caggaataaaaacctgaggtctcatggatatgattgcttcaaaggagaccaagttttaaaacaga
tgaatcaaaataaagaaaaatactcagtaaatcatcataaagtacagagatgtggccaaaggtgt
gaaggatgcagctgtaaaagctgaagtttgaggccgggtgtggtggttcatgcctataatcccag
cactttgggaggccgagcccagcggatcaccggaggtcaggagttcgagaccagcctggacaaca
tggtaaaaccccgtctctactaaaaatacaaaaaattagtctggcatggtggcaggcgcctgtaa
tcccagctacttgggaggctgaggtaggagaatggcttgaacccaggagaaggaggttgcagtga
gcttagatcatgctactgccctccagcctgggcgacagagtgagattacgtctcaaaaaaaaaaa
ataaataaaaataaaaagattttttaaaaggctgaagtttgggttactttggctcatacactttg
ccttcactgtagaaaggtggttagtaaagaccaggcgcggtggctcatgcctggaatcccagcac
tttgggagcccagcgcaggcagatcacttgagccctgggctattgaggctgcagtgagctgggat
tgtgccactgcactccagcctgggcaacagagtgggaccctgtctcaaaaaagaagaaaaaaagg
gtaattaataaacactaaagttctatgtagaattttagcaacattattgttattataatcttctt
tgctatggctctgaatctgtgtggtgctccagaagtatgctatggaggttttgtcgaccaaaaat
ctgggtggtggctgtggtttgtaggccggggctgggctgggtgatgggggagtcactgcatagat
cctcacatagaggccgcttctcccgcagtgaaacccagggccccaggaaacctgacagttcacac
caatgtctccgacactctgctgctgacctggagcaacccgtatccccctgacaattacctgtata
atcatctcacctatgcagtcaacatttggagtgaaaacgacccggcagatgtgagtgggcatgct
ttgacgtttttctgtgacctctggggaacagggtgggtgaccagcagaggcccagtccctggagc
caggagcctgggaggcaagccctggggctggatagcaaatcccaggagctagagacctggcttct
cacctggctctgcactaggcaagtccctttgcttcctggccccccacccctcacatcagagaagg
ggagttatctctgcatgccgctcctcctctgtaaaggtagggctgtgggccacatctgtgtttcc
cagtttgggggacacaagtgatcgtaggtggcacattgacagctcacttgaataaccctattatt
gaagagaataatactgactcaagagacagtgacccgtgtcagttcccttttgaggccaacgggtt
aaggaggaagtccccatacagctgactcgtttactaattcctcttaatgaagagagcagaggcca
caccccaggcttagactttcccaagaaaacaagatcagtttgttggttgttccccatggaagctg
gtcctgacattcccttcacagtagtgttggtggagtttttgttgttgtttgttttgagacagagt
ctcactctgtcacccagggtggaacacagtggcgtgatcttggctcactgcaacctccgcctcct
gggttctagcgattctcctgcctcagcctcctgagcagccgggactacaggcacctgccaccgtg
cccagctaatttttgtatatttagtagagatggggtttcactgcgttggccaggctggtctcaaa
ctcctgacctcagatgatccactcgccttggcctcccaaagtgctgggattacaggtgtgagcca
ccgcacctggccagtggagttccttcttaagtacatgtattgacatctttaaaaagggcgagagg
atttacaggaaactatcaggtcagtaatggcaggggccgtccacagtgggtggctgagtccccct
atttttctgctggtgtgcagggaggtcatttcctgccacccatgtttccccaccctgaatccacc
ttcctcacattcccattggagggacaatctctggacatatgggacctggggtcccacagggctgc
aatccaatgcctgctgtgccactcgccagctgtgtgatgttgggcatatcccataacctctttgt
gcctcagtttcctcatctgtaacacaggagtgacaagagcacccgcccacagggctatgacagta
caaggtgtgtgatacagatgagctcccctgtttggcccacatgtgtcctaaaagccatgtgccct
ttctcttgagtgccccaggccacagagatccccatctgcccgctgtcccacacactggtctgtca
tttgttccttgaggtttgtgagggccggctctgtgcatcccaggggcccaggctgggcctggttg
gctctcagggagcaggcacccgccaccttaagctcccatgctggtgtctgtcactgcttcctctc
aatctggccaagccaggggtgtcgatttatatctctcaggtctggtttcccctttggcactgggc
caggtatggggaaagagcaggaatggggcagttggctcacacagcagaggctcagaaagcggggg
gcatggggggaaggagtgcacagatgctagagagtggggcaagttttgtttggtcaataaatctc
cttctcatgccccaggcctgtgcaagacctacagagagtcccaaggatgggctggggggaagaga
aaggtaccaccttcagagtccaaagatatgttatttaatattttcatatttctagatctgccttc
aggcatggctggatccagcttctaggaacctgtccagctctgcgccctgctttattctgtactgg
cttcgtttttaggcaggctcttccctcatgtagtggcagatatgcctactagttgctccaggcct
acatcccaaagccacagtgggaaaagggttttttttcttgacggttctaataagagtcctaaggc
tgctgctcagtggcctggcttcgatgctgtgccagcctctgaaccaatcactggctgtgggtgga
gagagggtgctggtggagggccctgcttgtccagggaggagtcacatacctgcctctagggctgc
aggtgggctcagctccatccaaaccagatgaactgaaaataaggcaggagtggcttccccagggg
aaactggggaagaggaagcaggactgtgctggctaaaatgccagccaggtttaagacgtggcacc
agatgccagtcatgggattggattggtcagcatgcctgggctatggcttaggggtatgttggtgc
tcagggatgccacaggcctccagataccaggtctgaggcagaagaatgaagtccagcttctcttg
tgggtggaacagtggcaactgagataccccatctctcccttcccaagaacagagctgaacataaa
gaatttagtgattggccagagcttggccacatgctcccctctgatgaatgataggccaggtgatg
ggattggcacaattggcttagactaatgagggttggccctggagttgcaggcagtggagttctgt
cctaagcagtgggcacctaaacccgatggcataaaagctgggcgggtgtccacctgcatctgcca
cagcactataggcaccaactgtggctcatactgagtgggataaattccagaaagaaacattagga
acttactatagaattttggggctagagctactcattcattcccctagataatttctaggcaaggt
tccatagtggagggggagttttggcttgggcattgaaggatgcataggagttttctagatgggga
aagaagggaacggtagaccaggcagagggaactgcatgataaaaggtttatgggtgtgaaaattc
atggaatgtttgaggattatggggttgggggatgtgggaatatgtgtagcgataaagcaccaaac
aaagccaaaagtttagttagagccctgaatgcctgcctcataatggtttccatattttatatgcc
tactatgtgccaggcacattgctcagggtcacacagctggaaatggcagggctgagtttttgttg
ttgttgttgttgttgagacagagtctcactctatcacccaggctggaatgcaggggcgtgatcat
ggctcactgcatccttgacttcctgggatcaggtgattctcccacctctgcctcccaggtagctg
ggactacaggcacaggccaccacgccaggctaattttttgtatttttagtagcgacagggtctcg
ccatgttgtccgggctggtctggatctcctggcttcaagtgatccccctggctcagcctcccaag
gtgctgggattacaggcttgagccaccgcatccagcccagatctgagatttgcacccagtatttg
aactcccaagcctgtgctctttttcctcccatggacatttctctcagagatggtctcccaaacac
ctgtccttcttgttaaaaaacagacaaaccgcaagtagttctttggaagctcagatttctctttt
gtttcttagtaaaacatttcccagttcccagctcccttccagggtgtaagatttcttcggtaact
tacatctagctgttgcttcttgtttgctcatgtttagaaagaaagacaaaagagagtgagaattt
tctctcccttccccagtctccccacaactcacaccccaccctcagctccctctgtaataggaaaa
tctctgaactctctgtagttgctccagcaatcttttggaactttgcttctttcttgtgaaaaaac
ctccccttggctcactttgcaccaggtttccccaaatgtgcttccaaccacaagcagaaatggag
ctgccagtaaccaggaagaaactgccgggggctgaggaagaggagagggaggtgcatagccctgg
atctcgcagggagaggggtgacaggatgagaactcaggttgctcacttgccatcagggtcagtca
tgaatatagcgttcatgtatcactttttaaagcttttttggagggtaaaagtaatagttacacaa
aataaaaatacaaatggtacaaaaggacttagaatggaaacatgtttctctcccgactccagcct
cctgtttttcttcccagagactgaccactgctgtctgtctcttgccagaagggaaagggaggcaa
ggttagggcaggcagagggcatgtgcatcctttagagagagcttatgtctatacaagcaaatgtg
tgtgttcagtcatcgctgtcttagttttctattgctgcataataatggtactaccagcttcacag
ctttaaacaacacccatttattatctcatagtttctgtggttgggagtctggacatagcttagcc
aggttctctgctttagagtctcgtgaggctataatcaaggtgtgggatggggctgcagtttcatc
tgaggctcaattggggaagggtcacttctaagctcatacaatattggtgacattcagtccctggc
aggctgttgaactgagagcctcagtttcgtgctggctgttggttgtagttaaccctgaattcctt
cccatgtgccctttgcaaagccatcaaggcagagagacttgcctagcaagtaggatattacagtc
ttctgtaatataatcacatccatgaaatcctctatatatcccatcacctttaccatattctgtgg
gttagaaacaagtagcaggtcctgcccacactcgagaagaccagatgacacaaagatgtgattca
aagtggggatcatcggggccatcttaggtttgtctgcagtgatcactgtgccatctctctctctc
tctttttttttttttttttttccgagacgaagtcgtcactctgtcacccaggctggagtgcagtg
gcatgatctcagcttaccacaatctctgcctcccaggttcaaatgattcttctgcctcagcctcc
tgagtagctgggattacaggtgcccgccaccacacccagctaatttttgtatttttagtagagac
agagtttcaccatgttggccaggctggtcttgaactcctcacctcaagtgatccacccacttcgg
cctcccaaagtgctgggattacaggcatgagccaccatgcccagccccatctctctttaaaaaac
aaacaaacaaacaaaaaacataaaaagaagcagagaacacatacacatctgcatcttcccttgtt
tacttaacaatagatcttggaagtcacttctcagtagaggctaggttgggcagagcattggattc
taggccagtgagtttggacttgaccatggagacactaggaagcccatgaaggacagagagagatg
cctcgaccctgccagtcctttagaaagatcacccagtgctttttgtataccaaaccctatttgaa
atacttacgtatattaacccatttccttatcaccacaaccctgcgggaagggagataggcacttt
tattatcttcattttgcagatgaggacattgaggtccagagaggttatgtcacttacttaaggtc
acacagccaggaagtggtagtagggactcttacccttgttttacagatgagattgaattatctca
cgaaaactcagaaaggttaaacaacttgcctaagtaacatacagctaattagtcgaggagcctga
cgcatgttgctgtagcctggtcacagttacagaggtggcaagcaatggcctgaacaggacgaaca
accaaatacccaggctggtggctcttaaacatggtggggtcagctaacgacagcaaccagggggg
cactggtgcccctcgcccccggctggtgccctaacatctcccttttctctaccagttcagaatct
ataacgtgacctacctagaaccctccctccgcatcgcagccagcaccctgaagtctgggatttcc
tacagggcacgggtgagggcctgggctcagtgctataacaccacctggagtgagtggagccccag
caccaagtggcacaactgtgagtatcaagaggcctaagcaatggtaatctccactctccattctt
cccctgtggccagacacttcccctggctgagtctctgggcttttatatcataggatgcctctaat
ggcaatcctgccattagatacacctgccgtggtgtatctgccaggtaggcaggctaggctgcagt
aacaaacaagcccacaatttccatggcttaacactatgggaatatatttcttgctcacgtaacaa
gctaacgtgaatgttgctggtttgtaggtggtttccctccctgtagaaatctggggagtgaggtt
ctttccatcttgtggtgccgtcattctccaggacaaagattcttacctacttttgtgtcctggtt
tcctttggcagcctggtgaagcctatggacctcatttcagaatatttttaaatacataaaatccc
agcctgggcaatatagtgaaacccccatctgtacaaaaattagccaggcatggtggcatgcacct
gtagtcccaggtactgggaaggctgaggtgggaggatcacttgagcccaggagtttgaggctgca
gtgagccgtgatcgtaccactttactcccacctgggtgacagagcaagagcccatctctaaaaat
aaataaatacaatgaaataaaataaaataaatagaactacagaggaaactaattgtattgaaatg
cagttataaaacatttaaacacatttttaatctagagatatatgtgcttctttattaagatctat
aaataataagttctaggggtagctcgcataaatactgtaatttcaaagtagataagcataaataa
tactttatgatactgaaattgtgatgtgatatgagaatagctgtgagttttgttttgctggggac
aggatcagtgatgctgtcattactggggtctcttccctccattctttttttaaaattgtatttta
ttttatttttaaaattttaaaataaatagagacagggtatcactatgttgcccaggctgcttttg
acctcctgggctccagtgatcttcccatcttggcttcccaaagtgctgggattacaagtgggagc
cagtgttcctggccccttcctccattcttaatggaaggagatgctaggtgtgagaggttagggaa
agtaaagatgtaatttctttcccatccaagttctcagacccctgaattctacctgcagccatgtt
ggtccatcaaccccaagtgaagaatccctgctctagggccccaccattgtctgtatccagccagc
agaagaggcgtgattatggagatcacatctgcttcttgaaagcagacagcccggaagtgggccgc
atcacttcctctcaaattctattggtgaaaatggtcacatgactacacatagccacaaaggaggc
tgggaactttctcacttggaacctacatcccagaaacaactcttttcagtgaggtatcccacagg
tctttcgcagtagaaatattgattatctcacataaaatgaagtcttacaaatggacctactgggt
tttgtacagcagccaagtgatatctcttcccttctgctgtcttcccttctgccatccttcacatg
gtggcattgtatccttagacttgccacccatgccctcaggttggccgttgcacactgtcttacat
aaagcaggaaggaaaggaaaggctgctacgagagagtgtaccttgtgcatctcttttttaatcag
gaagcaaacatctttctagaagcttccctagcaaaattccccttacatctcattggccaagactg
ttacatgttacatggttactgttattacttgctcattgcaaggaagactgggaactcaaatgcct
ggaaaaaggaacaggataatcgtgattggctcaagccttaggggggcatggctccctgacaaggg
agagaggaaaaagctgttgagtgaagaagactgcttcagtttccccatctgtataatgggaggag
taagggctgtcgtaaaaactcaatgaaagaagattcttcaacgtggtaggtgcagtggcagctgg
cagtaccctgaccctgccaccgcacagccctctcagcattgctcatcctgcactgtggatatcag
ttgagccacgtgtctcctgccctgggctgtgagctccataggcagggtctccatggctgtatctc
cagaacccagcacagaaccaggtgcttgggaaagttttgaattgattctcatctgccattggcat
ggggaagggaactagcttgtatgaaacagataacaatgtatgggaccctcattcattatttcagc
aaatatttgctgagttcctcctacatggctagccctgtgctagacactggggaatcggcgatgaa
caaagcagatagaaatccccactcttgtggagctgacattctggagggagagacaaaaagcaaac
atataaagaaagaaagaaatcacatggatctggatgacagtgagtgctgggaagaaaataaaagc
agaggaaggggatggagcgatgggcagggggcaacggtagggagggtgtcggggaaaactttttg
gagaatgtgacgatgaaagtgaacaaggagaagtcaaccgtgttgagatgatggcagctaatgat
gtggacaggccactctgttctgagtgcattatctattgattcatcatgtcatcctcgcaacagcc
ctgcacgatcaattctgtcattaaccccatagtacagatgaggatgcggaggcacagagaagata
agggacttgtcctgtgtcacacagcaaggagccatccggctcctaagttggtgcatttgacttct
gtgcttccggaaagaaagagcagcaagtttaagatctggaggtggcactgagctttggaggagca
gggggcaatgaggtggccggtgtgacgaggactcaatgtgcaagagggagagtggtggggagatg
aggtggaggggtggtcggcggtcagatcgtggagggtctcggacgagggtcctgaccctgggtct
ccagtcctgggaagtggagcccaggctgtaccatggctgacctcagctcatggcttcccctccca
cttccagcctacagggagcccttcgagcagcacctcctgctgggcgtcagcgtttcctgcattgt
catcctggccgtctgcctgttgtgctatgtcagcatcaccaagtgagtcctgggcccagtgctgc
cgagcagtccctctggagtgcagggtggcagggacttgcccctctagtctgcccctttgcagtcc
tctcagtcaataatacgcatttactgagcagctactacacaccttgagagtagagctgagaacat
atcgacaaggaccccacttttttctttttttttttttttttttttttttgagacggagtctcact
ctgtcacccaggctggagtatagtggcacaatcttgcctaacagtaacctccgcctcccgggttc
aagcaattcttctgcctcagcctccagagtagctgggattacaggcgcatgccactatgcccggc
taattttttgtatttttggtagagatggggtttcaccatgttggtcaggctggtctcgaactcct
gacctcatgatctgcctgcctcagcctcccaaagtgctgggattacaggtgtgagccactgcacc
caaccaggactccacatttctaaaaccggcatcctactggggagactgaaaatacatatcaatca
caaacaggtggttttccatagtgacccactctctgaatgcactagaccagggtgcaggccagaga
tcttctggggtgctttttgcaagggggaccaggataaggctctccaaggagggaaaatttgaggg
gggccctgactggggagaatgagctggccagggataagcaagatggagtcatcccacatcccctt
acaacgctgggtgcctgggcaactgggggcatctgggggcatgtggtaggagccagaggaatttg
cgacgattgccctgatggagtcaggagacctgggtttgaatcctggccttggagcttggtagctg
gcggccgacaagttgctgaaacccctgagcctggggttcctgctttgcagagtgacagtgatggt
gagaacatatttcatcagccagaagaggccaaatcacagtaaaggctgagggaggagatgagtgg
cgagtggctgggaggtggtggaaggagcctcgtttccagagagctcttgccagcccttggaatca
tggtgtctcagagcctcagtcctcccatctctgaaatgggactagcaagctcaacctcactaagt
caggattagaggtggctaaggattattaacatgattgatgaaagtgcccactcttggcccagcac
acactaggtaggcagggaatgcaaattcccctccatatcttgtcactgatgcctccgagcaacct
tggactgatcgccttgctctgagcctcagtttccccatcacctgtacctcttcccactccccatc
actatatcccagcatgccagcctctttgctgttctttgtctttggtttcttgttttgttctgttt
tttagacagggtctcactctgttagccaggctgaagtgcagtggcgcggttacggctcactgcag
cctccaattcctgggctaaagagatcctcccatttcaacttccagagcagctgggacaacaggcg
cttgccaccacacctggctaattttcttattttaatttaatttaattttattttttgggacagag
tggagtctcaaaaaccaagctggagtgcagtggtgcgatctcgactcactgcaatctctgcctcc
cgggttcaagcgattctcctgccttagcctcccgactagctgggattacaggcgtgtgccacgac
acccagctaatttttgtatttttagtagagatggggtttcaccatgttggccaggatggtcttga
actcctgacctcaagtgatccacccacctcgttctcccaaggtgctgggtacaggcatgagccac
tgtgcctggccaattttcttacattttgtagagactggctgtcacttatgtagcccaggctgatc
ttgaacttctacccctttatctttattcatggcacttattaccatgaatgaatgacctcatataa
gcatttctttcgtttttttttttttttctttgagatggagtctcatgttgtcccccaggctggag
tgcagtggcgcgatctcagctcactgcaacctccgccttccgggttcaagcgattctcctgcctc
agcctcctgagtagctgggattgcaggcgcctgccaccatgcctggctaagttttgcatttttag
tagagacggtgtttcaccatattggccaggctggtctcgaacttctgacctcaggtgatacacct
gccttggcctcccaaagtgctgggattacaggcgtgagccaccatgcctggcctcatataagcat
ttctgtctccatttatcatccatctttccctcttgaaggtcagtttcaccaaggcaggcatcttt
gtctcgttcactgttgtggcctcagggccaggcacagtgagtcaaacatagaaggtgctcaataa
atatgtgtttatttattgaaaccatgggcagaggctaattcagaagcggtctgaggaccttacct
cccagtgatgatgcaccatggccccaggcaggccaggaagagagaagggttgtgtttctccgtag
gtcccccagcttcccaggccatcccaggccattccctggtcatttgccctcagctgctctgaaaa
agggattgttgaggggaacctagaatcctctctctgcagtttgagtctttcctaatcccctgggg
tctcattcccactgaggacataggtggcctcctcaggaactctgtgctgggtaacagaatgcggg
agtgtgaacctggctctgccacctaccagctgtcactccacctccttgggcctcactctcctcat
ctgtagaatagggttagcaatagaatccatgtcaccaggttagaatgatgagtcagtggtttgac
ctccagaaactaatcagcctgatctctgatgccaaataagtattggtgataacgaccacttttat
gggaggagcgttcacctgtcaataattcagagatcaacaccttttccttttgtttttcaggatta
agaaagaatggtgggatcagattcccaacccagcccgcagccgcctcgtggctataataatccag
gatgctcaggtaggagtaggcgtggatgaggacatgtgggactgtgtacatgaagaagtgtggtt
cagaacacctgggctgttaaggaccttcactggcttctggaatggcaaatagacagtcaggaggg
ttgcaggggagacagagacagaagccgaatgaggtcattagcagaccagaggctttcccgccctt
ccccttggcaatcccagcctgggggggcttctctggggttggtttcctgtttttttccctcccct
tgggagaatgacccttgggtcatcatcactgtgtcattccctggggaggtgccagtaccagggct
agaggccagaaggagtggaggaaggagagggtgacaggctttctgtgtcttcttcttaagcatag
gaaactgcccccgaagcactagcaaatcccttccgggttctcattggcctgaaatgtatcccacc
cctaagccaggggggagtcagcttccccaaggcgatggtcctgtgggtgagtgggtggggtttgc
ctgagcaagatgagagttctctaggtaggagaaagggggattataggtcctgtctagaagagaag
gtctgagggtccttgcttttccagggactctggaatctagtggtgttggctttgaatcctgactc
tgccactcactggcagtgtggacttgagcaagttgcttaattctctgagcctcagtttcctcttg
tgggttataacagtgtttacctggtaggacagatattggaatttattgagacaatacatataaag
tgcatattccagcctcttgcaaataccaagtgccatttatgtatcagttagtgtttgctgtgtaa
caaacgaccccgaaatgtagagggttacaacaactttatttagcttatgcttctgcaggctggca
tttggggctgggctcagcagtgagggtggcgggggaggctgggctgggctgggctgggcagatct
gaattgagctgacccgtccccgtagcctccctccgtgtctgacagttggcttttttttttttttt
ctttttctgagacggagttttgctcttattgcccaggagtgcaatggcgtgatcttggctcactg
caacctctgcttcctgggttcaagcaattttcttgcctcagcctcccaagtagctgggattacag
gcatgtgccaccacgccaggctaattttgtatttttaatagagatggggtttcttcatgtcggtc
aggctggtctggaactcctaatatcaggtgatccacccacttcagcctcccaaagtgctgggatt
acaggcgtgagccactgcacccagcctagttggctgacttttacctgggacagtgcaggtgcctg
agccatgtgcctctcactctccagcaggccggcccaggcttgtttacagagtggctcagttttca
aggggggaagtcccaaggcttcttgaggcctaggcgcagcactggcatgatatcacttccatcac
attctatgggcccaagcaagtcccagggccagtgtagattcaagggatgggaggagattcagagc
actcctctgtggccacttttgccatcgaccacagtccctgtaaatattaggacaatgtaattaat
tcccaggaatctgaagctcagaaagcgtaagtgacctgttggacttctgatctgtgtgatgtcga
ggcttgtaccccttcctgagcattgccgtactccaggccgggctgcaaggccactctgctctttc
attggctgtctctgtattttaggggtcacagtgggagaagcggtcccgaggccaggaaccagcca
agtgcccgtatgtatctgaacttaggtcacagcctgcatgcattgggaaggtgatagaattggag
aggcaagcccctagctccatgtctgccttctcttccctgcattcggtaattgccctgtgacatta
gccttcaagggacggcaggaggaggggtgttctggaaacgtggactgctggccaagccccctgag
tttcactggtgtgtcaggtacatggtgataccccttgggagtgctgttatagttaacaaccagag
cagccgtgcctgttgttaaaatcttgacctaattgtatacttgtcggcaaatagccactatcctg
aacactcccctccttttttttaatatacaggatctcactctgtggcccaggctggtgtgcagtgg
tgcgatcatagctcactgcaccttcaaactcctgagctcaagtgatcctcccatcttagcctccc
gagtagctgatactacagatgtgcattaccacgcctggctattttaaaaggtttttgcctgtaat
tccagctactcaggaggctgaggcatgagaatcacttgaacccgggaggcagaggttgcagtgag
cgcagattgtgccactgcactccagcctgggcgacagagtgagactcttgtctcaaaaaaaataa
taccaaaaaaagtttttgtaaagacaagctctcgctgtgttgccccgccactgtggcctccttag
cttcttccctggggcctgctggacctttccatactccagaaactaaagggggtccaggaccctgc
ttcaaccctaggatcccgcatcttttttttttttttttttttttggacgcagggtcttgctgtgt
ccctcaggctggagtgcagtgattcactgcagcctcaaactcgtgggctcaagtgattctctagc
ctcagccttctaagtagctgggactacagtcatacaccaacatgcccagctaattttcctttttt
ttaattcttgtagagatgtttgagacggcttgggctctgttgcccaggctgttctcaaactcctg
agctcaagcgatcctccctcctcagcctcctaaagtgctgggattacaggcgtgagccaccgcac
ccggcttccatatcctttctaattggtcatggcttgggataatggtgttgcttttaattatcatc
atccataaagactttttcttactcaacagatctgagcttgtatttggtgcccaggacatgtgctg
ggttcccgaaatcccaaagacacagaccctaccctcagggatttctcattctagcaacatagact
gatcaattactgattataacgttagaaggcatgtctgaagtagacagccatcaggacatggtgat
ttcaggctgggctttgaagaatgaataggagtttttcaagtgtcgaaactgaaccctgaccaacc
tttgcttttgcagacactggaagaattgtcttaccaagctcttgccctgttttctggagcacaac
atgaaaagggatgaagatcctcacaaggctgccaaagagatgcctttccagggctctggaaaatc
agcatggtgcccagtggagatcagcaagacagtcctctggccagagagcatcagcgtggtgcgat
gtgtggagttgtttgaggccccggtggagtgtgaggaggaggaggaggtagaggaagaaaaaggg
agcttctgtgcatcgcctgagagcagcagggatgacttccaggagggaagggagggcattgtggc
ccggctaacagagagcctgttcctggacctgctcggagaggagaatgggggcttttgccagcagg
acatgggggagtcatgccttcttccaccttcgggaagtacgagtgctcacatgccctgggatgag
ttcccaagtgcagggcccaaggaggcacctccctggggcaaggagcagcctctccacctggagcc
aagtcctcctgccagcccgacccagagtccagacaacctgacttgcacagagacgcccctcgtca
tcgcaggcaaccctgcttaccgcagcttcagcaactccctgagccagtcaccgtgtcccagagag
ctgggtccagacccactgctggccagacacctggaggaagtagaacccgagatgccctgtgtccc
ccagctctctgagccaaccactgtgccccaacctgagccagaaacctgggagcagatcctccgcc
gaaatgtcctccagcatggggcagctgcagcccccgtctcggcccccaccagtggctatcaggag
tttgtacatgcggtggagcagggtggcacccaggccagtgcggtggtgggcttgggtcccccagg
agaggctggttacaaggccttctcaagcctgcttgccagcagtgctgtgtccccagagaaatgtg
ggtttggggctagcagtggggaagaggggtataagcctttccaagacctcattcctggctgccct
ggggaccctgccccagtccctgtccccttgttcacctttggactggacagggagccacctcgcag
tccgcagagctcacatctcccaagcagctccccagagcacctgggtctggagccgggggaaaagg
tagaggacatgccaaagcccccacttccccaggagcaggccacagacccccttgtggacagcctg
ggcagtggcattgtctactcagcccttacctgccacctgtgcggccacctgaaacagtgtcatgg
ccaggaggatggtggccagacccctgtcatggccagtccttgctgtggctgctgctgtggagaca
ggtcctcgccccctacaacccccctgagggccccagacccctctccaggtggggttccactggag
gccagtctgtgtccggcctccctggcaccctcgggcatctcagagaagagtaaatcctcatcatc
cttccatcctgcccctggcaatgctcagagctcaagccagacccccaaaatcgtgaactttgtct
ccgtgggacccacatacatgagggtctcttaggtgcatgtcctcttgttgctgagtctgcagatg
aggactagggcttatccatgcctgggaaatgccacctcctggaaggcagccaggctggcagattt
ccaaaagacttgaagaaccatggtatgaaggtgattggccccactgacgttggcctaacactggg
ctgcagagactggaccccgcccagcattgggctgggctcgccacatcccatgagagtagagggca
ctgggtcgccgtgccccacggcaggcccctgcaggaaaactgaggcccttgggcacctcgacttg
tgaacgagttgttggctgctccctccacagcttctgcagcagactgtccctgttgtaactgccca
aggcatgttttgcccaccagatcatggcccacgtggaggcccacctgcctctgtctcactgaact
agaagccgagcctagaaactaacacagccatcaagggaatgacttgggcggccttgggaaatcga
tgagaaattgaacttcagggagggtggtcattgcctagaggtgctcattcatttaacagagcttc
cttaggttgatgctggaggcagaatcccggctgtcaaggggtgttcagttaaggggagcaacaga
ggacatgaaaaattgctatgactaaagcagggacaatttgctgccaaacacccatgcccagctgt
atggctgggggctcctcgtatgcatggaacccccagaataaatatgctcagccaccctgtgggcc
gggcaatccagacagcaggcataaggcaccagttaccctgcatgttggcccagacctcaggtgct
agggaaggcgggaaccttgggttgagtaatgctcgtctgtgtgttttagtttcatcacctgttat
ctgtgtttgctgaggagagtggaacagaaggggtggagttttgtataaataaagtttctttgtct
cttta

The DNA sequences in this specification are provided in the 5′ to 3′ direction (i.e. “sense strand”, or “coding strand”). Since the antisense strand of DNA is used as a template for transcription, the mRNA sequence would have the same sequence as the coding strand, but with thymine (T) replaced with uracil (U).

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A splice-switching oligonucleotide (SSO) that binds to a IL-4Rα pre-mRNA, wherein the SSO comprises a sequence selected from the group consisting of SEQ ID NOs 1 to 8, and wherein binding of the SSO induces the exclusion of an exon during splicing of the IL-4Rα pre-mRNA to a IL-4Rα mature mRNA.

2. The SSO according to claim 1, wherein at least one of the nucleotides of the SSO is chemically modified and wherein the chemical modification is 2′-O-methyl RNA modification, 2′-O-methoxyethyl RNA modification or phosphorothioate linkage.

3. The SSO according to claim 1 or 2, wherein each nucleotide of the SSO comprises either a 2′-O-methyl RNA modification or a 2′-O-methoxyethyl RNA modification.

4. The SSO according to any one of the preceding claims, comprising phosphorothioate linkages between all nucleotides of the SSO.

5. An SSO according to any one of the preceding claims for use as a medicament or in therapy.

6. The SSO according to claim 5 for use in the treatment of a Th2-mediated inflammatory disease.

7. The SSO according to claim 6, wherein the Th2-mediated inflammatory disease is selected from the group consisting of atopic dermatitis, asthma, allergic rhinitis, allergic conjunctivitis and ulcerative colitis.

8. Use of an SSO according to any one of claims 1 to 4 in the manufacture of a medicament for treating a Th2-mediated inflammatory disease.

9. The use according to claim 8, wherein the Th2-mediated inflammatory disease is selected from the group consisting of atopic dermatitis, asthma, allergic rhinitis, allergic conjunctivitis and ulcerative colitis.

10. A method of treating a Th2-mediated inflammatory disease comprising administering to a subject a composition comprising an SSO according to any one of claims 1 to 4.

11. The method according to claim 10, wherein the Th2-mediated inflammatory disease is selected from the group consisting of atopic dermatitis, asthma, allergic rhinitis, allergic conjunctivitis and ulcerative colitis.

12. A pharmaceutical composition comprising (a) a therapeutically effective amount of an SSO according to any one of claims 1 to 4 and (b) one or more pharmaceutically acceptable carriers and/or diluents.

13. A method of exon-skipping comprising providing an SSO having a sequence selected from the group consisting of SEQ ID NOs 1 to 8, wherein the binding of the SSO to a IL-4Rα pre-mRNA induces the exclusion of an exon during splicing of the IL-4Rα pre-mRNA to a IL-4Rα mature mRNA.

14. The method according to claim 13, wherein the exclusion of the exon causes a reduction in the levels of the IL-4Rα mature mRNA or functional protein.

15. The method according to claim 13 or 14, comprising providing an SSO with at least one of the nucleotides of the SSO being chemically modified and wherein the chemical modification is 2′-O-methyl RNA modification, 2′-O-methoxyethyl RNA modification or phosphorothioate linkage.

16. The method according to any one of claims 13 to 15, comprising providing an SSO in which each nucleotide of the SSO comprises either a 2′-O-methyl RNA modification or a 2′-O-methoxyethyl RNA modification.

17. The method according to any one of claims 13 to 16, comprising providing an SSO comprising phosphorothioate linkages between all nucleotides of the SSO.


SEQUENCE LISTING (38 sequences)

SEQ ID NO: 1 - RNA, 22 nt, synthetic construct
  source 1..22: mol_type = other RNA
gggctcctgcaagaccttcatg

SEQ ID NO: 2 - RNA, 24 nt, synthetic construct
  source 1..24: mol_type = other RNA
ttgttctcagggatacacgtgtgg

SEQ ID NO: 3 - RNA, 22 nt, synthetic construct
  source 1..22: mol_type = other RNA
ggctcctgcaagaccttcatgt

SEQ ID NO: 4 - RNA, 24 nt, synthetic construct
  source 1..24: mol_type = other RNA
gtaattgtcagggggatacgggtt

SEQ ID NO: 5 - RNA, 30 nt, synthetic construct
  source 1..30: mol_type = other RNA
gttctaggtaggtcacgttatagattctga

SEQ ID NO: 6 - RNA, 18 nt, synthetic construct
  source 1..18: mol_type = other RNA
gctgcgatgcggagggag

SEQ ID NO: 7 - RNA, 21 nt, synthetic construct
  source 1..21: mol_type = other RNA
cgaagggctccctgtaggctg

SEQ ID NO: 8 - RNA, 23 nt, synthetic construct
  source 1..23: mol_type = other RNA
ccaggactcacttggtgatgctg

SEQ ID NO: 9 - RNA, 23 nt, synthetic construct
  source 1..23: mol_type = other RNA
attgggagatgccaaggcacctg

SEQ ID NO: 10 - RNA, 19 nt, synthetic construct
  source 1..19: mol_type = other RNA
cagggtgctggctgcgatg

SEQ ID NO: 11 - RNA, 32 nt, synthetic construct
  source 1..32: mol_type = other RNA
tatgatttcttccagctgtgtgtaaatcttta

SEQ ID NO: 12 - RNA, 27 nt, synthetic construct
  source 1..27: mol_type = other RNA
aaagtgctgggattataggcatgagcc

SEQ ID NO: 13 - DNA, 20 bp, synthetic construct
  source 1..20: mol_type = other DNA
ggcgcgcagataattaaaga

SEQ ID NO: 14 - DNA, 20 bp, synthetic construct
  source 1..20: mol_type = other DNA
agcccacaggtccagtgtat

SEQ ID NO: 15 - DNA, 18 bp, synthetic construct
  source 1..18: mol_type = other DNA
atggggtggctttgctct

SEQ ID NO: 16 - DNA, 20 bp, synthetic construct
  source 1..20: mol_type = other DNA
cgcgggccagggaagggcca

SEQ ID NO: 17 - DNA, 20 bp, synthetic construct
  source 1..20: mol_type = other DNA
ctccactcactccaggtggt

SEQ ID NO: 18 - DNA, 20 bp, synthetic construct
  source 1..20: mol_type = other DNA
ctcatggatgacgtggtcag

SEQ ID NO: 19 - DNA, 20 bp, synthetic construct
  source 1..20: mol_type = other DNA
gacactctgctgctgacctg

SEQ ID NO: 20 - DNA, 20 bp, synthetic construct
  source 1..20: mol_type = other DNA
ttcttccagtgtgggcactt

SEQ ID NO: 21 - DNA, 20 bp, synthetic construct
  source 1..20: mol_type = other DNA
accacctggagtgagtggag

SEQ ID NO: 22 - DNA, 20 bp, synthetic construct
  source 1..20: mol_type = other DNA
ctggaaaggcatctctttgg

SEQ ID NO: 23 - DNA, 19 bp, synthetic construct
  source 1..19: mol_type = other DNA
ttcctgcattgtcatcctg

SEQ ID NO: 24 - DNA, 19 bp, synthetic construct
  source 1..19: mol_type = other DNA
ctgggttgggaatctgatc

SEQ ID NO: 25 - DNA, 20 bp, synthetic construct
  source 1..20: mol_type = other DNA
caagctcttgccctgttttc

SEQ ID NO: 26 - DNA, 20 bp, synthetic construct
  source 1..20: mol_type = other DNA
tgcacagaagctcccttttt

SEQ ID NO: 27 - DNA, 20 bp, synthetic construct
  source 1..20: mol_type = other DNA
tgctgaggatttggaaaggg

SEQ ID NO: 28 - DNA, 21 bp, synthetic construct
  source 1..21: mol_type = other DNA
acagagggctacaatgtgatg

SEQ ID NO: 29 - DNA, 19 bp, synthetic construct
  source 1..19: mol_type = other DNA
gcaaattccatggcaccgt

SEQ ID NO: 30 - DNA, 20 bp, synthetic construct
  source 1..20: mol_type = other DNA
gccccacttgattttggagg

SEQ ID NO: 31 - DNA, 233 bp, Homo sapiens
  source 1..233: mol_type = genomic DNA
gggaattggctcttttcttcattttcttaaattaaactttttattttcagataattaaagatttacacacagctggaagaaatcatagagaagccgggcgtggtggctcatgcctataatcccagcacttttggaggctgaggcgggcagatcacttgagatcaggagttcgagaccagcctggtcagtatgatgaaaccccatctctactaaaaatacaaaaattagttggg

SEQ ID NO: 32 - DNA, 188 bp, Homo sapiens
  source 1..188: mol_type = genomic DNA
ctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgcc

SEQ ID NO: 33 - DNA, 239 bp, Homo sapiens
  source 1..239: mol_type = genomic DNA
ctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtt

SEQ ID NO: 34 - DNA, 252 bp, Homo sapiens
  source 1..252: mol_type = genomic DNA
gcccagccagcctacaggtgaccagcctaacccagcccctgtgtctgcagagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctg

SEQ ID NO: 35 - DNA, 252 bp, Homo sapiens
  source 1..252: mol_type = genomic DNA
atgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtg

SEQ ID NO: 36 - DNA, 257 bp, Homo sapiens
  source 1..257: mol_type = genomic DNA
gtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcc

SEQ ID NO: 37 - DNA, 200 bp, Homo sapiens
  source 1..200: mol_type = genomic DNA
aggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtg

SEQ ID NO: 38 - DNA, 51111 bp, Homo sapiens
  source 1..51111: mol_type = genomic DNA
gcagagttctccgctgggcgtgacctcgggctacggcgtgggaggaagcgcgcggcaagacacccagcgaggtgctggggtcgcccccaggagaggacggcggctcggactgtccggcggcggcggcggggacagcgacaggggcgcgaggtggccgggacccgggccgggcgcgccgggcggggcggcgcatgcaaatctgccgggcgccggggcggggagcaggaagccggggcgggctgggtctccgcgcccaggaaagccccgcgcggcgcgggccagggaagggccacccaggggtcccccacttcccgcttgggcgcccggacggcgaatggagcaggggcgcgcaggtaggatccggggcccgcgcgcggatcgggttgcgaaggtatcgcccgggcacgccggctgagggcgttcgggaagggctcggccgccggcggggaccacggggaccaccccgactccgagcggggcccgagcccgcgactctcggtgcgcgcggagcagcgcccggttccgtccttgccgccgaacggcagcggaggcgcgaggcccggggtacgtggacacccagcgctccccaaagccggtgctggcagtgagacctccgccgggacggcctgcgggggtggggggcttggggttaggctgtcggaggccacgcagcccctcttctccgggcagtggcgcccagccctgcgctcaggaagtcagtgaggacttcggagagagaaagggtggggaaagttctgagaactgtaaatttgagtagtaggtcagtgaatcggggcggtctccgcctccgaatatcagatggacccaataatcgcgatcattgactgggacttgttttactgaaaggatgccaagtgaaacctcccactaacttctctgtggccggtgctgtgcgtcatggacattgtacagatgaggtcaccaagaccccagccgctttagtaacttgccctaaggtgccgcccacttgggagtccgtggggcagcaggactagagcccaggcaatcccacgccagagccagctgctctccatctctcaatcacgtttgaggagcccccacaataaccagaatctccgggagatttgcttaaaatgcagattcccaggtccttgccctgagattcagattcagtaaacccaggaatctgatctttattttatttttaaaaatttattttataaagatggtgtctcactatgttgtccaggctggcctcaagtgatcctcccacctcggcctcccaaaatgctgggatgacaggtgtgagccactgagccctgattttttttttaaacaaaatctggatttcttagacttaagcaagataggggaccactagtttgtctgcttctggggttcaaggaatgcccccaggccagcgctaaccctgctccagttctagtctctccccttccccagccctctgggaggcatgacccactgcacttccctgcccccagtagcctatgaggagcccaaggcatttgctgggacctcagtgcctttatcaaaatcccaaagtccaatggggaaacaggctcaggtgtaggtttggccagggtgtcctgcacattagggcattccagggtcctgcaggggtttcttagaactcggctagcccagcccatgaatgggaggtgacctgctaagccactcattcactcactagcactcattcagctcctgcgaggccctgggtggtgttctgggcactggtgcttgcctcaaggagcaccccctctgcaggaagacagacgtgagggctgggagggtgcggatgcaccagcggctgcaggaccacaagccagaggggatcagctcttcaaaggtcactgggccaggcttcccagcacccagggaccctgagctagccttgtgagtagagcaggggagtctggcaggtagaggatgggaggtaaagggaggcacagagtgggcggaggctgggaagtgtgaaagggcactgtgtacagaacctagggagtaagcagaagccagagatggatggaaagttgggctgggctgcattgtggaaagccttgaatgccatgccaaggaacttgggccagtgtattcaccttggcttgagttgtaaaagcaacaatgcagattcctgggccccaccctgggcctctggatctgaatctggagggagacgctggggaggcagcagggaggaaaacctgcagtcactcaccccatttacaacacatgcagttaccggcaggtccgagataacctaagggtttccaaatgctaggaagtgactcaatgactgatatgtttaatacaatacaatcccggttgcaaaatgcccagggcaagggtgggggtgttcagttttctaatgctcccagttcaaaaaggagctgaaatgaattgtcaaagaagtgacaagctgggcgcagtgactcacacctgtaatcccagcactttgggaggctgagagaggatcgcttgaacccaggagtttgagaccagcctgggcaacatagtgagatcctgttcctacaaaaaaaatttaaaaacttagccaggcgtggtagcaggtgcctctagccccagctatttgggaggctgaggtgggaggatggcttgagcccaggtggtcaaggctgcagtgaactatgattgtgccactgcactgtagcctgggtgacagagcgagactctgtctcaaaataaacaaatacataaatacatgagaagagagaaccttttctacctcccccatctccctgccctcttccacccaccccatctgggacgatggaggagagcgtttgggattagatgccccacctctcgactgccctcactggtacctgggagtgaggccaggacagaagccgcctaagtactgttcttcctcacatttgtaaaagtgaactttcttgagggttgatgacatgttttataagctacttcacacaatgtccaacacccagtgggtccacaatcaatgtatatgtctgttgttacctgtcaaaacgtatagctggggagtgacagtatggacttgagccagatcacccagtgcaaatgctggctttgctgcttactgtgtaactgacttcccagagcctcagtttctgcatttatgagattatagtgactatgccattgtagtgatgactaaatgagttaatatatgtcaagcatacaaaacagtacctggtatgtagtacaaactatgtatttgttaaatgaataccttttttttttttttagagccatggtctctctctatttcccaggctggagcacagtggcacaatcatagctcactgtagcatccaatacctgggttcaagtaatccttccacctcagcctcctgagtagctgggactacaggtgtgtgccaccacggctggctaattgtttttatatttttagagacaggatcttgctatgttgcctaggctagtcttgaactgctggtctcaagcagtcctcctgcctcagcctcctaaagtgttggatttatagatgcgagcaaacgtgcctggcctaaatgaataaatctttactgttgcctcctttgagctttattggttgggatttagtccactcattttgcaggtgagaactgagacccggagatgaatgcagtagccttgcctcaggtccacatctgagggacagactccatcctacttttcttcccatgtattctttcctgcttgtaccagttcactggggtgaccaagaaagacatttctgagttttcctcctggctggcacagtggagacatgcccagccacgtttagctagacttaccatggctgggctagaagagaggccaggagcttgtctggaggttgcacagacctgcccttggtgaatgtggatcggagcgccccggcagaggcttgaggtgctacagaggctgggggatccactgtcagaccgggtgcatcaccactgttttatgatatccagcaaattgctctgcgtctctgagcttaaggcagagagaagggcctgagcttgaagaggggatcccaggtttaaatcggagctggctacttcccagctctccactgtgtaatctggaggaagttgtttaacctctctgagcctccctttgttttgtcatgggtgtttgcagggagagcattcatgagatagtttgtatagacattttgcccttggaaggcgctcagtgattgctgtttttctctgcattttggaccgagtcccttctgcgtatccagcacctggcttcctcctgcatttcttcctcaggaatttattgagcacctatatgtgccagagcatcggaccaagtcctttgctctcctggggtttagattctagttgagcatacaggcaatgtacaaccaatacatatgttcagatcggcagctttatctgttattctgaaatccacgatttctgaaaaccgaaagtttttcataactcattttgaagctaaacttgaggctttttagagtcttcatcctcttcggtgtgatgctcagatgtctttctgcagagatattagtgcaattgatctttgggtgctttcccgccctgcatgatgggcatgtcggtgggtattttatacaaggtgacctttctacagcctgagtcatcctgaattctgaactatgtctggcccccaggattttacaggagagaacatggacctgcaatatcctatcaggtggagactgtgctaggaaggaaagcaaagctgggtgcaagtggaatcagggagggcttccctgaggaggtgacatttgagagggatgaggaagagagggccaggtggagggactcacagaggacggaagactccacttcctgactggcctgtgcttacccctcattctgcatgcacagttctgtctaaatgcgtccgttcttgcctttccaagaagtgctgatgtctcttacgtttcaagtctagctctgactgactcattctctctttaggtgcccccacaggcctcctcacctcttctcgtttgcttaagaaatctgaggtctgcgctaaagccagcctgggtgctgtaacctctcggccgaggatggtgcccagctgggcttgcatctggagttggaggaagtgggtcccgggaggggaagaggagccgtggggccttccaagggtttggtggaattgagttcctggtggggaggggctgtgttctgggtcttagagcctctcgcctgcaaagcaaagttgaacgtaacacgcgctcacatatctcgagtgccagcccggggctaggtgcttaacgcttgcttagccaaagtgccaggggctggggacacgggaatgagtgacccagaaatgagggccctcatggagctgaaagcattctgggcttggtgactttgaggaagtggcttttgaactgagacctgaaataccaaggcatgtggatatctggggaaagagtgatcctggcagtgggaacagagagtgcaaaggcccagaggcatgaatgagcttggtgtgtttgacccacagcaaggaagctggtgtggttggatggaaccaaacaagaaagaggtctaggagcagaggtcagggagggagctggggccagatacctgcattgtctcatttaacttgtgtaataaccctaccagataggagctactctcatgcaaatgttacagatgaagtaacagaggcacagagaggttcagtgacttgtccagggtcacacacctgaaggtggcagagcagggctgtgaacctagaagctccagagtcctcagtcctcaccatggtgtttaaacacaataaaatctatgcctgagtgctgaagacacacctgccatttagatcgatccacttttttacattgtcggtgtactccctgccatccatctgcccagaaatactgtgaatgtgggcagcgcaggactggttcctccaagccccattttacagacagtccaatcattttacatattattttatgaatgagcaccttcctcagtgtctcagaccaggggtccccacacagcagggccacacagcaggaggtgagtggcgggtgagtgagcgaagttttatctgtatttacagctgctccccatcgctcgcattaccacccaagctcggccacctgtcaggtcagctgaggcattagatgattctcacaggagcacaaacccttttgtgaactgcacatgtgagggatctgggttgcgtactttttcttttttttttaagacggagtttcgctcttgtcgcccagactggagtgcaatggtgcgatcttggctcactgcaatctccacctccccatgttcaagtgattctcctgcctcagcctggtggctgggattacaggcctgtgctatcacacccagctaatttttgtatttttagtagagacagggtttcactgtgttggccaggctggtgtcgaactcctggcctcaggtgatccacctgcctcagtctcccaaagtgctgggattacaggcatgagccactgcacgcggccctgggtgcgtgcttcttatgagactctaatgcctgatgatctgtcactgtctcccatcgcccttggatgggaccatctaattgcaggaaaataagctcagggctcccactgattctgcattatggtgagttgtataattatttcattatatgttaccatataataataatagaaataaagtacgtaatacatgtaatgtgcttgaatcatcccgaagccatggccccaaccacggcctctggcagtggaggctggagtccacatcacgctctgtgctgggtgcaggaatggagatgagtgggtgtcctgctcatttccctgaggaggaacctgaggctcaggctagagacttacctgggtgacacggctcctaagtggcagagtccagattcaaacccagggctacagacccagaagcctgtgcatttaaccacggttctgagctatccagcccaaagaggagagtttaaaaggattacatttcttcgttggaaatcagtgagcatagtcgcccagaaccatttttgaaaaactcagagaaggcctatgttttcctgaagcggccagggaagtattctgcctttttaaaagggaagactgaattctagggaggagaggcaccagctctctgggctgggcaggtctccaagctggcagggccacagcctcccgacggtcttgcctctagcatatttcttccagaagccacatttccacaggtgggcagctctaactgggagaaaatcttcctgagatttagccaaattagctttctttctttctttttttttttttgagatggagtctcactctgctgcccaggctggagtgcagtggcgcaatctcagctcactgcaatctctgcctcccgggttcaagtgattctcctgcctcaacctcccgagtagctgggatttcaggcacacaccaccacgcctggctaatttttgtatttttattagcaatggggtttcaccatgctggccagactggtctcaaactcctgacctagggtgatccgcccacctcagcctcccaaagtgctggtattacaggtgtgagccaccgtgcccagcccaaattagctttctaggagcttccactttctgagtctgtttctgcctcttggaggtagtgaccatctccctccaaactcggatacttctcttattcagactacacaactgtagttatttcaggtattctccctgactcaggaaattataagtttggcctgccttggcacatgagtcaactggttggagtcccacgcaaagactgtttgtttggtcaaagaggaaggagactattacctccttggatctagagcttatacttctgttgatgcaacctggaatcatactgtgtagcagccatatcccacttggagtgcatattgggcttgtcagctaaaatctccatgtttctcttctatatgagctgctgttaatccaagtttcttctattctgagagtccttgtctaaagctaagaggttccagtttttcatgctctctaataaatctacttcaggctaacatgctatcattttttctttgagtactgctttggtttctttccacaacttgttacacaatgttattcactgcccagtttagaatttcccttttggttcccattttaagacttatttaattatatgctttttaaatttttgagacagatagaattttttaagttatctttttattgttgatttctaaatttggtcatcatggttgttgtggtcaatatatgtggttcctataatatcagtggtagagaatttgctgagatctttataggattttctggtcatttttttcctcttgtgtttgaaaagaatgtatattttctgttgggtataaggttctagaaatgtatctatttgatcaagctagctattatattatctatatttgttatgtcactatgtggtttttttttttttacctgatttacctcccaccgtaatacaattacaattcaccataatagaggctttgttaagttctccttttgagtctgtcagttttttgctttatttttgaagctatgttgttaagtgcatagccatatgagtagaataagggttcaggatttttatacctttttggtgaattgttgcattttcccgagtagctgggactacagatgtgcactaccacgcctagctgatgtttgtgttttttgtagagatgggatttcgctatgtttcccgggccagtctcaaactcctaggttcaagcagtctgcccacctcagcctcccaaagtgctgggattacaggcatgagccaccgcagctggctgaattgttcctttaatcagtataaaagagccaacttccagcctgggcagcatagtgagaccccatctctaccaaaaactaaaaattagctgggtgtggtggcgtgcacctgtagttccagctacttgggagggcgaggtgggagaatcacttgagaccaggaattcaaggccgcagtgagctatgattgctccactgcactccagtctaggtgacagagtgagacattctcaaaaaaacaaaaacaaaaaaaccatccaccttctctcttctaatacttactcaagacatcattgtcatgaattctattttattgatgtcagtactcctgtactaataagccagatgagttaaaaacaacagcagccttgcaaccagccatgaagcaagtgatcttattctcatttcacagatgaggagactgaggctctgagtggactccagcctgccacggtctcccaccgagtcaggttggagcctgcacttaggcccagagttatgcttctgtgtctcagatgcttgggggtccctgcccacaaaggtggcccccatgtctgtgtgtccccacccatccaggaatggtatggggtgagacaacaggaagagctccatgggtagtccccacccactgccttccatatgccagtctgcaagcagaaaatgaacgggaagctcttacctgtgcccatgtgttgtcctggctttgggtttgcccagggggcaaagtcctggtcattgttgagacaagttcactgctgctgagggccgagcaaatggtgacaaaggccagattctaaattcctacctggttacttcctgctccgtcaccatcacctcgggcaaattctgaacctccctcagcctcattcctccatctgtgcttgcagggagaatattttactgtatcttccatagtgagctttccaggagaagcaaaaaagagtggtgtataaaaaagcaagtttatgactgcttctgtgccaggcactgtgctgtgcactccagagccaaaggcctgggttcaaatcccagcccactcactagctgtgcatccttaggcaggttcctgaccctctctgggcctcggcttctccatctctgcaatggagatgatattaattgtatctgcaatgctccttggattgggtccatggttaagtgagtgagtgaggctcaagggcttctttttttttttttagagatagggtctcactctgttgcccaggctggagtgcagtggcatgatcacagttcactgcagctgcgacctcgtgggctcaagccatcctcctgcctcatctcccttagtagctgggactacaggcatgtgccaccatgcctggcaatttttttttgtatttttggtagagacggggtcttgccatgttacccaggctggtcttgaactcctggactcaagtgatctacctgcctcagcctcccaaatggctgtgattacaggtaggagccaccgcgcccagccttcaagtgcttttttttgttagtgagactggtacaaccaatgccaaggctggtgagcaaagttgcacccagttggggaggggcgaaaccaagggcagaagggcccagggcatggcctctcatggctgaaggaagaggggacacgtggccagtcacagtgacctctgcagacctcaagaaggaggccagctaggagccatagctgtcactaagacagtcaaaatctaatcagcaccctggcgccaagaggccattgtccgctgctcaggacagtgagtgatagtcgataaccagctctctggaggtcagggccccgaggtggctgagatctgtgcctgcattaggacatttaaggggactccaagccaggatttgctagtgtttgcccagcagggagaggtggaaaattggggatgaccaaccaggcctccctgctcacagccaggggaattcatcctgtcgttcttccattcagcaaatattcatggagcacctgctgtgtgccaggcaccgtgctaggctctggggagatggctgtgagcgggatcgacactggccttgtgtgagctggccttgcagtaggggatatgagcaccaaacacataaggcccgagtgtgcaatatgccacaaagattgcagagaaataaagcaagataacaggttgcagaaagatggaggccaggctggctgctgttcctgggtaggtggcttctggacacagtttcctcaggctctgtttcctcctttgggcctctggaatcttctgaagacaggcctggcttccaggatcccggatggagttgcctgttgactgtgtggactcaaagtgtccacagtgcgccggagcgcgtggtcttccccagcaaatctgcttctcttctcggctctccatcgtgggaagggtccctgggatcctccccaccaccccagcctgaggccactttccctctgtccccgctctccccagcctgctttccccaccaagcgccaccaagtccctgctgcctcctcctcccaaacaggctcaaacctgccccctcttcgccagctctcagctcagcccgctcctctcctgcttaagagccttcactggctccccactgccctagagacagagtttgagtctactgccatggcttggcagacggtgttgtccccggcctgccaaccttccagctgccatcccagccagtccttctcccccttgctccagccaagggccccgcttgtagggtgggaagtgggccgtgccctccctcctgcctgcctattcgctgggtcttccctcattccctccctcaccccgccctctcataagaaccttcagaatctgccagacgcagtggctcacgcctgtaatcccagcactttgggaggctgaggtgggtggatcacctgaggtcaggagtttgagaccagcttggccaacatggcaaaaccctgtgtctgctaaaaatacaaaaaaattagctgggcgttgttgtgggcacctgtagtcccagctactcgggagactgaggcaggagaactacttgaacctgggagacagaggttgcagtgaactgagatcccaccattgcactccagcctggactacacagtgagactctgtctcaaaaaaaaaaaaagaaccttcagaatcataaaatgggtttaataattatacttatctcatagggagggcttaaagcacctaggccagtgtctggcaccaaggaaacactgtatacctgttaaaataagaataaaataaaatacacatgctcacttattgagtgcctgctgtatgtcgggcccctccccgagagaactctggtgcatccccgataaagaaacctgaccctctaggatgagggagggtcttccctgagcgcctgctaacagcctcctctgctaatctgaagtcctgcagcttgcccagcaggctggcttgtcacacgggctacccccacaggccgccatcttggaaaaggccaatggctgtggaatcattcttgactcctattagccttcctgttctctctcccgccccacagcatccatcctgtcagcaaatcctgtgggctctgtcttcaacacagatctagaattcagccccttctcaacctccccatctccgcagcctggtgtgagccctggcacctctctctggggccagtgtggtagcctcctcactgctacccacttctacccgtgcccccaccacccaagcaagagaggtcttttttaaagtctcatcatgtcacttctttgctcaaaacgctctggcaacttcttccttgctcaaatatttgttaaaagaagaaaggaatgaatgaatgaatggcctgtttgcatctgtaatagccagcacataggcagtacttggaaaacacccacagcaagaataaatgaggagatggctggacacggcagctcatgcctgtagcatgttggaaggccgaggcaggaggtttgcttgaggccaggagtttgaggttgcagtgagctatgattgtgccatagcactccagcttgggccacagagagagatccccatctctacaaaaatttaaaaattattcgagcatggtggtgtgtgcctgtagtcacaactacttgggaggctaaggcaggaggatcacttgagcccaggagttcaaggctgcactgagccgtgacggtgcgactgcattctagccacagaatgagaccctgtctctaaagcaaaaaagagaaggagaataagtgaggtacttgagcaagtgggtgaggatggttgagccacagcaggcccacagggaggctggtgggtgctgttggtggagggggccgatttgtgttcctggtggctgcagagccagtctgttgcctcccctccctcccactggctgggctggggacggagccaaccaggcagatcccgggccattctggggccctggcctggcctgccttactatcccagtggggccaccgacgtggctgccatttctggaaaccccagttctaggtaggttactgtcccaggaagaagtgcatgcctagcttcctgccagcccagcccagctgtcccagaagccagaggaagctctgggctggtttcttataagtagcatgactaactgattgcgccacaggagctgaggtttcaggccagtttctgtcgtggagtttcaggggtcagcggctccaaagccccagcccccacccgaggcccctgctgtccctggattcctctccacatgccacctgtcacagaggtgcttgcatcctgtctgcagaaccagcccctcctccaagccaggtttcctgcccagagggaagaagagcagaaggcctgtaccgccccctgtgcgcccccagtcctccgccccctgcccccctgccacccgctgtgggttcgggaagagtcctgctgggtcgcttccaggctgctgctggcttctgacatttcaaggcttaatcacttaatctcccccgagttttttaaaaaaagtgagtcaagtgtgttctcctggtggcgggcgtgttttcccaggcccatgcttctgctcatttcctggctgcggccttcactgactttgctgcttcctaactctgtgaccttggccttgtcacttgctgtctctgggtctcaggccaatgggtctcaggccagtggggctccccggacaaagttccagggttttggggactgcatttcaagggtcagcaaccacagtccttaaacacgtggattttggagtgacaatgcctaggttatggttctgtgaccaccagcaagtgctctagcctttttgtgcctcagtcttctcatcagtaaaatgggaatgttaatggcacctacctcatagggtcgttgggagaaccaaattgattaatatattgtaagtacttagaacagggccaggcacaaaataagtactgcatatacagttaactatggacaaaagcttgggacctggttcatgtttaaaaatgattattgaggccaggtgcggtagctcatgcctgtaatcccagcactatgggaggctgaggcaggcggatcaggaggtcaagagatcgagaccatcctggccaacatggtgaaatcccgtttctactaaaaatacaaaaattagccaggtgtgatggcgtgctcctgtagtcccagctactcaggaggctgaggcaggagaatcacttgaacccacgcggaggttgcagtgagctgatatcgtgccactgcactccagactggtgacaaagctaggctccatctcaaaaaaaaaaaaaaaaaagattgttgaatttattattgctgaatttttttttttttgagacagaactttgctctattgcccgggctggagtgcaatggtgtgatctcaactcattgcagcctctgcctcttgggttcaagcgattctcctgcctcagcctctcagcctcccgagtagctgggaatacaggcacccaccatcatgcccggctaatttttgtatttttgtagagatgtggtttcaccatgttggtgaactcctgacctcaggtgatccacctgcctcggcctcccaaagtgctgggattacaggcgtgagccactgtgcccagcctattattgctgacttttaaaccattcattcatttaatatatgtttattgtacacatgctatgcctgtgtcaggcactgtttcaggcactggggataaagcagtgagcacagcagacataaagctctgccctcatggggcttgtggtctctcagagggaggcaaacaatgcatttatagacaaatacataatttaaatttgaggttacctattatactcccaacttgaaaaactagcagtatattgaggtggctatttcacatctgatggaggaatatgtcttttgtgaaccatttatgttctttgcctatttttcttaaagggatgtttgtcttattgatttgtaagaactcttcatatattaaggccattaaccgttcataatgtagattgctacctttttctagtcttttgcttgctgatatagttgggatgtcatcctctctaaatctcttgttgaaatgtgatccccagtgttggaggtgggtcctggtgggagatgttgggtcatggggacgggtccctcatggtttggttctgtccttgccatagtgagtgagttctcacaagatctggttgtttagaagtgtgcggcacctccctcctctctcttgccctggctgtcaccatgtacgcctgctcccctcttgccttccccgtgaataaaagctccctgaggcctccccagaagccaagtagatacgggtgctatgcttcctgtacagcctgcagagccataagccaattaaacctcttttctttgtaaattactcagtctcagatatttctttatagcaatgcaaaaatggactaacacacttgagttctgtctttttcttatggcatattttaatctaggccagacacagtggctcatgcctgtaatcccagcactttggaggccaaggcgggtggatcacttgaggtcaggagtttaagaccagcctggccaacatggggaaaccctgtctctactaaaaacacaaaaaattagttgggtgtggtggcacacacctgtaattccagctactcgagaggctgagacacaagaatcgcttgaacccgggaggtggaggttgcagtgagccgacatcacgccactgctctccagcttggggaacagagcaaacctccgtctcaaaaaaaaaaaaaaaaatctagggaagttgaaaacattttgcagtcaaagctatctttgctatctgcatcttgaaataaaataaacattcatacatgtaacctcattaatctgattagagatagtgataagtactgggacagacataaacagggtgctgtgataagaagtcacagggagggcccgcattagttgaggggggtcagagaaggcttctgaagtgtttctggtgcaaatgcttggaaaagggagagctgggagtgtatcccagggctgaggccaatggaggattgcagaagagggagaggggataggaggggaatgttagagggataagcaggggaattggctcttttcttcattttcttaaattaaactttttattttcagataattaaagatttacacacagctggaagaaatcatagagaagccgggcgtggtggctcatgcctataatcccagcacttttggaggctgaggcgggcagatcacttgagatcaggagttcgagaccagcctggtcagtatgatgaaaccccatctctactaaaaatacaaaaattagttgggcatggtggcttgcctgtagtcccagctactggggaggctgaggcaggagaatcacttgagcctaggaggtagaggttgcagtgagccgagatcacaccactgcactccagcctgggcaacagagtgagactccatctcaaaaaaaaaaaaaagaaaagaaaaagaaaaagaaatcacagagagacccctgcttctatgctgttccccccgagggcaagattgtagaaatctgtaatacagtatcacagctgggatactgacactgatataagctactgaccttattgggatttcccgatttacttgtactcatttttgtgtttgcgtacgcatgtgtggtttagtgctatgcaattttatcacggcataggtttgtgtatgcagccaggatccagcacagttctatcatcacaaggatggctcaaattctccttttatagccacatccaactcccttcctgacccaccccctcagcccctggcacccactgatgtgttttccatttccatggttatgtgatttcaagtatgttacataaatggagtcacggggtgtgtactctgggaactggcttttttcactctagggcttggattttattctaagtcagagatgcttctctaatgagctgtgaaccccctggggatctgtggcccctgtgaagaagccctttaggagccagggggcattggatgtgtgagctggccctggtctcatttctgattttaaaatggctctggctgtgatgtggagaagaggcagggtgagcctcatctgcagcaactgctaactgcccacatccccccagacttctcaaaaatttgccactcccctgctcaaagaccctcaaaagctgcccactgcttagaggacaggcctttgacctgaggtcctcacttgggcttcagggattctgtgaacccctaaaatggaatgcagaattaagtgtatgcaccttgccagaaacagggtttggggtttacattagatgctcagataggtcattgatccccaaaacactaaggtctctgacttctagacagatagtagttaagaaccaaagctttggagcttgagtgctggatttaagacccatgactgtcacagctgtgcaacctcagacaaatcactcagcctttctgtgccttggtttcctcatctgaaaatggagaaaataatattacctatttcatagattattaaatgcattcttgtatgtaaaagatgtaaaagaaaacttggcatgtggtaaactctgtgtgtttaagtgaatggtgaggctgcccagtataggagggcagagctttccaggtaaagactttctaggcctcctgggattatcaggccctctctgtgttggttgttgtcatacatgacagcagtattccttccaaagggatgccaagttgtggttgcacactgcacttagatccagttgattgacagcactgttcaagtcaactgtgtccttactgatttctgcctgctggatctgtcgattaccaaaagagttgaagtaatagtaataatactattattatccaataatagtattattcagaataatcacacagatttctctatttctccttgctgctctcagtttttgccttacatattttgaccctttgttgttagttgcctacacattaaggattgttatttacatcttcttggagaattaactcccgaatcattatatagtgccacttttttttaatttaaaaattttattttttattttttgagacaggatctttctctgttgcccaggctggagttcagtggcatgaacacagcccactgcagccttaacctcctgggctcaagtgatcctcctgcctcagccttcccagtagctggggctacaggcatgtgccacctctaataacttttctttaatttacaatagtattagtttgttctcatgctgctctaaggacatgcccaagactgggtaatttataaaggaaagaggtttgactcacagttctgcagggctggggaggcttcaggaaacatattatcatggcggaaagggaagcaaagacatcctttttcacatgacgtcaggaaggagacacgccaaggaaagggggggaaagccctttataaaaccatcagatctcaggagaactcactcactgtcacgaaaacagcatgggggaactgctcccgtaatctaatcacctcccatgaggtccttcccccaacacgtggggattacaattcatattacaattcaaggtgagatttgggtggggacacagagccaggccatatcacattattattaattagtgccttctctctcatttttttgattcgcctaggtagagatttatcaatttcattgatcttttcgaagaaccaatttttgatttcattgattttcctcgattaattttctatttttaatttcattgatgtatgctccaattttgtatttttttcttctgcttgttttagacatatattgcttttctgtttctagtactctagatggaaacagattattgattttagaactttctttctaagatatacattcaataatataaatttccctctaagcactgcttttgctacgtctcaccgattttgataggctatattttcatttttatttagttaaaaatatttttagtttctcttgtgatttcttctttgatccatatgttattcggaagtgtgtttttaaatctccaaatacttggaaattttctggctttcttcctgctagtgacttctaatttgaatccattgtggcctaagagcatatgttgtgtgatttctaatgttttcaatctcttaaggttgttttatgtcccagaatgtggtctgtcttggtgattattccacatgaacctgaggagaatgtatattctgctgttgtgagatgtaacagtctataaatggatacctctgttaatatgacctcgggtgactctttttttttttttaagttgctgcttcaccattggcttttggagattttgcagatgataaaatcctcaggacttttctgctgctcagtcagattttcttatcctacacttgcggaactgttgtttgaaattgacaagcaggatggccaactctgcccggctcattttatttcatcttattgggttcagcctgcagttttaggagatgagattgaaggaactggtatactggaatccacggtgggctcggaagcaaacaaacgtgggttctgattctagctctgaccttttaccagctgtgggacctggtggatgggttatttggcctctctggcctcagtttccttagctagacagttgggataacagcctacctttcaggattcttgtgtggattcggtgaggtaatacttctaaagcacctagcaaagtgtcaagtacaaggtaagtgctgggcaagcagggattttgttcacacaaagccttgactcaacataaggaggctgtttccaatggctctgcttaactgagacctgagagtgattgctccatctgtggtggcatatgggctgaaactgcctagcagcatgtcagaagtggggtgttcgcaggacatccttgtccaggttggcagttgagcttggcaggtgaccgctgagaccccttccaactccaccacttcaggaatctcttttttttttttctgagacagtcttgctctgtcacccaggctggagtgcagtggctcaatcttggctcactgcaagctccgcctcccgggttcaagccattctcctgcctcagcctcctgagtagctgggactacaggcacccgccaccacgcccagctaattttttgtatttttagtagagactgagtttcaccatgttagccaggatggtctcgatctcctgacctcatgatctgcctgcctcagcctcccgaagtgctgggattacaggcgtgagccaccgcgcccagccaggagtctcttgatttagaatgggaacttgaaatcatctcctcttgccgtgtacttgaacgcgataacctggcacttatctttttaattcttttactcacctgcatctttgttctttttgcctctgatgttcagggaagcctcttctcccactgcatctgtgatctctcctgaaatgtagcacccacacatccctgagaagctagctgcggcacgtgtgtgtgcacatgggcatgcgtaagtctgggcgtgccttccacaacagattaaggatatcaatcctgacgccatgctgggatgttttagtgaatcttgacagggttgttgactcccctgtttccctggaggactttaccatgaaagtcttctgctgtgtgactttggacaggcgtcttggcctctctgtttctctttattcataattgaggctaaagtgacacctttgccatctctgccacaggatactgtgggcatctgtgtagttgtgagagtaaaagttactaggacttgccaaggaaatcagtgtcaggaacccaccaccccattcccgcccctcctttacttgagactttacttttgtaactctcagcaaggaagacgagttcaacctttgagccgatacagagccttcccctagagagagctttgcagcctcatgctgagccagttaggggccccacatcatgtccaaaatgaatttaatcaacaaacagttactgaacacgtcctatgtgccaggcaccgttgtagatactggggactcggcagtgaatgggacagaccctggctgtcaggatgacagacaatttaaaaagggacagatgtcccacgctggcttgtcctttgcacccctggcacttcgtgaggagcagcggccagttcttgtatatagactggcccttggtgtacatttgtctgacgtttccaaatgatgtatgcatccttggcaggaacccaggaaaagacatgctgtgctcttctcaggaattgtttcagggaggtgatgttggggatttgtcccattcttgttggtgagtttagatcacttggtgaaggtggtgtctgccaggtttctgtattgtaacgttcattgatatgtattttcactcattagttaatatacaatacaagttttgcagggagttactttgaggctgtcatagtaaaatagagctttcccttcttccctgtttatttatttattagagatgaagtctgactctgttgcccaggctggagtgcggtggtgcgatcatagctcactgcagcctccaactcccaggctcaagccatcctcctgcctcagcctcccaagaagttgggattacaggcacacaccacactgcacccggcctcctccatttattgatctatgtcaatacagactcctggactttaactccaagggttacaagtggataccataatattcattttgatgctcaaaatgtgcctgttttgtccaggtcaggctagctgctgagtacttccttattgtctggcacaagaattccaagcctatctggtacttttccttctcctggaatcagctatttctctaaggcgctctggttcctttcagtacagaatagtatttagaagccaagacctggtcactaggaaggcatgataatcaaatgtgatgaatgatcttggattgcatcctggacgtataaagggcattattgaggcaattagcaaaacataaataggggccgtggatgagatagtggtgatgcatcactgttaatttcctgatttcgatggttgtattgtgattatgcagagaatgtccttgtttttctgacatgcacactgaggtattggggtaatttattcattatggagatatgtgagtgtgtttatgtgtctcatacacagatgtttcttaatgatggggttttgtccccataaactcatcataagttgaaaatattattaagtcagaaatgtacttcatactcctaacctactgaacgttatagcttaacttagctcggaacacttgcattagcctacagttgggcaaagtcatctaacacaaagcctgttttgttataaagtgttgaatagcttgtgtagtttatggaatactgagagtgaaaaacagaatggtcgtatgggtgctccaagtactgcttctactgaacacccattgattttgcaccatcataaattaggggaagagaggaaaagcacgcgtaagggaggaagggccgggtacagtggctcacgcctgtaatcccagcactttgggagaccaaggtgggcagatcgcttgaactcaggagttcgagaccagcctgggcaacataatgagaccccatctctatcaaaaatacaaacacttagccaggcctcatggtgcacatctgtggtcccagctactcaggaggctgaggtgggaagattgcttgagcccgggaggtggaggttgcagggagtcgaggttgtgccactacactctagcttgggtaacagagtaaactctgtctcaaaaaaaaaaaaaaaaaaaaggaaagggaggaagggaaggtccctccaggtggaggggacagagagagaaaaggcatggaggtgggactgagtttggtgtgtttaagaagtgcaggtggcagccgggctcagtggctcacgcctgtaatcccagcactttgggaggctgaggcaggcggatcacctggggtcgggagttcaagaccagcctgaccagcatggagaaaccctgtctctactaaaaatacaaaattagttgggtgtgtggcccatgcctgtaatcccagctactcagaaggctgaggcaggagaatcgcttgaacctgggaggcggaggttgcagtgagccgagatcgcgccattgcactccagcctgggcaacaagagtgaaactctgtctcaaaaaaataaaatgaaataaaataaaataaaatgttaaaaaacaaaaaagaagaagtgtaggtggcaggcgtaggatgcatgggtgctgggcccaggtggagagggaggtggagccctgctggggggagggccttcaagtgtgcccaagtcactcggagagcatccagcaggggcctagagctcctgaggtggccttaagttcttatgatgccccagacactgcacacagtgcttcagaggcaacacctccgttcctgtttgctgcaagcctgtgaggttagccttggaaccattcctgtgccacagaaacacagccagagcaccaaaagagaaatgctgttaatggcaaagaactactcactagcagaacaaagcctgggagctgcttttttgaataagatcccaggagagagacccaagcgggaagtgaaggatgggcacttgaaatgttgcagggaatataacccagcgcgtgctgttttctttttttctttttcttttttttttttttctctttttgcaacagggtctcactgtgtcgcccagcctagagtgcagtggtgccatctcggctcaccacaacctccacctcctaggctcaagcgattttcctgcctcagcctcctgagtagctgggactacaggcacgcgccactaccgcccggctaatttttttttgtatttttggtagagacaggtttcaccatgttggccaggctggttgtgaactcctaaagtgctgggattacagatgtgagccaccacgcccggccccagtacacactttttggagggtatttgacgttatgtaccaggagtgctggggaaaagtacacacttgactccgtgagattctgcttcttagcatttttttcttttcctttttttttttttttgagacagagtctcactctgtcacccaggctggagtgcaatggcatggtctcggctcgctgcaacctccgcctcctgggttcaagcaattctcccacctcggcctcccgtgtagctgggactacaggtgtgcgccaccacacttggctaatttttgtatttttagtagagacagggtttcactatgttggccaggctggtctcaaactcctgacctcgtgatccgcccacctcagcctcccaaactttctgttttcttttcttttcttttttttttatgacagggtcttgctggagtgcagtggtacggtcttggcttactgcagccttgacatcccaggctcaaacgatcttcccacctcaacctcccaagtagctaggattacaggcatgcaccaccgtgcctggctgattttgtttaatttttgtagagataaggtctcactctgttgcccaggctggtcttgaactcctgggctcaagccatcctccctccttagcctcccaaagtgctgggattacaggtgtgagccaccacacctggcttctgaacatttttttcttaatgataatagctggtgctatgggctgaatgtgttcccccaaattcatatgttgaaacttcatcccattgtagtcatattaagagatggggcctttggagaagtgattaagtcatgagggctctgtggttggtcatgaatggggttagtgccctcatgaacgggctgaaggaagtgtgtttgccccttctgtcatatgaagacacagcagcatggcatcgtccgtgaggaatgggctctcaccagatctggatctcctagtgtcgtgatcttggacttcccagtctccagaactatgcattagaaaatttatttatttattatttatttattttgagatggagtcttgctcttgtcgcccaggctggagtacaatgacgcgatctcgactcactgcaacctctgcctaccccagttcaagcgattctcctgcctcagcctccagagtagctggaattacaggtgcccgccaccacactcggctagtttttgtatttttagtacagacggggtttctccatgttggccaggctgatctcgaactcctgacctcaggtgatccacccaccttggctccccaaagtgctggaaatacaggtgtgagctactgtgtctggcctgaataataaaatttaaaacaatttttcaaaaattcaccatgaggtctcactatattccctaggctggtctcaaacccctggactccaagtgatccaccccaccttcccgagtagctgggactagagatgcacaccattgcacccaatagagcaatacgtttctgttctttgtaaattacctgctctaaggtatttttgttatagcagcctatatggactaagctgacttgtaacgttacttgagactttaaagtgttccggtcactgttggagggctctgtctgtgttagctcatttaatccccacaacacctcaatcagatggggctattcttagtcccactttatagataaggaaactgaggcatggaagcacagcttgctcaaggttcacatctagtcagtgacagagcaggtatttaaacctcaggaaataatcagagaaacatgtgtagagggttgtccaaggaaggccacatccagaagcatctcccaggacagttgttgtgtagctcaccctctggactttgtgggtctgggtgttgtttcatgattatagagagagctctgtgaacgtggaggacctgttgtcggcagagacacaaatggccagggcatggctgggcagccgcagtggctcaggcctgtaatcccagcacttcgagaagaccagaggggcagatcatgaggtcagaagttcaagaccagcctggccaacatggtgaaaccccgtctctactaaaaatacaaaaattagccaggtgtggtggtgggcacctgtaatcccagctactcgggaggctgaggcagaagaatcgcttgaacccgggaggtggaggttgcagtgagctgagattgcaccactgcactccagccttggagacagagcgagactctgtctcggaaaaacaaacaaacaagcaaacaaacaaacaaataaatggccagggcaggggagggttgcatattgaataagatgagctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgccagggtcctgcagtatgtcacctggccttatggagattacactgcagtgggaggggacagccaatgacaagtggccctgattatcagtaaattctaaagattgttagaaagtgatgggagccgggtgcagtggctcacacctgtaatcccagcacttcaggaggccgaggcaggaggatcgcttgagcccaggagttcgaggtcagcttgggcaacatagggagaccttgtctctacaaataataaaatattagccaggtgtggcagtgcacgcctgtagccccagctactcaggaggccgaggtgggaggatcccttgaactcaggaggtcaaggctgcagtgaactgtgatcgcgccactccactccagcctgcgtgagaaagtgagaccctgtcaaaaaaaaagagaaggtgatggggaaagaacacagaacagcataagagggggttggggaagctgggtggagtgggggggattgcagttgaaagtagggaagtcagggaaggcctcattgagctgacttggaggaagcgggaaccgtgcagatgtctggggaaggctcattcttggcagagaggccctgcactgagcctggcgggagggttgagcacaggagggaatgtggtggaggagagtgagcagcaggagggagcagtgaaggtcagcaaggtgacagagtggctgaatcaaaaaagaccttgcagtgtttgagcagaggatccatatcatccattatgttccaaaggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaaggtactttggggaggggcgggaatcaggagttagttttggacatgagataagcttggaatatttatttgctatctaagacagctccttaacatggtaagcccttatgcaagttgttgtcagctgagatgggcgtggcactgagcatgggagcatggaggcgcctgagtggtctcatgctcaggtggtttagcaaactcagtgtacatcctgccaattccagtcctgccatggccactgacaagctaggagggcgctgaaaggagaaggaccccgatgtctcctccagcccatccatctcctctctcccattggccaaacccaaccggaaactaaaggccaagggtacccggtgatgaagactgtggtatcagcctcctgagcacagagagggcagaaaggggtggagacaaagaggggcgcagatagtgggcaaatggggaagtggcacttcccctagctcgagggcagaggcttggtgtgatggaatggcactccttaaactgctacatattttccctttaatttggccaagaacaagttgtcaagtttgtgtgagataaaggtgcacttggttcgttcttgtctaatggcccccgcacccatgggtatttcttcagcttccacagtcatcccgacactagctgggaagctccagcagccctggtcctggccccagctctgtgggcgctggccctcaactttgcctgcactgtgcttttgtgctattccccttggtcctgtttgggtgcaagtccccctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtttgccccaagagtgagctgggtccaggtggtgcgctggagtgcaggatgctgagtatggtttgctgctgtttatatggtgttagaggggaggtcccatctccagggacatgttatgtaagatacagtggagcgcatggtgggagtgttggtccacgtggcacatggatacggctggaatactggactagaccagcagttctcacactttttggtctcaggaccctttttcacacttaaaaatgagtgaggacccaaagggctttggtgtaggtaacacatcattctatgtttacctaattagaacttgcaatgaagaaatggtgtaatttttaaaaaattaaaacaattaaaaattttttttcttactgaaatggaggtctcactgtgttgcccaggctgctctcaaactcctgggctccagtgatcctcctgcctccgcctcccaaagtgctgggattacaagcgtgagccgctgtatccggcccaaaatggagaaattttaagtcccaacaacatgcaagcccgcattcaacaaatcttcagatcaattacatgatcacaggtcatgtagcctctagaaaattccactgtacgccagtgagagagagtgaaaaggcaaataacgtccctgtattatgatgaaaagagttttacctggtgggcccagaccacactttgagaaccactggactagacccttgattgaggagtacggtgttgagagtggagtcctctgtgatggtggatggaccaggacacatggcataggagtcaggtggttccctgggctactccatggtgcacaggatgcttcgttacactggtgcccaggacataatcacgtacacaagacacacagttacggggcagactggggatatacggcacaccagcatgcagcgttcaccagtaaaggtggtattccatgattattctaaggtagatgggctgtgctttgtttccattggcttagtccagggattggcaaactatggcccgtgagccaaatccggcccactgcttgtttttgtaaataaagttttattggaacacactggctgctgtagttgtaacagaaactgcatggccctcctttatgttttttgtttgtttgtttgtttgtttgttttctttgagacagagtttcgctcttgttgcccaggctggagtgcagtggcacaatctcggctcactgcaacctctgcctcccgggttcaagcgattctcctgtctcagcctcccgagtagttgggattaatggtgcctgccaccacacccggctaatttttcgtatttttagtagagaccggttttcatcatgttggccaagctggtctcgaactcctgaactcaggtgatccacccgcctcagcgtcccaaagtgctgggattacaggcatgagccactgagcccggcctcctcctttatcttaattgaaataattcagaaatggaaagtcaaatactgcatgttctcacttataagtaagagttaaataatgtgtacacatgggcattattccatgtaccatggaataacagacattgaagacttgggagggtgggagaggggtgaaggaagagaagttacttaatgggcatagtgtacaccatttgggtgacggacccaccagaaccccagacttcaccactaggcagcatatccagtgagaacagatctgaggcttgccatcaaaattgcacttgtaaggccgggcactgtggtggctcgcggctgtaatcccagccctttgggaggccgaggtgggcagatcacttgaggtcaggagttcgagaccggcctggccaacatggtgaagctccatctctactaaaaatacaacaattaactgggtgtagtggcgcacacctgtaatcccagctactagggaggctgaggcgggagaattgcttgagcccaggaggtggaggttgcagtgagccgagatcacatcactgtactctagcctgggtgacagtgagactttgtctcaggaaaaaaaaacaaaaacaaaaaacaaaaaactcgtaccccctaaatttatacaaataaccaaaaaaaaaaaaaaaaaaggaaattgtgtggcctttgaagtccaaaatattaactatctggcctgttacagaaaaagtttgcagacccctggcctagcccgtgagatgtgggttggctgttaaggtggaacattggaattatcttacgatggccaaactgtgcgatgcagagcttatgttgttctaaattaattagtgccaccggttcttccctttcatgggctttcaggaacaagctaagtcccaggaccagggccggcagctaggcaggtgtgaggagcatccttggtgcatgtggtaagaggctgtggccagcaagagaggcaaccctagtcggctgccccagcacaccctggccgctcccaagcccccagatctgtcctcacatccgtgatcgggaagctggaagagtctgatgcggttcctggaggcatgtcccggacacagctgtggggcccagccagcctacaggtgaccagcctaacccagcccctgtgtctgcagagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctgcctgggctgagggtggggtgggcaggggaggaggtggggtcatagcaacagcaggaggaagccgcctgtattttcccaaatctgatgggattcctgcccctgcctgggcctcagtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacattccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatggtaaaaccccgtctctactaaaaatacaaaaaattagtctggcatggtggcaggcgcctgtaatcccagctacttgggaggctgaggtaggagaatggcttgaacccaggagaaggaggttgcagtgagcttagatcatgctactgccctccagcctgggcgacagagtgagattacgtctcaaaaaaataaaaataaataaaaataaaaagattttttaaaaggctgaagtttgggttactttggctcatacactttgccttcactgtagaaaggtggttagtaaagaccaggcgcggtggctcatgcctggaatcccagcactttgggagcccagcgcaggcagatcacttgagccctgggctattgaggctgcagtgagctgggattgtgccactgcactccagcctgggcaacagagtgggaccctgtctcaaaaaagaagaaaaaaagggtaattaataaacactaaagttctatgtagaattttagcaacattattgttattataatcttctttgctatggctctgaatctgtgtggtgctccagaagtatgctatggaggttttgtcgaccaaaaatctgggtggtggctgtggtttgtaggccggggctgggctgggtgatgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtgggtgaccagcagaggcccagtccctggagccaggagcctgggaggcaagccctggggctggatagcaaatcccaggagctagagacctggcttctcacctggctctgcactaggcaagtccctttgcttcctggccccccacccctcacatcagagaaggggagttatctctgcatgccgctcctcctctgtaaaggtagggctgtgggccacatctgtgtttcccagtttgggggacacaagtgatcgtaggtggcacattgacagctcacttgaataaccctattattgaagagaataatactgactcaagagacagtgacccgtgtcagttcccttttgaggccaacgggttaaggaggaagtccccatacagctgactcgtttactaattcctcttaatgaagagagcagaggccacaccccaggcttagactttcccaagaaaacaagatcagtttgttggttgttccccatggaagctggtcctgacattcccttcacagtagtgttggtggagtttttgttgttgtttgttttgagacagagtctcactctgtcacccagggtggaacacagtggcgtgatcttggctcactgcaacctccgcctcctgggttctagcgattctcctgcctcagcctcctgagcagccgggactacaggcacctgccaccgtgcccagctaatttttgtatatttagtagagatggggtttcactgcgttggccaggctggtctcaaactcctgacctcagatgatccactcgccttggcctcccaaagtgctgggattacaggtgtgagccaccgcacctggccagtggagttccttcttaagtacatgtattgacatctttaaaaagggcgagaggatttacaggaaactatcaggtcagtaatggcaggggccgtccacagtgggtggctgagtccccctatttttctgctggtgtgcagggaggtcatttcctgccacccatgtttccccaccctgaatccaccttcctcacattcccattggagggacaatctctggacatatgggacctggggtcccacagggctgcaatccaatgcctgctgtgccactcgccagctgtgtgatgttgggcatatcccataacctctttgtgcctcagtttcctcatctgtaacacaggagtgacaagagcacccgcccacagggctatgacagtacaaggtgtgtgatacagatgagctcccctgtttggcccacatgtgtcctaaaagccatgtgccctttctcttgagtgccccaggccacagagatccccatctgcccgctgtcccacacactggtctgtcatttgttccttgaggtttgtgagggccggctctgtgcatcccaggggcccaggctgggcctggttggctctcagggagcaggcacccgccaccttaagctcccatgctggtgtctgtcactgcttcctctcaatctggccaagccaggggtgtcgatttatatctctcaggtctggtttcccctttggcactgggccaggtatggggaaagagcaggaatggggcagttggctcacacagcagaggctcagaaagcggggggcatggggggaaggagtgcacagatgctagagagtggggcaagttttgtttggtcaataaatctccttctcatgccccaggcctgtgcaagacctacagagagtcccaaggatgggctggggggaagagaaaggtaccaccttcagagtccaaagatatgttatttaatattttcatatttctagatctgccttcaggcatggctggatccagcttctaggaacctgtccagctctgcgccctgctttattctgtactggcttcgtttttaggcaggctcttccctcatgtagtggcagatatgcctactagttgctccaggcctacatcccaaagccacagtgggaaaagggttttttttcttgacggttctaataagagtcctaaggctgctgctcagtggcctggcttcgatgctgtgccagcctctgaaccaatcactggctgtgggtggagagagggtgctggtggagggccctgcttgtccagggaggagtcacatacctgcctctagggctgcaggtgggctcagctccatccaaaccagatgaactgaaaataaggcaggagtggcttccccaggggaaactggggaagaggaagcaggactgtgctggctaaaatgccagccaggtttaagacgtggcaccagatgccagtcatgggattggattggtcagcatgcctgggctatggcttaggggtatgttggtgctcagggatgccacaggcctccagataccaggtctgaggcagaagaatgaagtccagcttctcttgtgggtggaacagtggcaactgagataccccatctctcccttcccaagaacagagctgaacataaagaatttagtgattggccagagcttggccacatgctcccctctgatgaatgataggccaggtgatgggattggcacaattggcttagactaatgagggttggccctggagttgcaggcagtggagttctgtcctaagcagtgggcacctaaacccgatggcataaaagctgggcgggtgtccacctgcatctgccacagcactataggcaccaactgtggctcatactgagtgggataaattccagaaagaaacattaggaacttactatagaattttggggctagagctactcattcattcccctagataatttctaggcaaggttccatagtggagggggagttttggcttgggcattgaaggatgcataggagttttctagatggggaaagaagggaacggtagaccaggcagagggaactgcatgataaaaggtttatgggtgtgaaaattcatggaatgtttgaggattatggggttgggggatgtgggaatatgtgtagcgataaagcaccaaacaaagccaaaagtttagttagagccctgaatgcctgcctcataatggtttccatattttatatgcctactatgtgccaggcacattgctcagggtcacacagctggaaatggcagggctgagtttttgttgttgttgttgttgttgagacagagtctcactctatcacccaggctggaatgcaggggcgtgatcatggctcactgcatccttgacttcctgggatcaggtgattctcccacctctgcctcccaggtagctgggactacaggcacaggccaccacgccaggctaattttttgtatttttagtagcgacagggtctcgccatgttgtccgggctggtctggatctcctggcttcaagtgatccccctggctcagcctcccaaggtgctgggattacaggcttgagccaccgcatccagcccagatctgagatttgcacccagtatttgaactcccaagcctgtgctctttttcctcccatggacatttctctcagagatggtctcccaaacacctgtccttcttgttaaaaaacagacaaaccgcaagtagttctttggaagctcagatttctcttttgtttcttagtaaaacatttcccagttcccagctcccttccagggtgtaagatttcttcggtaacttacatctagctgttgcttcttgtttgctcatgtttagaaagaaagacaaaagagagtgagaattttctctcccttccccagtctccccacaactcacaccccaccctcagctccctctgtaataggaaaatctctgaactctctgtagttgctccagcaatcttttggaactttgcttctttcttgtgaaaaaacctccccttggctcactttgcaccaggtttccccaaatgtgcttccaaccacaagcagaaatggagctgccagtaaccaggaagaaactgccgggggctgaggaagaggagagggaggtgcatagccctggatctcgcagggagaggggtgacaggatgagaactcaggttgctcacttgccatcagggtcagtcatgaatatagcgttcatgtatcactttttaaagcttttttggagggtaaaagtaatagttacacaaaataaaaatacaaatggtacaaaaggacttagaatggaaacatgtttctctcccgactccagcctcctgtttttcttcccagagactgaccactgctgtctgtctcttgccagaagggaaagggaggcaaggttagggcaggcagagggcatgtgcatcctttagagagagcttatgtctatacaagcaaatgtgtgtgttcagtcatcgctgtcttagttttctattgctgcataataatggtactaccagcttcacagctttaaacaacacccatttattatctcatagtttctgtggttgggagtctggacatagcttagccaggttctctgctttagagtctcgtgaggctataatcaaggtgtgggatggggctgcagtttcatctgaggctcaattggggaagggtcacttctaagctcatacaatattggtgacattcagtccctggcaggctgttgaactgagagcctcagtttcgtgctggctgttggttgtagttaaccctgaattccttcccatgtgccctttgcaaagccatcaaggcagagagacttgcctagcaagtaggatattacagtcttctgtaatataatcacatccatgaaatcctctatatatcccatcacctttaccatattctgtgggttagaaacaagtagcaggtcctgcccacactcgagaagaccagatgacacaaagatgtgattcaaagtggggatcatcggggccatcttaggtttgtctgcagtgatcactgtgccatctctctctctctctttttttttttttttttttccgagacgaagtcgtcactctgtcacccaggctggagtgcagtggcatgatctcagcttaccacaatctctgcctcccaggttcaaatgattcttctgcctcagcctcctgagtagctgggattacaggtgcccgccaccacacccagctaatttttgtatttttagtagagacagagtttcaccatgttggccaggctggtcttgaactcctcacctcaagtgatccacccacttcggcctcccaaagtgctgggattacaggcatgagccaccatgcccagccccatctctctttaaaaaacaaacaaacaaacaaaaaacataaaaagaagcagagaacacatacacatctgcatcttcccttgtttacttaacaatagatcttggaagtcacttctcagtagaggctaggttgggcagagcattggattctaggccagtgagtttggacttgaccatggagacactaggaagcccatgaaggacagagagagatgcctcgaccctgccagtcctttagaaagatcacccagtgctttttgtataccaaaccctatttgaaatacttacgtatattaacccatttccttatcaccacaaccctgcgggaagggagataggcacttttattatcttcattttgcagatgaggacattgaggtccagagaggttatgtcacttacttaaggtcacacagccaggaagtggtagtagggactcttacccttgttttacagatgagattgaattatctcacgaaaactcagaaaggttaaacaacttgcctaagtaacatacagctaattagtcgaggagcctgacgcatgttgctgtagcctggtcacagttacagaggtggcaagcaatggcctgaacaggacgaacaaccaaatacccaggctggtggctcttaaacatggtggggtcagctaacgacagcaaccagggtgggcactggtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggcttttatatcataggatgcctctaatggcaatcctgccattagatacacctgccgtggtgtatctgccaggtaggcaggctaggctgcagtaacaaacaagcccacaatttccatggcttaacactatgggaatatatttcttgctcacgtaacaagctaacgtgaatgttgctggtttgtaggtggtttccctccctgtagaaatctggggagtgaggttctttccatcttgtggtgccgtcattctccaggacaaagattcttacctacttttgtgtcctggtttcctttggcagcctggtgaagcctatggacctcatttcagaatatttttaaatacataaaatcccagcctgggcaatatagtgaaacccccatctgtacaaaaattagccaggcatggtggcatgcacctgtagtcccaggtactgggaaggctgaggtgggaggatcacttgagcccaggagtttgaggctgcagtgagccgtgatcgtaccactttactcccacctgggtgacagagcaagagcccatctctaaaaataaataaatacaatgaaataaaataaaataaatagaactacagaggaaactaattgtattgaaatgcagttataaaacatttaaacacatttttaatctagagatatatgtgcttctttattaagatctataaataataagttctaggggtagctcgcataaatactgtaatttcaaagtagataagcataaataatactttatgatactgaaattgtgatgtgatatgagaatagctgtgagttttgttttgctggggacaggatcagtgatgctgtcattactggggtctcttccctccattctttttttaaaattgtattttattttatttttaaaattttaaaataaatagagacagggtatcactatgttgcccaggctgcttttgacctcctgggctccagtgatcttcccatcttggcttcccaaagtgctgggattacaagtgggagccagtgttcctggccccttcctccattcttaatggaaggagatgctaggtgtgagaggttagggaaagtaaagatgtaatttctttcccatccaagttctcagacccctgaattctacctgcagccatgttggtccatcaaccccaagtgaagaatccctgctctagggccccaccattgtctgtatccagccagcagaagaggcgtgattatggagatcacatctgcttcttgaaagcagacagcccggaagtgggccgcatcacttcctctcaaattctattggtgaaaatggtcacatgactacacatagccacaaaggaggctgggaactttctcacttggaacctacatcccagaaacaactcttttcagtgaggtatcccacaggtctttcgcagtagaaatattgattatctcacataaaatgaagtcttacaaatggacctactgggttttgtacagcagccaagtgatatctcttcccttctgctgtcttcccttctgccatccttcacatggtggcattgtatccttagacttgccacccatgccctcaggttggccgttgcacactgtcttacataaagcaggaaggaaaggaaaggctgctacgagagagtgtaccttgtgcatctcttttttaatcaggaagcaaacatctttctagaagcttccctagcaaaattccccttacatctcattggccaagactgttacatgttacatggttactgttattacttgctcattgcaaggaagactgggaactcaaatgcctggaaaaaggaacaggataatcgtgattggctcaagccttagggtgggcatggctccctgacaagggagagaggaaaaagctgttgagtgaagaagactgcttcagtttccccatctgtataatgggaggagtaagggctgtcgtaaaaactcaatgaaagaagattcttcaacgtggtaggtgcagtggcagctggcagtaccctgaccctgccaccgcacagccctctcagcattgctcatcctgcactgtggatatcagttgagccacgtgtctcctgccctgggctgtgagctccataggcagggtctccatggctgtatctccagaacccagcacagaaccaggtgcttgggaaagttttgaattgattctcatctgccattggcatggggaagggaactagcttgtatgaaacagataacaatgtatgggaccctcattcattatttcagcaaatatttgctgagttcctcctacatggctagccctgtgctagacactggggaatcggcgatgaacaaagcagatagaaatccccactcttgtggagctgacattctggagggagagacaaaaagcaaacatataaagaaagaaagaaatcacatggatctggatgacagtgagtgctgggaagaaaataaaagcagaggaaggggatggagcgatgggcagggggcaacggtagggagggtgtcggggaaaactttttggagaatgtgacgatgaaagtgaacaaggagaagtcaaccgtgttgagatgatggcagctaatgatgtggacaggccactctgttctgagtgcattatctattgattcatcatgtcatcctcgcaacagccctgcacgatcaattctgtcattaaccccatagtacagatgaggatgcggaggcacagagaagataagggacttgtcctgtgtcacacagcaaggagccatccggctcctaagttggtgcatttgacttctgtgcttccggaaagaaagagcagcaagtttaagatctggaggtggcactgagctttggaggagcagggggcaatgaggtggccggtgtgacgaggactcaatgtgcaagagggagagtggtggggagatgaggtggaggggtggtcggcggtcagatcgtggagggtctcggacgagggtcctgaccctgggtctccagtcctgggaagtggagcccaggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtggcagggacttgcccctctagtctgcccctttgcagtcctctcagtcaataatacgcatttactgagcagctactacacaccttgagagtagagctgagaacatatcgacaaggaccccacttttttctttttttttttttttttttttttttgagacggagtctcactctgtcacccaggctggagtatagtggcacaatcttgcctaacagtaacctccgcctcccgggttcaagcaattcttctgcctcagcctccagagtagctgggattacaggcgcatgccactatgcccggctaattttttgtatttttggtagagatggggtttcaccatgttggtcaggctggtctcgaactcctgacctcatgatctgcctgcctcagcctcccaaagtgctgggattacaggtgtgagccactgcacccaaccaggactccacatttctaaaaccggcatcctactggggagactgaaaatacatatcaatcacaaacaggtggttttccatagtgacccactctctgaatgcactagaccagggtgcaggccagagatcttctggggtgctttttgcaagggggaccaggataaggctctccaaggagggaaaatttgaggggggccctgactggggagaatgagctggccagggataagcaagatggagtcatcccacatccccttacaacgctgggtgcctgggcaactgggggcatctgggggcatgtggtaggagccagaggaatttgcgacgattgccctgatggagtcaggagacctgggtttgaatcctggccttggagcttggtagctggcggccgacaagttgctgaaacccctgagcctggggttcctgctttgcagagtgacagtgatggtgagaacatatttcatcagccagaagaggccaaatcacagtaaaggctgagggaggagatgagtggcgagtggctgggaggtggtggaaggagcctcgtttccagagagctcttgccagcccttggaatcatggtgtctcagagcctcagtcctcccatctctgaaatgggactagcaagctcaacctcactaagtcaggattagaggtggctaaggattattaacatgattgatgaaagtgcccactcttggcccagcacacactaggtaggcagggaatgcaaattcccctccatatcttgtcactgatgcctccgagcaaccttggactgatcgccttgctctgagcctcagtttccccatcacctgtacctcttcccactccccatcactatatcccagcatgccagcctctttgctgttctttgtctttggtttcttgttttgttctgttttttagacagggtctcactctgttagccaggctgaagtgcagtggcgcggttacggctcactgcagcctccaattcctgggctaaagagatcctcccatttcaacttccagagcagctgggacaacaggcgcttgccaccacacctggctaattttcttattttaatttaatttaattttattttttgggacagagtggagtctcaaaaaccaagctggagtgcagtggtgcgatctcgactcactgcaatctctgcctcccgggttcaagcgattctcctgccttagcctcccgactagctgggattacaggcgtgtgccacgacacccagctaatttttgtatttttagtagagatggggtttcaccatgttggccaggatggtcttgaactcctgacctcaagtgatccacccacctcgttctcccaaggtgctgggtacaggcatgagccactgtgcctggccaattttcttacattttgtagagactggctgtcacttatgtagcccaggctgatcttgaacttctacccctttatctttattcatggcacttattaccatgaatgaatgacctcatataagcatttctttcgtttttttttttttttctttgagatggagtctcatgttgtcccccaggctggagtgcagtggcgcgatctcagctcactgcaacctccgccttccgggttcaagcgattctcctgcctcagcctcctgagtagctgggattgcaggcgcctgccaccatgcctggctaagttttgcatttttagtagagacggtgtttcaccatattggccaggctggtctcgaacttctgacctcaggtgatacacctgccttggcctcccaaagtgctgggattacaggcgtgagccaccatgcctggcctcatataagcatttctgtctccatttatcatccatctttccctcttgaaggtcagtttcaccaaggcaggcatctttgtctcgttcactgttgtggcctcagggccaggcacagtgagtcaaacatagaaggtgctcaataaatatgtgtttatttattgaaaccatgggcagaggctaattcagaagcggtctgaggaccttacctcccagtgatgatgcaccatggccccaggcaggccaggaagagagaagggttgtgtttctccgtaggtcccccagcttcccaggccatcccaggccattccctggtcatttgccctcagctgctctgaaaaagggattgttgaggggaacctagaatcctctctctgcagtttgagtctttcctaatcccctggggtctcattcccactgaggacataggtggcctcctcaggaactctgtgctgggtaacagaatgcgggagtgtgaacctggctctgccacctaccagctgtcactccacctccttgggcctcactctcctcatctgtagaatagggttagcaatagaatccatgtcaccaggttagaatgatgagtcagtggtttgacctccagaaactaatcagcctgatctctgatgccaaataagtattggtgataacgaccacttttatgggaggagcgttcacctgtcaataattcagagatcaacaccttttccttttgtttttcaggattaagaaagaatggtgggatcagattcccaacccagcccgcagccgcctcgtggctataataatccaggatgctcaggtaggagtaggcgtggatgaggacatgtgggactgtgtacatgaagaagtgtggttcagaacacctgggctgttaaggaccttcactggcttctggaatggcaaatagacagtcaggagggttgcaggggagacagagacagaagccgaatgaggtcattagcagaccagaggctttcccgcccttccccttggcaatcccagcctggggtgggcttctctggggttggtttcctgtttttttccctccccttgggagaatgacccttgggtcatcatcactgtgtcattccctggggaggtgccagtaccagggctagaggccagaaggagtggaggaaggagagggtgacaggctttctgtgtcttcttcttaagcataggaaactgcccccgaagcactagcaaatcccttccgggttctcattggcctgaaatgtatcccacccctaagccaggggtggagtcagcttccccaaggcgatggtcctgtgggtgagtgggtggggtttgcctgagcaagatgagagttctctaggtaggagaaagggggattataggtcctgtctagaagagaaggtctgagggtccttgcttttccagggactctggaatctagtggtgttggctttgaatcctgactctgccactcactggcagtgtggacttgagcaagttgcttaattctctgagcctcagtttcctcttgtgggttataacagtgtttacctggtaggacagatattggaatttattgagacaatacatataaagtgcatattccagcctcttgcaaataccaagtgccatttatgtatcagttagtgtttgctgtgtaacaaacgaccccgaaatgtagagggttacaacaactttatttagcttatgcttctgcaggctggcatttggggctgggctcagcagtgagggtggcgggggaggctgggctgggctgggctgggcagatctgaattgagctgacccgtccccgtagcctccctccgtgtctgacagttggctttttttttttttttctttttctgagacggagttttgctcttattgcccaggagtgcaatggcgtgatcttggctcactgcaacctctgcttcctgggttcaagcaattttcttgcctcagcctcccaagtagctgggattacaggcatgtgccaccacgccaggctaattttgtatttttaatagagatggggtttcttcatgtcggtcaggctggtctggaactcctaatatcaggtgatccacccacttcagcctcccaaagtgctgggattacaggcgtgagccactgcacccagcctagttggctgacttttacctgggacagtgcaggtgcctgagccatgtgcctctcactctccagcaggccggcccaggcttgtttacagagtggctcagttttcaagggtgggaagtcccaaggcttcttgaggcctaggcgcagcactggcatgatatcacttccatcacattctatgggcccaagcaagtcccagggccagtgtagattcaagggatgggaggagattcagagcactcctctgtggccacttttgccatcgaccacagtccctgtaaatattaggacaatgtaattaattcccaggaatctgaagctcagaaagcgtaagtgacctgttggacttctgatctgtgtgatgtcgaggcttgtaccccttcctgagcattgccgtactccaggccgggctgcaaggccactctgctctttcattggctgtctctgtattttaggggtcacagtgggagaagcggtcccgaggccaggaaccagccaagtgcccgtatgtatctgaacttaggtcacagcctgcatgcattgggaaggtgatagaattggagaggcaagcccctagctccatgtctgccttctcttccctgcattcggtaattgccctgtgacattagccttcaagggacggcaggaggaggggtgttctggaaacgtggactgctggccaagccccctgagtttcactggtgtgtcaggtacatggtgataccccttgggagtgctgttatagttaacaaccagagcagccgtgcctgttgttaaaatcttgacctaattgtatacttgtcggcaaatagccactatcctgaacactcccctccttttttttaatatacaggatctcactctgtggcccaggctggtgtgcagtggtgcgatcatagctcactgcaccttcaaactcctgagctcaagtgatcctcccatcttagcctcccgagtagctgatactacagatgtgcattaccacgcctggctattttaaaaggtttttgcctgtaattccagctactcaggaggctgaggcatgagaatcacttgaacccgggaggcagaggttgcagtgagcgcagattgtgccactgcactccagcctgggcgacagagtgagactcttgtctcaaaaaaaataataccaaaaaaagtttttgtaaagacaagctctcgctgtgttgccccgccactgtggcctccttagcttcttccctggggcctgctggacctttccatactccagaaactaaagggggtccaggaccctgcttcaaccctaggatcccgcatcttttttttttttttttttttttggacgcagggtcttgctgtgtccctcaggctggagtgcagtgattcactgcagcctcaaactcgtgggctcaagtgattctctagcctcagccttctaagtagctgggactacagtcatacaccaacatgcccagctaattttccttttttttaattcttgtagagatgtttgagacggcttgggctctgttgcccaggctgttctcaaactcctgagctcaagcgatcctccctcctcagcctcctaaagtgctgggattacaggcgtgagccaccgcacccggcttccatatcctttctaattggtcatggcttgggataatggtgttgcttttaattatcatcatccataaagactttttcttactcaacagatctgagcttgtatttggtgcccaggacatgtgctgggttcccgaaatcccaaagacacagaccctaccctcagggatttctcattctagcaacatagactgatcaattactgattataacgttagaaggcatgtctgaagtagacagccatcaggacatggtgatttcaggctgggctttgaagaatgaataggagtttttcaagtgtcgaaactgaaccctgaccaacctttgcttttgcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta